(19) United States
(12) Patent Application Publication
(10) Pub. No.: US 2025/0295587 A1
(43) Pub. Date: Sep. 25, 2025

(54) COMPLEX LIPID NANOPARTICLES ENCAPSULATING POLYPEPTIDES AND USES THEREOF

(71) Applicant: SAIL BIOMEDICINES, INC., Cambridge, MA (US)

(72) Inventors: Emmanouil KARAGIANNIS, Cambridge, MA (US); Jon GRABENSTATTER, Cambridge, MA (US); Lavanya THAPA, Cambridge, MA (US)

(21) Appl. No.: 18/862,799

(22) PCT Filed: May 5, 2023
(86) PCT No.: PCT/US2023/021094

§ 371 Date: Nov. 4, 2024

Related U.S. Application Data
(60) Provisional application No. 63/339,043, filed on May 6, 2022.

(51) Int. Cl. A61K 9/1271; A61K 9/00; A61K 9/19; A61K 38/26; A61K 38/28

(52) U.S. Cl. CPC A61K 9/1271; A61K 9/0031; A61K 9/0043; A61K 9/0053; A61K 9/19; A61K 38/26; A61K 38/28

(57) ABSTRACT

Disclosed herein are complex lipid particles encapsulating one or more exogenous peptides, polypeptides, or proteins, as well as methods of producing a complex lipid formulation comprising an exogenous peptide, polypeptide, or protein. Also, disclosed herein are modified plant messenger packs (PMPs) formulation encapsulating one or more exogenous peptides, polypeptides, or proteins, and methods of producing a modified PMP formulation comprising an exogenous peptide, polypeptide, or protein.

### CROSS-REFERENCE TO RELATED APPLICATIONS

This application claims benefit of priority to U.S. Provisional Application No. 63/339,043, filed May 6, 2022, which is herein incorporated by reference in its entirety.

### BACKGROUND

Polypeptides (e.g., proteins or peptides) are used in therapies (e.g., for the treatment of a disease or condition), for diagnostic purposes, and as pathogen control agents. However, current methods of delivering polypeptides to cells may be limited by the mechanism of delivery, e.g., the efficiency of delivery of the polypeptide to a cell. Therefore, there is a need in the art for methods and compositions for the delivery of polypeptides to cells.

### SUMMARY OF THE INVENTION

One aspect of the invention relates to a method for delivering a therapeutic peptide or protein to a human subject in need thereof. The method comprises orally or enterally administering to the human subject a pharmaceutical preparation comprising:
(a) a plurality of complex lipid particles characterized by: (i) comprising at least 10 plant lipids extracted from one or more plant sources; (ii) comprising a sterol exogenous to the one or more plant sources, (iii) comprising a polyethylene glycol (PEG)-conjugated lipid; (iii) containing less than 10% w/w of protein matter endogenous to the one or more plant sources; and (iv) containing less than 10 mol % of exogenous ionizable lipids; and
(b) the therapeutic peptide or protein encapsulated in the complex lipid particles.

In some embodiments, the therapeutic peptide or protein is a hormone or glucagon-like peptide 1 (GLP-1) agonist. In one embodiment, the therapeutic peptide or protein is insulin, exenatide, semaglutide, or tirzepatide.

In some embodiments, the therapeutic peptide or protein is delivered to a brain tissue in the human subject.

In some embodiments, the complex lipid particle contains ten or more lipids belonging to one or more of the sub-classes selected from the group consisting of acylsterylglycosides, ceramides, digalactosyldiacylglycerols, diacylglyceryl glucuronides, hemibismonoacylglycerophosphates, hexosylceramides, lysophosphatidylcholines, lysophosphatidylethanolamines, monogalactosyldiacylglycerols, phosphatidylcholines, phosphatidylethanolamines, phosphatidylethanols, phosphatidylglycerols, phosphatidylinositols, sulfoquinovosyl diacylglycerols, and sterols.

In some embodiments, the complex lipid particle contains lipids from at least five, at least six, at least seven, at least eight, at least nine, or at least ten different sub-classes.

In some embodiments, the complex lipid particle contains less than 5% w/w of protein matter endogenous to the one or more plant sources.

In some embodiments, the complex lipid particle contains less than 5 mol % of exogenous ionizable lipids.

In some embodiments, at least one of the plant sources is grapefruit, lemon, dragon fruit, spinach, kale, strawberry, broccoli, or soy.

In some embodiments, the complex lipid particle comprises:
about 85-95% w/w of the plant lipids,
about 5-8% w/w of the sterol,
about 1-3.5% w/w the polyethylene glycol (PEG)-lipid conjugate, based on the amounts of total lipids in the complex lipid formulation.

Another aspect of the invention relates to a complex lipid formulation, comprising a plurality of complex lipid particles, each complex lipid particle of the plurality comprising at least five lipids extracted from one or more plant sources and at least two exogenous lipids; and one or more exogenous peptides, polypeptides, or proteins, encapsulated in the complex lipid particles. The complex lipid particles are characterized by one or more of the following characteristics:
i) containing less than 50% w/w of protein matter endogenous to the one or more plant sources; and
ii) containing less than 50 mol % of ionizable lipids.

In some embodiments, the complex lipid particle contains 5-1000 lipids extracted from one or more plant sources. In some embodiments, the complex lipid particle contains at least 10 plant lipids belonging to one or more of the classes selected from the group consisting of glycerolipid, sphingolipid, and sterol. For instance, the complex lipid particle contains at least 15, at least 20, at least 25, at least 30, at least 35, at least 40, at least 45, at least 50, at least 60, at least 70, at least 80, at least 90, at least 100, at least 150, at least 200, at least 250, at least 300, at least 400, at least 500, at least 600, at least 700, or at least 800 plant lipids belonging to one or more of the classes selected from the group consisting of glycerolipid, sphingolipid, and sterol. In some embodiments, the complex lipid particle contains lipids from at least two or at least three of these different classes.

In some embodiments, the complex lipid particle contains one or more glycerolipids selected from the group consisting of phospholipids (PL), galactolipids (GL), triacylglycerols (TG), and sulfolipids (SL). In some embodiments, the complex lipid particle contains one or more sphingolipids selected from the group consisting of glycosyl inositolphosphoceramides (GIPC), glucosylceramides (GCer), ceramides (Cer), and free long-chain bases (LCB). In some embodiments, the complex lipid particle contains one or more phytosterols selected from the group consisting of campesterol, stigmasterol, and sitosterol.

In some embodiments, the complex lipid particle contains one or more lipids belonging to one or more of the sub-classes selected from the group consisting of acyl diacylglyceryl glucuronides, acylhexosylceramides, acylsterylglycosides, bile acids, acyl carnitines, cholesteryl esters, ceramides, cardiolipins, coenzyme Qs, diacylglycerols, digalactosyldiacylglycerols, diacylglyceryl glucuronides, dilysocardiolipins, fatty acids, fatty acid esters of hydroxyl fatty acids, hemibismonoacylglycerophosphates, hexosylceramides, lysophosphatidic acids, lysophosphatidylcholines, lysophosphatidylethanolamines, N-acyl-lysophosphatidylethanolamines, lysophosphatidylglycerols, lysophosphatidylinositols, lysophosphatidylserines, monogalactosyldiacylglycerols, lysocardiolipins, N-acyl ethanolamines, N-acyl glycines, N-acyl glycyl serines, phosphatidic acids, phosphatidylcholines, phosphatidylethanolamines, phosphatidylethanols, phosphatidylglycerols, phosphatidylinositols, ceramide phosphoinositols, phosphatidylmethanols, phosphatidylserines, steryl esters, stigmasterols, sulfatides, sulfonolipids, sphingomyelins, sulfoquinovosyl diacylglycerols, sterols, and triacylglycerols. In some embodiments, the complex lipid particle contains at least 10 plant lipids belonging to one or more of the sub-classes selected from the group consisting of the sub-classes listed above. For instance, the complex lipid particle contains at least 15, at least 20, at least 25, at least 30, at least 35, at least 40, at least 45, at least 50, at least 60, at least 70, at least 80, at least 90, at least 100, at least 150, at least 200, at least 250, at least 300, at least 400, at least 500, at least 600, at least 700, or at least 800 plant lipids belonging to one or more of the sub-classes selected from the group consisting of the sub-classes listed above.

In some embodiments, the complex lipid particle contains 10 or more lipids belonging to one or more of the sub-classes selected from the group consisting of acylsterylglycosides, ceramides, digalactosyldiacylglycerols, diacylglyceryl glucuronides, hemibismonoacylglycerophosphates, hexosylceramides, lysophosphatidylcholines, lysophosphatidylethanolamines, monogalactosyldiacylglycerols, phosphatidylcholines, phosphatidylethanolamines, phosphatidylethanols, phosphatidylglycerols, phosphatidylinositols, sulfoquinovosyl diacylglycerols, and sterols. For instance, the complex lipid particle contains at least 15, at least 20, at least 25, at least 30, at least 35, at least 40, at least 45, at least 50, at least 60, at least 70, at least 80, at least 90, at least 100, at least 150, at least 200, at least 250, at least 300, at least 400, at least 500, at least 600, at least 700, or at least 800 plant lipids belonging to one or more of the sub-classes selected from the group consisting of the sub-classes listed above.

In some embodiments, the complex lipid particle contains lipids from at least five, at least six, at least seven, at least eight, at least nine, or at least ten different sub-classes listed above.

The complex lipid particle contains less than 50% w/w of protein matter endogenous to the one or more plant sources. For instance, the complex lipid particle contains less than 45% w/w, less than 40% w/w, less than 35% w/w, less than 30% w/w, less than 25% w/w, less than 20% w/w, less than 15% w/w, less than 10% w/w, less than 9% w/w, less than 8% w/w, less than 7% w/w, less than 6% w/w, less than 5% w/w, less than 4% w/w, less than 3% w/w, less than 2% w/w, less than 1% w/w, less than 0.5% w/w, less than 0.1% w/w, or essentially free of protein matter endogenous to the one or more plant sources. In some embodiments, the complex lipid particle contains less than 30% w/w of protein matter endogenous to the one or more plant sources. In some embodiments, the complex lipid particle contains less than 20% w/w of protein matter endogenous to the one or more plant sources. In some embodiments, the complex lipid particle contains less than 10% w/w of protein matter endogenous to the one or more plant sources. In some embodiments, the complex lipid particle contains less than 5% w/w of protein matter endogenous to the one or more plant sources.

The complex lipid particle may comprise reduced or minimized residual dsDNA matter endogenous to the one or more plant sources. For instance, the complex lipid particle may contain less than 15% w/w, less than 10% w/w, less than 5% w/w, less than 1% w/w, less than 0.5% w/w, less than 0.1% w/w, less than 0.05% w/w, less than 0.01% w/w, less than 0.005% w/w, less than 0.001% w/w, or essentially free of residual dsDNA matter endogenous to the one or more plant sources. In some instances, the lipid bilayer of the complex lipid particle does not contain residual dsDNA. In some embodiments, the complex lipid particle contains less than 1% w/w of residual dsDNA matter endogenous to the one or more plant sources. In some embodiments, the complex lipid particle contains less than 0.1% w/w of residual dsDNA matter endogenous to the one or more plant sources. In some embodiments, the complex lipid particle contains less than 0.01% w/w of residual dsDNA matter endogenous to the one or more plant sources.

The complex lipid particle contains less than 50 mol % of ionizable lipids (e.g., ionizable lipids exogenous to the one or more plant sources). For instance, the complex lipid particle contains less than 45 mol %, less than 40 mol %, less than 35 mol %, less than 30 mol %, less than 25 mol %, less than 20 mol %, less than 15 mol %, less than 10 mol %, less than 9 mol %, less than 8 mol %, less than 7 mol %, less than 6 mol %, less than 5 mol %, less than 4 mol %, less than 3 mol %, less than 2 mol %, less than 1 mol %, less than 0.5 mol %, less than 0.1 mol %, or essentially free of ionizable lipids (e.g., ionizable lipids exogenous to the one or more plant sources). In some embodiments, the complex lipid particle contains less than 20 mol % of exogenous ionizable lipids. In some embodiments, the complex lipid particle contains less than 5 mol % of exogenous ionizable lipids. In some embodiments, the complex lipid particle is essentially free of exogenous ionizable lipids.

In some embodiments, the complex lipid formulation does not contain an exogenous nucleic acid.

In some embodiments, at least one of the plant sources is a citrus fruit. For instance, the citrus fruit may be a grapefruit or a lemon.

In some embodiments, at least one of the plant sources is a non-citrus plant. For instance, the non-citrus plant may be a dragon fruit, spinach, kale, strawberry, broccoli, or soy.

In some embodiments, the one or more plant sources may be a citrus fruit, a non-citrus plant, or a combination thereof. In some embodiments, the one or more plant sources may be a grapefruit, lemon, dragon fruit, spinach, kale, strawberry, broccoli, soy, or combination thereof.

In some embodiments, the exogenous lipids comprise a sterol and a polyethylene glycol (PEG)-lipid conjugate.

In some embodiments, the exogenous lipids further comprise a lipid selected from the group consisting of a fatty acid, a glycerolipid, a glycerophospholipid, a sphingolipid, a second sterol, and an additive synthetic lipid. In some embodiments, the exogenous lipids further comprise phosphatidylglycerol (PS), phosphatidylcholine (PC), phosphatidylglycerol (PG), phosphatidylethanolamine (PE), soy PS, soy PC, soy PG, soy PE, arachidonic acid, glucosyl sitosterol, glucosylceramide, MGDG, DOPC, DLPC, DLPE, DGTS, DGDG, or a mixture thereof.

In some embodiments, the complex lipid particle comprises about 10-95% w/w of the plant lipids. For instance, the complex lipid particle comprises about 25-95% w/w, about 30-95% w/w, about 35-95% w/w, about 40-95% w/w, about 45-95% w/w, about 50-95% w/w, about 55-95% w/w, about 60-95% w/w, about 65-95% w/w, about 70-95% w/w, about 75-95% w/w, about 80-95% w/w, or about 85-95% w/w of the plant lipids based on the amounts of total lipids in the complex lipid formulation.

In some embodiments, the complex lipid particle comprises:
about 10-95% w/w of the plant lipids,
about 5-60% w/w of the sterol,
about 0.5-15% w/w the polyethylene glycol (PEG)-lipid conjugate, based on the amounts of total lipids in the complex lipid formulation.

In some embodiments, the complex lipid particle comprises:
about 85-95% w/w of the plant lipids,
about 5-8% w/w of the sterol,
about 1-3.5% w/w the polyethylene glycol (PEG)-lipid conjugate, based on the amounts of total lipids in the complex lipid formulation.

Another aspect of the invention relates to a modified plant messenger pack (PMP) formulation comprising one or more PMPs modified with one or more sterols and one or more polyethylene glycol (PEG)-lipid conjugates, wherein the modified PMPs are formulated with one or more exogenous peptides, polypeptides, or proteins, and wherein the one or more exogenous peptides, polypeptides, or proteins are encapsulated by the modified PMP.

In some embodiments, the PMP comprises a purified plant extracellular vesicle (EV), or a segment or extract thereof. In some embodiments, the EV or segment or extract thereof is obtained from a citrus fruit, e.g., a grapefruit or a lemon.

In some embodiments, the PMP is obtained from a citrus fruit, e.g., a grapefruit or a lemon.

In some embodiments, the modified PMP is a lipophilic moiety selected from the group consisting of a lipoplex, a liposome, a lipid nanoparticle, a polymer-based carrier, an exosome, a lamellar body, a micelle, and an emulsion. In one embodiment, the modified PMP is a liposome selected from the group consisting of a cationic liposome, a nanoliposome, a proteoliposome, a unilamellar liposome, a multilamellar liposome, a ceramide-containing nanoliposome, and a multivesicular liposome. In one embodiment, the modified PMP is a lipid nanoparticle.

In any of the above aspects of the invention relating to a complex lipid formulation or a modified PMP formulation, the following embodiments would be applicable.

The exogenous peptides, polypeptides, or proteins may be therapeutic agents.

In some embodiments, the exogenous peptide, polypeptide, or protein is an enzyme. In some embodiments, the enzyme is a recombination enzyme or an editing enzyme.

In some embodiments, the exogenous peptide, polypeptide, or protein is an antibody or an antibody fragment.

In some embodiments, the exogenous peptide, polypeptide, or protein is an Fc fusion protein.

In some embodiments, the exogenous peptide, polypeptide, or protein is a hormone. In some embodiments, the exogenous peptide, polypeptide, or protein is insulin.

In some embodiments, the exogenous peptide, polypeptide, or protein is a peptide.

In some embodiments, the exogenous peptide, polypeptide, or protein is a receptor agonist or a receptor antagonist. In some embodiments, the exogenous peptide, polypeptide, or protein is a glucagon-like peptide 1 (GLP-1) agonist. In some embodiments, the exogenous peptide, polypeptide, or protein is exenatide, semaglutide, or tirzepatide.

In some embodiments, the exogenous peptide, polypeptide, or protein is an antibody of Table 1, a peptide of Table 2, an enzyme of Table 3, or a protein of Table 4.

In some embodiments, the exogenous peptide, polypeptide, or protein has a size of less than 100 kD, less than 90 kD, less than 80 kD, less than 70 kD, less than 60 kD, less than 50 kD, less than 40 kD, less than 30 kD, less than 20 kD, or less than 10 kD. In some embodiments, the exogenous peptide, polypeptide, or protein has a size of less than 50 kD. In some embodiments, the exogenous peptide, polypeptide, or protein is at least 3 kD, at least 4 kD, or at least 5 kD in size. In some embodiments, the exogenous peptide, polypeptide, or protein has a size of at least 3 kD. In some embodiments, the exogenous peptide, polypeptide, or protein is at least 5 kD in size.

In some embodiments, the exogenous peptide, polypeptide, or protein comprises at least 10, at least 20, at least 30, at least 40 or at least 50 amino acid residues. In some embodiments, the exogenous peptide, polypeptide, or protein comprises at least 30 amino acid residues. In some embodiments, the exogenous peptide, polypeptide, or protein comprises at least 50 amino acid residues.

In some embodiments, the exogenous peptide, polypeptide, or protein has an overall charge that is neutral. In some embodiments, the exogenous peptide, polypeptide, or protein has been modified to have a charge that is neutral. In some embodiments, the exogenous peptide, polypeptide, or protein has an overall charge that is positive. In some embodiments, the exogenous peptide, polypeptide, or protein has an overall charge that is negative.

In some embodiments, the exogenous peptides, polypeptides, or proteins may be modified (e.g. lipid modified such as a lipid tail). In some embodiments, the exogenous peptides, polypeptides, or proteins may be a lipopeptide. In some embodiments, the exogenous peptides, polypeptides, or proteins may be synthetic or contain synthetic amino acids.

In some embodiments, the sterol is cholesterol or sitosterol.

In some embodiments, the PEG-lipid conjugate is a C14-PEG2k or C18-PEG2k. In some embodiments, the PEG-lipid conjugate is a PEG-DMG or PEG-PE. In some embodiments, the PEG-DMG is PEG2000-DMG or PEG2000-PE. In some embodiments, the PEG-lipid conjugate is a PEG2000-PE, PEG2000-DMG, PEG2000-DSPE, or a derivative thereof. In some embodiments, the PEG-lipid conjugate is a C18-PEG2000 PE or its derivative. For instance, the PEG-lipid conjugate is DSPE-PEG2000.

In some embodiments, the sterol is cholesterol or sitosterol, and the PEG-lipid conjugate is a C18-PEG2000 PE or its derivative. In some embodiments, the sterol is cholesterol or sitosterol, and the PEG-lipid conjugate is DSPE-PEG2000.

In some embodiments, the concentration of the sterol in the complex lipid particle or in the modified PMP ranges from about 5 to 60% w/w, for instance, from about 5 to 50% w/w, from about 5 to 40% w/w, from about 5 to 30% w/w, from about 5 to 20% w/w, from about 5 to 15% w/w, from about 0.5 to 15% w/w, from about 5 to 8% w/w, or from about 6 to 7% w/w, based on the amounts of total lipids in the complex lipid particle or in the modified PMP. In some embodiments, the sterol ranges from about 15 to 20% w/w, from about 20 to 30% w/w, from about 30 to 40% w/w, from about 40 to 50% w/w, or from about 50 to 60% w/w, based on the amounts of total lipids in the complex lipid particle or in the modified PMP.

In some embodiments, the concentration of the PEG-lipid conjugate ranges from about 0.5 to 5% w/w, from about 0.5 to 3.5% w/w, from about 1 to 3.5% w/w, from about 0.5 to 3% w/w, from about 1 to 3% w/w, from about 0.5 to 2.5% w/w, from about 1 to 2.5% w/w, from about 1.5 to 2.5% w/w, or from about 2 to 2.5% w/w, based on the amounts of total lipids in the complex lipid particle or in the modified PMP. In some embodiments, the concentration of the PEG-lipid conjugate ranges from about 0.5 to 15% w/w, from about 1 to 15% w/w, from about 2 to 5% w/w, from about 5 to 8% w/w, from about 8 to 12% w/w, or from about 12 to 15% w/w, based on the amounts of total lipids in the complex lipid particle or in the modified PMP.

In some embodiments, the sterol is cholesterol or sitosterol having a concentration ranging from about 5 to 50% w/w, based on the amounts of total lipids in the complex lipid particle or in the modified PMP; and the PEG-lipid conjugate is a PEG2000-PE, PEG2000-DMG, PEG2000-DSPE, or a derivative thereof having a concentration ranging from about 1 to 3.5% w/w, based on the amounts of total lipids in the complex lipid particle or in the modified PMP.

In some embodiments, the sterol is cholesterol or sitosterol having a concentration ranging from about 5 to 8% w/w, based on the amounts of total lipids in the complex lipid particle or in the modified PMP; and the PEG-lipid conjugate is a C18-PEG2000 PE or its derivative having a concentration ranging from about 1 to 3.5% w/w, based on the amounts of total lipids in the complex lipid particle or in the modified PMP.

In some embodiments, the sterol is cholesterol or sitosterol having a concentration ranging from about 20 to 25% w/w, based on the amounts of total lipids in the complex lipid particle or in the modified PMP; and the PEG-lipid conjugate is a DSPE-PEG2000 or its derivative having a concentration ranging from about 1 to 3.5% w/w, based on the amounts of total lipids in the complex lipid particle or in the modified PMP.

In some embodiments, the sterol is cholesterol or sitosterol having a concentration ranging from about 5 to 8% w/w, based on the amounts of total lipids in the complex lipid particle or in the modified PMP; and the PEG-lipid conjugate is a C18-PEG2000 PE or its derivative having a concentration ranging from about 1 to 3.5% w/w, based on the amounts of total lipids in the complex lipid particle or in the modified PMP.

The complex lipid particle or modified PMP may have an average size of less than about 400 nm, less than about 350 nm, less than about 300 nm, less than about 250 nm, or less than about 200 nm. In one embodiment, the complex lipid particle or modified PMP has an average size of less than about 200 nm. In one embodiment, the complex lipid particle or the modified PMP has an average size of about 100 to 180 nm. In one embodiment, the complex lipid particle or the modified PMP has an average size of about 100 to 160 nm.

The complex lipid particle or modified PMP may have a polydispersity index (PDI) of less than about 0.7, less than about 0.6, less than about 0.5, or less than about 0.4. For instance, the complex lipid particle or modified PMP may have a PDI ranging from about 0.1 to about 0.7, from about 0.1 to about 0.6, from about 0.1 to about 0.5, from about 0.1 to about 0.4, from about 0.2 to about 0.7, from about 0.2 to about 0.6, from about 0.2 to about 0.5, or from about 0.2 to about 0.4. In one embodiment, the complex lipid particle has a PDI of about 0.1 to about 0.5. In one embodiment, the complex lipid particle has a PDI of about 0.2 to about 0.4.

In some embodiments, the complex lipid particle formulation or the modified PMP formulation, e.g., the aqueous phase, further comprises phosphate, citrate, sodium bicarbonate, HEPES, TAE, or TRIS buffer. In some embodiments, the complex lipid particle formulation or the modified PMP formulation further comprises water, PBS, or bicarbonate. In some embodiments, the complex lipid particle formulation or the modified PMP formulation further comprises a bicarbonate buffer having a molarity of 0.001M to 0.1M. The buffer solution may have a pH of about 3.0 to about 8.5. The HEPES or TRIS buffer may have a pH of about 7.0 to about 8.5. The HEPES or TRIS buffer can be at a concentration of about 7 mg/mL to about 15 mg/mL. The aqueous phase may further comprise about 2.0 mg/mL to about 4.0 mg/mL of NaCl. In one embodiment, the aqueous phase comprises a citrate buffer having a pH of about 3.0 to about 3.2. In one embodiment, the aqueous phase comprises a sodium bicarbonate buffer having a pH of about 8.0 to about 8.2.

In some embodiments, the complex lipid particle formulation or modified PMP formulation further comprises one or more cryoprotectants. The one or more cryoprotectants may be sucrose, glycerol, mannitol, or a combination thereof. In one embodiment, the modified PMP formulation comprises 2-5% sucrose, 2-5% mannitol, or a combination thereof. In one embodiment, the modified PMP formulation comprises 0-0.5% sucrose, 0-0.5% mannitol, or a combination thereof. In one embodiment, the modified PMP comprises 0.5-2% sucrose, 0.5-2% mannitol, or a combination thereof.

In some embodiments, the complex lipid particle formulation or modified PMP formulation is a lyophilized composition. The lyophilized composition may comprise one or more lyoprotectants. The lyophilized composition may comprise a poloxamer, potassium sorbate, sucrose, or any combination thereof. In one embodiment, the lyophilized composition comprises a poloxamer, e.g., poloxamer 188.

In some embodiments, the complex lipid particle formulation is not lyophilized. In some embodiments, the complex lipid formulation is a liquid composition.

In some embodiments, the complex lipid particle formulation or modified PMP formulation is stable for at least one day at room temperature, and/or stable for at least one day, at least one week, or at least one month at 4° C., with or without lyophilization. In some embodiments, the complex lipid particle formulation or modified PMP formulation is stable for at least 24 hours, 48 hours, seven days, or 30 days at 4° C., with or without lyophilization. In one embodiment, the complex lipid formulation is stable at room temperature, and/or at 4° C. for at least two weeks, without lyophilization. In some embodiments, the complex lipid particle formulation or modified PMP formulation is stable at a temperature of at least 20° C., 24° C., or 37° C.

Some embodiments provide a composition comprising a plurality of the modified PMP formulations of any of the above embodiments. In some embodiments, the modified PMP formulations in the composition are at a concentration effective to increase the fitness of a mammal.

Some embodiments provide a composition comprising a plurality of the complex lipid particle formulations of any of the above embodiments. In some embodiments, the complex lipid particle formulations in the composition are at a concentration effective to increase the fitness of a mammal.

In some embodiments, the exogenous peptide, polypeptide, or protein in the complex lipid particle formulation is at a concentration of at least 0.01, 0.1, 0.2, 0.3, 0.4, 0.5, or 1 μg/mL. In some embodiments, the exogenous peptide, polypeptide, or protein is at a concentration of at least 2, 5, 10, 20, 30, 40, 50, 60, 70, 80, 90, 100, 150, or 200 μg/mL.

In some embodiments, at least 15% of the complex lipid particles in the plurality of the complex lipid particle formulations encapsulate the exogenous peptide, polypeptide, or protein. In some embodiments, at least 50% of the complex lipid particles in the plurality of the complex lipid particle formulations encapsulate the exogenous peptide, polypeptide, or protein. In some embodiments, at least 95% of the complex lipid particles in the plurality of the complex lipid particle formulations encapsulate the exogenous peptide, polypeptide, or protein.

In some embodiments, at least 15% of the modified PMPs in the plurality of the modified PMP formulations encapsulate the exogenous peptide, polypeptide, or protein. In some embodiments, at least 50% of the modified PMPs in the plurality of the modified PMP formulations encapsulate the exogenous peptide, polypeptide, or protein. In some embodiments, at least 95% of the modified PMPs in the plurality of the modified PMP formulations encapsulate the exogenous peptide, polypeptide, or protein.

Another aspect of the invention relates to a pharmaceutical composition or a pharmaceutical preparation comprising the complex lipid formulation as described herein, and a pharmaceutically acceptable vehicle, carrier, or excipient.

In some embodiments, the pharmaceutical composition or pharmaceutical preparation is in an oral dosage form, such as a capsule dosage form or a tablet dosage form.

All above descriptions and all embodiments discussed in the above aspect relating to the complex lipid formulation or the complex lipid particles are applicable to these aspects of the invention relating to a pharmaceutical formulation comprising the complex lipid formulation.

In another aspect, the disclosure features a pharmaceutical composition comprising the modified PMP formulation according to any one of the above embodiments and a pharmaceutically acceptable vehicle, carrier, or excipient.

In some embodiments, the pharmaceutical composition or pharmaceutical preparation is formulated for administration to a mammal such as a human subject. In some embodiments, the pharmaceutical composition or pharmaceutical preparation is formulated for administration to a mammalian cell.

In any of the above aspects of the invention relating to a pharmaceutical composition or pharmaceutical preparation, the following embodiments would be applicable.

Another aspect of the invention relates to a method of producing a complex lipid formulation comprising a plurality of complex lipid particles encapsulating an exogenous peptide, polypeptide, or protein. The method comprises:
extracting at least five lipids from one or more plant sources;
mixing at least two exogenous lipids with the extracted plant lipids to form complex lipid particles; and
loading the complex lipid particles with the exogenous peptide, polypeptide, or protein, wherein the loading causes the exogenous peptide, polypeptide, or protein to be encapsulated by the complex lipid particles, thereby forming the complex lipid formulation.

All above descriptions and all embodiments discussed in the above aspect relating to the complex lipid formulation or the complex lipid particles are applicable to these aspects of the invention relating to a method of producing a complex lipid formulation.

In some embodiments, the lipids are extracted from one or more plant sources by adding to the plant sources an extraction solvent comprising methanol, ethanol, propanol, 1-buthanol, acetonitrile, acetone, dimethylformamide, tetrahydrofuran, dimethyl sulfoxide, methyl tert-butyl ether, chloroform, ethyl acetate, or a mixture thereof. In some embodiments, the extraction solvent is dichloromethane:methanol, chloroform:methanol, methanol:methyl tert-butyl ether (MTBE), dimethylformamide:methanol; acetonitrile:methanol; acetone:methanol; tetrahydrofuran:methanol; dimethyl sulfoxide:methanol; acetonitrile:ethanol; or ethyl acetate:ethanol.

In some embodiments, the extracting step further comprises reducing the protein matter endogenous to the one or more plant sources to less than 50% w/w, less than 45% w/w, less than 40% w/w, less than 35% w/w, less than 30% w/w, less than 25% w/w, less than 20% w/w, less than 15% w/w, less than 10% w/w, less than 9% w/w, less than 8% w/w, less than 7% w/w, less than 6% w/w, less than 5% w/w, less than 4% w/w, less than 3% w/w, less than 2% w/w, less than 1% w/w, less than 0.5% w/w, or less than 0.1% w/w, or essentially completely eliminating the protein matter endogenous to the one or more plant sources.

The complex lipid particle may comprise reduced or minimized residual dsDNA matter endogenous to the one or more plant sources. For instance, the complex lipid particle may contain less than 15% w/w, less than 10% w/w, less than 5% w/w, less than 1% w/w, less than 0.5% w/w, less than 0.1% w/w, less than 0.05% w/w, less than 0.01% w/w, less than 0.005% w/w, less than 0.001% w/w, or essentially free of residual dsDNA matter endogenous to the one or more plant sources. In some instances, the lipid bilayer of the complex lipid particle does not contain residual dsDNA. In some embodiments, the complex lipid particle contains less than 1% w/w of residual dsDNA matter endogenous to the one or more plant sources. In some embodiments, the complex lipid particle contains less than 0.1% w/w of residual dsDNA matter endogenous to the one or more plant sources. In some embodiments, the complex lipid particle contains less than 0.01% w/w of residual dsDNA matter endogenous to the one or more plant sources.

In some embodiments, the mixing step is carried out by thin film mixing or microfluidics mixing.

In some embodiments, the exogenous lipids comprise a sterol and a polyethylene glycol (PEG)-lipid conjugate.

In some embodiments, the exogenous lipids do not include an ionizable lipid. Thus, the complex lipid formulation is prepared without adding exogenous ionizable lipid.

In another aspect, the disclosure features a method of producing a modified PMP formulation comprising an exogenous peptide, polypeptide, or protein, the method comprising: (a) providing a solution comprising a modified PMP containing one or more PMPs, one or more sterols, and one or more polyethylene glycol (PEG)-lipid conjugates; providing a solution comprising the exogenous peptide, polypeptide, or protein; and (b) loading the modified PMP with the exogenous peptide, polypeptide, or protein, wherein the loading causes the exogenous peptide, polypeptide, or protein to be encapsulated by the modified PMP.

In some embodiments, the exogenous peptide, polypeptide, or protein is soluble in the solution.

In some embodiments, the loading comprises one or more of sonication, electroporation, and lipid extrusion. In some embodiments, the loading comprises sonication and lipid extrusion. In some embodiments, the loading comprises lipid extrusion. In some embodiments, the PMP lipids are isolated prior to lipid extrusion. In some embodiments, the isolated PMP lipids comprise glycosylinositol phosphorylceramides (GIPCs).

Another aspect of the invention relates to a method for delivering a peptide, polypeptide, or protein to a mammalian cell or a mammal. The method comprises contacting the mammalian cell with or administering to the mammal a complex lipid formulation, under conditions sufficient to allow uptake of the complex lipid formulation by the mammalian cell or by the mammal. The complex lipid formulation comprises:
a plurality of complex lipid particles, each complex lipid particle of the plurality comprising at least five plant lipids and at least two exogenous lipids; and
one or more exogenous peptides, polypeptides, or proteins, encapsulated in the complex lipid particles, wherein the complex lipid particles are characterized by one or more of the following characteristics:
i) containing less than 50% w/w of protein matter endogenous to the one or more plant sources; and
ii) containing less than 50 mol % of ionizable lipids.

In some embodiments, the mammalian cell is a cell in a human, or the mammal is a human. In some embodiments, the uptake by the mammalian cell or by the mammal of the exogenous peptide, polypeptide, or protein encapsulated by the complex lipid particles is increased relative to the uptake of the exogenous peptide, polypeptide, or protein not encapsulated by a complex lipid particle.

In some embodiments, the method is for delivering a peptide, polypeptide, or protein to a mammal, and the administration is via oral, enteral, intranasal, intrarectal (including intracolonic), or intrajejunal route.

In some embodiments, the mammalian cell is a brain cell.

Another aspect of the invention relates to a method for treating or preventing a disease or disorder in a subject for which a therapeutic agent is indicated. The method comprises administering to the subject in need thereof an effective amount of a complex lipid formulation comprising:
a plurality of complex lipid particles, each complex lipid particle of the plurality comprising at least five lipids extracted from one or more plant sources and at least two exogenous lipids; and
one or more exogenous peptides, polypeptides, or proteins, encapsulated in the complex lipid particles, wherein the complex lipid particles are characterized by one or more of the following characteristics:
i) containing less than 50% w/w of protein matter endogenous to the one or more plant sources; and
ii) containing less than 50 mol % of ionizable lipids.

In some embodiments, the administration is via oral, enteral, intranasal, intrarectal (including intracolonic), or intrajejunal route.

In some embodiments, the disease is diabetes, and the exogenous peptide, polypeptide, or protein is insulin, exenatide, semaglutide, or tirzepatide.

In another aspect, the disclosure features a method for delivering a peptide, polypeptide, or protein to a mammalian cell, the method comprising contacting the cell with the modified PMP formulation according to any of the above embodiments, wherein the contacting is performed with an amount and for a time sufficient to allow uptake of the modified PMP formulation by the cell. In some embodiments, the cell is a cell in a subject.

In some embodiments, the exogenous peptide, polypeptide, or protein is released from the modified PMP formulation in the mammalian cell with which the modified PMP formulation is contacted. In some embodiments, the exogenous peptide, polypeptide, or protein exerts activity in the cytoplasm of the mammalian cell. In some embodiments, the exogenous peptide, polypeptide, or protein is translocated to the nucleus of the mammalian cell. In some embodiments, the exogenous peptide, polypeptide, or protein exerts activity in the nucleus of the mammalian cell.

In another aspect, the disclosure features a method for delivering a peptide, polypeptide, or protein to a mammal, the method comprising administering to the mammal the modified PMP formulation according to any of the above embodiments, wherein the administering is performed under conditions sufficient to allow uptake of the modified PMP formulation by the mammal.

In another aspect, the disclosure features a modified PMP formulation, composition, pharmaceutical composition, or method of any of the above embodiments, wherein the mammal is a human.

In another aspect, the disclosure features a modified PMP formulation, composition, pharmaceutical composition, or method of any of the above embodiments, wherein the uptake by a cell of the exogenous peptide, polypeptide, or protein encapsulated by the modified PMP formulation is increased relative to uptake of the exogenous peptide, polypeptide, or protein not encapsulated by a modified PMP formulation.

In another aspect, the disclosure features a modified PMP formulation, composition, pharmaceutical composition, or method of any of the above embodiments, wherein the effectiveness of the exogenous peptide, polypeptide, or protein encapsulated by the modified PMP formulation is increased relative to the effectiveness of the exogenous peptide, polypeptide, or protein not encapsulated by a modified PMP formulation.

In another aspect, the disclosure features a method for treating or preventing a disease or disorder in a subject for which a therapeutic agent is indicated, the method comprising administering to the subject in need thereof an effective amount of the modified PMP formulation according to any of the above embodiments, wherein the therapeutic agent is the exogenous peptide, polypeptide, or protein encapsulated by the modified PMP in the modified PMP formulation.

In some embodiments, the disease or disorder is diabetes. In some embodiments, the administration of the modified PMP formulations lowers the blood sugar of the subject. In some embodiments, the exogenous peptide, polypeptide, or protein is insulin.

In another aspect, the disclosure features a modified PMP formulation, composition, pharmaceutical composition, or method of any of the above embodiments, wherein the modified PMP formulation is not significantly degraded by gastric fluids, e.g., is not significantly degraded by fasted gastric fluids.

In another aspect, the disclosure features a method of any of the above embodiments, wherein the administration is via oral, enteral, intranasal, or intrarectal (including intracolonic) route.

### BRIEF DESCRIPTION OF THE DRAWINGS

FIG. 1 is a scheme showing an exemplary formulation process for preparing the complex lipid particles containing the natural source lipids and exogenous lipids, as described in Example 2.

FIG. 2 is a scheme showing an exemplary process for loading a bioactive molecule in to complex lipid particles containing the natural source lipids and exogenous lipids, as described in Example 2.

FIG. 3 is a graph showing the insulin concentration in the plasma of the mice at one hour after intrarectal administration of the insulin-loaded complex lipid formulation containing the natural source lipids and exogenous lipids. (†) The dashed line reflects the benchmark for fusogenic liposomes containing insulin administered directly into colon (10 U/kg, 12 minutes post injection).

FIG. 4 is a graph showing the insulin concentration in the brain of the mice at two hours after intranasal administration of the insulin-loaded complex lipid formulation containing the natural source lipids and exogenous lipids. The dashed lines reflect the benchmark for brain delivery of 10 U/kg insulin boosted by intranasal co-administration with (†) cell-penetrating peptides and (‡) free insulin 90 minutes post administration.

### DETAILED DESCRIPTION OF THE INVENTION

### I. Definitions

As used herein, the term “encapsulate” or “encapsulated” refers to an enclosure of a moiety (e.g., an exogenous peptide, polypeptide, or protein as defined herein) within an enclosed lipid membrane structure, e.g., a lipid bilayer. The lipid membrane structure may be, e.g., a plant messenger pack (PMP) or a plant extracellular vesicle (EV), or may be obtained from or derived from a plant EV. An encapsulated moiety (e.g., an encapsulated exogenous peptide, polypeptide, or protein) is enclosed by the lipid membrane structure, e.g., such an encapsulated moiety is located in the lumen of the enclosed lipid membrane structure (e.g., the lumen of a PMP). The encapsulated moiety (e.g., the encapsulated peptide, polypeptide, or protein) may, in some instances, interact or associate with the inner face of the lipid membrane structure. The exogenous peptide, polypeptide, or protein may, in some instances, be intercalated with the lipid membrane structure. In some instances, the exogenous peptide, polypeptide, or protein has an extraluminal portion. Alternatively, the term “encapsulate” or “encapsulated” may be used in the context of using a complex lipid particle to enclose a moiety (e.g., an exogenous peptide, polypeptide, or protein as defined herein) within the complex lipid particle. In some instances, “encapsulate” may be used in the context of using a modified PMP to enclose a moiety (e.g., an exogenous peptide, polypeptide, or protein as defined herein).

As used herein, the term “exogenous peptide, polypeptide, or protein” refers to a peptide, polypeptide, or protein (as is defined herein) that is encapsulated by a complex lipid particle or by a modified PMP (e.g., a PMP derived from a plant extracellular vesicle, and modified with one or more exogenous lipids) that does not naturally occur in a plant lipid vesicle (e.g., does not naturally occur in a plant extracellular vesicle) or that is encapsulated in a complex lipid particle or a modified PMP in an amount not found in a naturally occurring plant extracellular vesicle. The exogenous peptide, polypeptide, or protein may, in some instances, naturally occur in the plant from which the plant lipids are extracted or from which the PMP is derived. In other instances, the exogenous peptide, polypeptide, or protein does not naturally occur in the plant from which the plant lipids are extracted or from which the PMP is derived. The exogenous peptide, polypeptide, or protein may be artificially expressed in the plant from which the plant lipids are extracted or from which the PMP is derived, e.g., may be a heterologous polypeptide. The exogenous peptide, polypeptide, or protein may be derived from another organism. In some embodiments, the exogenous peptide, polypeptide, or protein is loaded into the complex lipid particles or the modified PMP formulation, e.g., using one or more of sonication, electroporation, lipid extraction, and lipid extrusion. The exogenous peptide, polypeptide, or protein may be, e.g., a therapeutic agent, an enzyme (e.g., a recombination enzyme or an editing enzyme), a hormone (e.g., insulin), a receptor agonist or a receptor antagonist (e.g., GLP-1 agonist, such as exenatide, semaglutide, or tirzepatide), or a pathogen control agent.

As used herein, “delivering” or “contacting” refers to providing or applying a complex lipid particle formulation or a modified PMP formulation (e.g., a modified PMP formulation comprising an exogenous protein or peptide) to an organism, e.g., an animal. Delivery to an animal may be, e.g., oral or enteral delivery (e.g., delivery by feeding or into the GI tract, e.g., by gavage) or systemic delivery (e.g., delivery by injection). The modified PMP formulation may be delivered to the digestive tract, e.g., the stomach, the small intestine, or the large intestine. The complex lipid particle formulation or modified PMP formulation may be stable in the digestive tract.

As used herein, the term “animal” refers to humans and non-human animals (including for example, dogs, cats, horses, rabbits, zoo animals, cows, pigs, sheep, chickens, and non-human primates).

As used herein, the term “formulated for delivery to an animal” refers to a complex lipid particle formulation or a modified PMP formulation that includes a pharmaceutically acceptable carrier.

As used herein, the term “infection” refers to the presence or colonization of a pathogen in an animal (e.g., in one or more parts of the animal), on an animal (e.g., on one or more parts of the animal), or in the habitat surrounding an animal, particularly where the infection decreases the fitness of the animal, e.g., by causing a disease, disease symptoms, or an immune (e.g., inflammatory) response.

As used herein the term “pathogen” refers to an organism, such as a microorganism or an invertebrate, which causes disease or disease symptoms in an animal by, e.g., (i) directly infecting the animal, (ii) producing agents that causes disease or disease symptoms in an animal (e.g., bacteria that produce pathogenic toxins and the like), and/or (iii) by eliciting an immune (e.g., inflammatory response) in animals (e.g., biting insects, e.g., bedbugs). As used herein, pathogens include, but are not limited to, bacteria, protozoa, parasites, fungi, nematodes, insects, viroids and viruses, or any combination thereof, wherein each pathogen is capable, either by itself or in concert with another pathogen, of eliciting disease or symptoms in humans.

As used herein, the term “polypeptide,” “peptide,” or “protein” encompasses any chain of naturally or non-naturally occurring amino acids (either D- or L-amino acids), regardless of length (e.g., at least 2, 3, 4, 5, 6, 7, 10, 12, 14, 16, 18, 20, 25, 30, 40, 50, 100, 150, 200, 250, 300, 350, 400, 450, 500, 550, 600, 650, 700, 750, 800, 850, 900, 950, 1000, or more than 1000 amino acids), the presence or absence of post-translational modifications (e.g., glycosylation or phosphorylation), or the presence of, e.g., one or more non-amino acyl groups (for example, sugar, lipid, etc.) covalently linked to the polypeptide, and includes, for example, natural polypeptides, synthetic or recombinant polypeptides, hybrid molecules, peptoids, or peptidomimetics. The polypeptide may be, e.g., at least 0.1, at least 1, at least 5, at least 10, at least 15, at least 20, at least 30, at least 40, at least 50, or more than 50 kD in size. The polypeptide may be a full-length protein. Alternatively, the polypeptide may comprise one or more domains of a protein.

As used herein, the term “antibody” encompasses an immunoglobulin, whether natural or partly or wholly synthetically produced, and fragments thereof, capable of specifically binding to an antigen. The term also covers any protein having a binding domain which is homologous to an immunoglobulin binding domain. These proteins can be derived from natural sources, or partly or wholly synthetically produced. “Antibody” further includes a polypeptide comprising a framework region from an immunoglobulin gene or fragments thereof that specifically binds and recognizes an antigen. Use of the term “antibody” is meant to include whole antibodies; polyclonal, monoclonal and recombinant antibodies; fragments thereof; and further includes single-chain antibodies (nanobodies); humanized antibodies; murine antibodies; chimeric, mouse-human, mouse-primate, primate-human monoclonal antibodies; anti-idiotype antibodies; antibody fragments, such as, e.g., scFv, (scFv)2, Fab, Fab′, and F(ab′)2, F(ab1)2, Fv, dAb, and Fd fragments; diabodies; and antibody-related polypeptides. “Antibody” further includes bispecific antibodies and multispecific antibodies.

The term “antigen binding fragment”, as used herein, refers to fragments of an intact immunoglobulin, and any part of a polypeptide including antigen binding regions having the ability to specifically bind to the antigen. For example, the antigen binding fragment may be a F(ab′)2 fragment, a Fab′ fragment, a Fab fragment, a Fv fragment, or a scFv fragment, but is not limited thereto. A Fab fragment has one antigen binding site and contains the variable regions of a light chain and a heavy chain, the constant region of the light chain, and the first constant region CH₁ of the heavy chain. A Fab′ fragment differs from a Fab fragment in that the Fab′ fragment additionally includes the hinge region of the heavy chain, including at least one cysteine residue at the C-terminal of the heavy chain CH₁ region. The F(ab′)2 fragment is produced whereby cysteine residues of the Fab′ fragment are joined by a disulfide bond at the hinge region. A Fv fragment is the minimal antibody fragment having only heavy chain variable regions and light chain variable regions, and a recombinant technique for producing the Fv fragment is well known in the art. Two-chain Fv fragments may have a structure in which heavy chain variable regions are linked to light chain variable regions by a non-covalent bond. Single-chain Fv (scFv) fragments generally may have a dimer structure as in the two-chain Fv fragments in which heavy chain variable regions are covalently bound to light chain variable regions via a peptide linker or heavy and light chain variable regions are directly linked to each other at the C-terminal thereof. The antigen binding fragment may be obtained using a protease (for example, a whole antibody is digested with papain to obtain Fab fragments, and is digested with pepsin to obtain F(ab′)2 fragments), and may be prepared by a genetic recombinant technique. A dAb fragment consists of a VH domain.

Single-chain antibody molecules may comprise a polymer with a number of individual molecules, for example, dimer, trimer or other polymers.

As used herein, the term “heterologous” refers to an agent (e.g., a polypeptide) that is either (1) exogenous to the plant (e.g., originating from a source that is not the plant or plant part from which the PMP is produced) (e.g., an agent which is added to the PMP using loading approaches described herein) or (2) endogenous to the plant cell or tissue from which the PMP is produced, but present in the PMP (e.g., added to the PMP using loading approaches described herein, genetic engineering, as well as in vitro or in vivo approaches) at a concentration that is higher than that found in nature (e.g., higher than a concentration found in a naturally-occurring plant extracellular vesicle).

As used herein, “percent identity” between two sequences is determined by the BLAST 2.0 algorithm, which is described in Altschul et al., (1990) J. Mol. Biol. 215:403-410. Software for performing BLAST analyses is publicly available through the National Center for Biotechnology Information.

As used herein, the term “plant” refers to whole plants, plant organs, plant tissues, seeds, plant cells, seeds, and progeny of the same. Plant cells include, without limitation, cells from seeds, suspension cultures, embryos, meristematic regions, callus tissue, leaves, roots, shoots, gametophytes, sporophytes, pollen, and microspores. Plant parts include differentiated and undifferentiated tissues including, but not limited to the following: roots, stems, shoots, leaves, pollen, seeds, fruit, harvested produce, tumor tissue, and various forms of cells and culture (e.g., single cells, protoplasts, embryos, and callus tissue). The plant tissue may be in a plant or in a plant organ, tissue, or cell culture. In addition, a plant may be genetically engineered to produce a heterologous protein or RNA.

As used herein, the term “complex lipid particle” refers to a lipid particle that has a complexity characterized by comprising a wide variety of lipids, including lipids extracted from one or more plant sources. The complex lipid particle may comprise between 10% w/w and 99% w/w lipids derived from a lipid structure from one or more plant sources, e.g., may contain at least 10% w/w, at least 20% w/w, at least 30% w/w, at least 40% w/w, at least 50% w/w, at least 60% w/w, at least 70% w/w, at least 80% w/w, at least 90% w/w, at least 95% w/w, or about 99% w/w lipids derived from a lipid structure from one or more plant sources. The complex lipid particle may contain 5-1000 lipids extracted from one or more plant sources. The complex lipid particle may contain plant lipids from at least 5, at least 6, at least 7, at least 8, at least 9, or at least 10 different classes or sub-classes of lipids from the plant source. The complex lipid particle may comprise all or a fraction of the lipid species present in the lipid structure from the plant source, e.g., it may contain at least 10%, at least 20%, at least 30%, at least 40%, at least 50%, at least 60%, at least 70%, at least 80%, at least 90%, or virtually 100% of the lipid species present in the lipid structure from the plant source. The complex lipid particle may comprise reduced or minimized protein matter endogenous to the one or more plant sources, e.g., may contain 0% w/w, less than 1% w/w, less than 5% w/w, less than 10% w/w, less than 15% w/w, less than 20% w/w, less than 30% w/w, less than 40% w/w, or less than 50% w/w of the protein matter endogenous to the one or more plant sources. In some instances, the lipid bilayer of the complex lipid particle does not contain proteins.

The complex lipid particle may further comprise at least two exogenous lipids. The complex lipid particle may include at least 1% w/w, at least 2% w/w, at least 5% w/w, at least 10% w/w, at least 15% w/w, at least 20% w/w, at least 25% w/w, at least 30% w/w, at least 40% w/w, at least 50% w/w, at least 60% w/w, at least 70% w/w, at least 80% w/w, or about 90% w/w exogenous lipids. Exemplary exogenous lipids include sterols and PEG-lipid conjugate. The complex lipid particle may be used to encapsulate an exogenous peptide, polypeptide, or protein, to enable delivery of the exogenous peptide, polypeptide, or protein to a target cell or tissue.

As used herein, the term “plant extracellular vesicle”, “plant EV”, or “EV” refers to an enclosed lipid-bilayer structure naturally occurring in a plant. Optionally, the plant EV includes one or more plant EV markers. As used herein, the term “plant EV marker” refers to a component that is naturally associated with a plant, such as a plant protein, a plant nucleic acid, a plant small molecule, a plant lipid, or a combination thereof, including but not limited to any of the plant EV markers listed in the Appendix disclosed in WO 2021/041301, which is incorporated herein by reference in its entirety. In some instances, the plant EV marker is an identifying marker of a plant EV but is not a pesticidal agent. In some instances, the plant EV marker is an identifying marker of a plant EV and also a pesticidal agent (e.g., either associated with or encapsulated by the plurality of PMPs, or not directly associated with or encapsulated by the plurality of PMPs).

As used herein, the term “plant messenger pack” or “PMP” refers to a lipid structure (e.g., a lipid bilayer, unilamellar, multilamellar structure; e.g., a vesicular lipid structure), that is about 5-2000 nm (e.g., at least 5-1000 nm, at least 5-500 nm, at least 400-500 nm, at least 25-250 nm, at least 50-150 nm, or at least 70-120 nm) in diameter that is derived from (e.g., enriched, isolated or purified from) a plant source or segment, portion, or extract thereof, including lipid or non-lipid components (e.g., peptides, nucleic acids, or small molecules) associated therewith and that has been enriched, isolated or purified from a plant, a plant part, or a plant cell, the enrichment or isolation removing one or more contaminants or undesired components from the source plant. PMPs may be highly purified preparations of naturally occurring EVs. Preferably, at least 1% of contaminants or undesired components from the source plant are removed (e.g., at least 2%, 5%, 10%, 15%, 20%, 25%, 30%, 40%, 45%, 50%, 55%, 60%, 70%, 80%, 90%, 95%, 96%, 98%, 99%, or 100%) of one or more contaminants or undesired components from the source plant, e.g., plant cell wall components; pectin; plant organelles (e.g., mitochondria; plastids such as chloroplasts, leucoplasts or amyloplasts; and nuclei); plant chromatin (e.g., a plant chromosome); or plant molecular aggregates (e.g., protein aggregates, protein-nucleic acid aggregates, lipoprotein aggregates, or lipido-proteic structures). Preferably, a PMP is at least 30% pure (e.g., at least 40% pure, at least 50% pure, at least 60% pure, at least 70% pure, at least 80% pure, at least 90% pure, at least 99% pure, or 100% pure) relative to the one or more contaminants or undesired components from the source plant as measured by weight (w/w), spectral imaging (% transmittance), or conductivity (S/m).

The PMPs may be modified to include exogenous lipids, e.g., lipids that are either (1) exogenous to the plant (e.g., originating from a source that is not the plant or plant part from which the PMP is produced) (e.g., added the PMP using methods described herein) or (2) endogenous to the plant cell or tissue from which the PMP is produced, but present in the PMP (e.g., added to the PMP using methods described herein, genetic engineering, in vitro or in vivo approaches) at a concentration that is higher than that found in nature (e.g., higher than a concentration found in a naturally-occurring plant extracellular vesicle). The lipid composition of the PMP may include 0%, less than 1%, or at least 1%, 2%, 5%, 10%, 15%, 20%, 25%, 30%, 40%, 50%, 60%, 70%, 80%, 90%, 95%, or more than 95% exogenous lipid. Exemplary exogenous lipids include cationic lipids, ionizable lipids, zwitterionic lipids, and lipidoids.

The PMPs may be modified to optionally include additional agents, such as polypeptides (e.g., peptides or proteins), therapeutic agents, polynucleotides, or small molecules. The PMPs can carry or associate with additional agents (e.g., polypeptides) in a variety of ways to enable delivery of the agent to a target plant, e.g., by encapsulating the agent, incorporation of the agent in the lipid bilayer structure, or association of the agent (e.g., by conjugation) with the surface of the lipid bilayer structure. Heterologous functional agents can be incorporated into the PMPs either in vivo (e.g., in planta) or in vitro (e.g., in tissue culture, in cell culture, or synthetically incorporated).

As used herein, the term “pure” refers to a PMP preparation in which at least a portion (e.g., at least 20%, 25%, 30%, 40%, 45%, 50%, 55%, 60%, 70%, 80%, 90%, 95%, 96%, 98%, 99%, or 100%) of plant cell wall components, plant organelles (e.g., mitochondria, chloroplasts, and nuclei), or plant molecule aggregates (protein aggregates, protein-nucleic acid aggregates, lipoprotein aggregates, or lipido-proteic structures) have been removed relative to the initial sample isolated from a plant, or part thereof.

As used herein, the term “exogenous lipid” refers to a lipid that is exogenous to the plant, i.e., a lipid originates from a source that is not the plant source from which the lipids are extracted (e.g., a lipid that is added to the complex lipid particle formulation using method described herein). The term “exogenous lipid” does not exclude a plant-derived lipid (such as a plant-derived sterol). That is to say, an exogenous lipid can be a plant-derived lipid (such as a plant-derived sterol that is exogenous to the plant source from which the lipids are extracted, e.g., an exogenous lipid can be a plant derived sterol that is added to the complex lipid particle formulation). An exogenous lipid may be a cell-penetrating agent, may be capable of increasing delivery of a peptide, polypeptide, or protein by the complex lipid formulation to a cell, and/or may be capable of increasing loading (e.g., loading efficiency or loading capacity) of a peptide, polypeptide, or protein. In some embodiments, the exogenous lipid may be a stabilizing lipid. In some embodiments, the exogenous lipid may be a structural lipid. Exemplary exogenous lipids include sterols and PEGylated lipids.

As used herein, the term “treatment” refers to administering a pharmaceutical composition to an animal for prophylactic and/or therapeutic purposes. To “prevent an infection” refers to prophylactic treatment of an animal that does not yet have a disease or condition, but which is susceptible to, or otherwise at risk of, a particular disease or condition. To “treat an infection” refers to administering treatment to an animal already suffering from a disease to improve or stabilize the animal's condition.

As used herein, the term “treat an infection” refers to administering treatment to an individual (e.g., an animal) already having a disease to improve or stabilize the individual's condition. This may involve reducing colonization of a pathogen in, on, or around an animal by one or more pathogens (e.g., by about 1%, 2%, 5%, 10%, 20%, 30%, 40%, 50%, 60%, 70%, 80%, 90%, or 100%) relative to a starting amount and/or allow benefit to the individual (e.g., reducing colonization in an amount sufficient to resolve symptoms). In such instances, a treated infection may manifest as a decrease in symptoms (e.g., by about 1%, 2%, 5%, 10%, 20%, 30%, 40%, 50%, 60%, 70%, 80%, 90%, or 100%). In some instances, a treated infection is effective to increase the likelihood of survival of an individual (e.g., an increase in likelihood of survival by about 1%, 2%, 5%, 10%, 20%, 30%, 40%, 50%, 60%, 70%, 80%, 90%, or 100%) or increase the overall survival of a population (e.g., an increase in likelihood of survival by about 1%, 2%, 5%, 10%, 20%, 30%, 40%, 50%, 60%, 70%, 80%, 90%, or 100%). For example, the compositions and methods may be effective to “substantially eliminate” an infection, which refers to a decrease in the infection in an amount sufficient to sustainably resolve symptoms (e.g., for at least 1, 2, 3, 4, 5, 6, 7, 8, 9, 10, 11, or 12 months) in the animal.

As used herein, the term “prevent an infection” refers to preventing an increase in colonization in, on, or around an animal by one or more pathogens (e.g., by about 1%, 2%, 5%, 10%, 20%, 30%, 40%, 50%, 60%, 70%, 80%, 90%, 100%, or more than 100% relative to an untreated animal) in an amount sufficient to maintain an initial pathogen population (e.g., approximately the amount found in a healthy individual), prevent the onset of an infection, and/or prevent symptoms or conditions associated with infection. For example, an individual (e.g., an animal, e.g., a human) may receive prophylaxis treatment to prevent a fungal infection while being prepared for an invasive medical procedure (e.g., preparing for surgery, such as receiving a transplant, stem cell therapy, a graft, a prosthesis, receiving long-term or frequent intravenous catheterization, or receiving treatment in an intensive care unit), in immunocompromised individuals (e.g., individuals with cancer, with HIV/AIDS, or taking immunosuppressive agents), or in individuals undergoing long term antibiotic therapy.

As used herein, the expression that the complex lipid formulation or the modified PMP formulation is “stable” refers to a complex lipid formulation or modified PMP composition that over a period of time (e.g., at least 24 hours, at least 48 hours, at least 1 week, at least 2 weeks, at least 3 weeks, at least 4 weeks, at least 30 days, at least 60 days, or at least 90 days) retains at least 5% (e.g., at least 5%, 10%, 15%, 20%, 25%, 30%, 35%, 40%, 45%, 50%, 55%, 60%, 65%, 70%, 75%, 80%, 85%, 90%, 95%, or 100%) of the initial number of complex lipid particles or modified PMPs (e.g., complex lipid particles or modified PMPs per mL of solution) relative to the number of complex lipid particles or modified PMPs in the complex lipid formulation or modified PMP composition (e.g., at the time of production or formulation) optionally at a defined temperature range (e.g., a temperature of at least 24° C. (e.g., at least 24° C., 25° C., 26° C., 27° C., 28° C., 29° C., or 30° C.), at least 20° C. (e.g., at least 20° C., 21° C., 22° C., or 23° C.), at least 4° C. (e.g., at least 5° C., 10° C., or 15° C.), at least −20° C. (e.g., at least −20° C., −15° C., −10° C., −5° C., or 0° C.), or −80° C. (e.g., at least −80° C., −70° C., −60° C., −50° C., −40° C., or −30° C.).

Alternatively, the expression refers to a complex lipid formulation or the modified PMP composition that over a period of time (e.g., at least 24 hours, at least 48 hours, at least 1 week, at least 2 weeks, at least 3 weeks, at least 4 weeks, at least 30 days, at least 60 days, or at least 90 days) retains at least 5% (e.g., at least 5%, 10%, 15%, 20%, 25%, 30%, 35%, 40%, 45%, 50%, 55%, 60%, 65%, 70%, 75%, 80%, 85%, 90%, 95%, or 100%) of its activity relative to the initial activity of the complex lipid formulation or the modified PMP formulation (e.g., at the time of production or formulation) optionally at a defined temperature range (e.g., a temperature of at least 24° C. (e.g., at least 24° C., 25° C., 26° C., 27° C., 28° C., 29° C., or 30° C.), at least 20° C. (e.g., at least 20° C., 21° C., 22° C., or 23° C.), at least 4° C. (e.g., at least 5° C., 10° C., or 15° C.), at least −20° C. (e.g., at least −20° C., −15° C., −10° C., −5° C., or 0° C.), or −80° C. (e.g., at least −80° C., −70° C., −60° C., −50° C., −40° C., or −30° C.)).

Alternatively, the expression refers to a complex lipid formulation or a modified PMP formulation that over a period of time (e.g., at least 24 hours, at least 48 hours, at least 1 week, at least 2 weeks, at least 3 weeks, at least 4 weeks, at least 30 days, at least 60 days, or at least 90 days) retains their particle size, i.e., the particle size does not increase, or has an increase of no more than 5% (e.g., no more than 5%, 10%, 15%, 20%, 25%, 30%, 35%, 40%, 45%, 50%, 55%, 60%, 65%, 70%, 75%, 80%, 85%, 90%, 95%, 100%, 150%, 2-fold, 2.5-fold, or 3-fold) relative to the initial size of the complex lipid particles or modified PMPs (e.g., at the time of production or formulation) optionally at a defined temperature range (e.g., a temperature of at least 24° C. (e.g., at least 24° C., 25° C., 26° C., 27° C., 28° C., 29° C., or 30° C.), at least 20° C. (e.g., at least 20° C., 21° C., 22° C., or 23° C.), at least 4° C. (e.g., at least 5° C., 10° C., or 15° C.), at least −20° C. (e.g., at least −20° C., −15° C., −10° C., −5° C., or 0° C.), or −80° C. (e.g., at least −80° C., −70° C., −60° C., −50° C., −40° C., or −30° C.)).

In some embodiments, the stable complex lipid formulation or modified PMP continues to encapsulate or remains associated with an exogenous peptide, polypeptide, or protein with which the complex lipid formulation or modified PMP has been loaded, e.g., continues to encapsulate or remains associated with an exogenous peptide, polypeptide, or protein for at least 24 hours, at least 48 hours, at least 1 week, at least 2 weeks, at least 3 weeks, at least 4 weeks, at least 30 days, at least 60 days, at least 90 days, or 90 or more days.

As used herein, the term “vector” refers to an insect that can carry or transmit an animal pathogen from a reservoir to an animal. Exemplary vectors include insects, such as those with piercing-sucking mouthparts, as found in Hemiptera and some Hymenoptera and Diptera such as mosquitoes, bees, wasps, midges, lice, tsetse fly, fleas and ants, as well as members of the Arachnidae such as ticks and mites.

As used herein, the term “juice sac” or “juice vesicle” refers to a juice-containing membrane-bound component of the endocarp (carpel) of a hesperidium, e.g., a citrus fruit. In some embodiments, the juice sacs are separated from other portions of the fruit, e.g., the rind (exocarp or flavedo), the inner rind (mesocarp, albedo, or pith), the central column (placenta), the segment walls, or the seeds. In some embodiments, the juice sacs are juice sacs of a grapefruit, a lemon, a lime, or an orange.

### II. Complex Lipid Particle or Modified PMPs Encapsulating Polypeptide

One aspect of the invention relates to a complex lipid formulation, comprising a plurality of complex lipid particles, each complex lipid particle of the plurality comprising at least five lipids extracted from one or more plant sources and at least two exogenous lipids; and one or more exogenous peptides, polypeptides, or proteins, encapsulated in the complex lipid particles. The complex lipid particles are characterized by one or more of the following characteristics:
i) containing less than 50% w/w of protein matter endogenous to the one or more plant sources; and
ii) containing less than 50 mol % of ionizable lipids.

Another aspect of the invention relates to plant messenger packs (PMPs) modified with one or more exogenous lipids.

The complex lipid formulation or modified PMP formulation described herein includes an exogenous peptide, polypeptide, or protein, e.g., an exogenous peptide, polypeptide, or protein described in Section III herein. A plurality of complex lipid particles or modified PMPs may be loaded with the exogenous peptide, polypeptide, or protein such that at least 5%, at least 10%, at least 15%, at least 25%, at least 50%, at least 75%, at least 90%, or at least 95% of the complex lipid particles or modified PMPs encapsulate the exogenous peptide, polypeptide, or protein.

The exogenous peptide, polypeptide, or protein may be, e.g., a therapeutic agent, a pathogen control agent (e.g., an agent having antipathogen activity (e.g., antibacterial, antifungal, antinematicidal, antiparasitic, or antiviral activity)), or an enzyme (e.g., a recombination enzyme or an editing enzyme.

### A. CLPs

Complex lipid particles (CLPs) described herein comprise a wide variety of lipids extracted from one or more plant sources. The complex lipid particle may comprise between 10% w/w and 99% w/w lipids derived from a lipid structure from one or more plant sources, e.g., may contain at least 10% w/w, at least 20% w/w, at least 30% w/w, at least 40% w/w, at least 50% w/w, at least 60% w/w, at least 70% w/w, at least 80% w/w, at least 90% w/w, at least 95% w/w, or about 99% w/w lipids derived from a lipid structure from one or more plant sources.

In some embodiments, the complex lipid particle comprises about 10-95% w/w of the plant lipids. For instance, the complex lipid particle comprises about 25-95% w/w, about 30-95% w/w, about 35-95% w/w, about 40-95% w/w, about 45-95% w/w, about 50-95% w/w, about 55-95% w/w, about 60-95% w/w, about 65-95% w/w, about 70-95% w/w, about 75-95% w/w, about 80-95% w/w, or about 85-95% w/w of the plant lipids based on the amounts of total lipids in the complex lipid formulation.

The complex lipid particle may contain 5-1000 lipids extracted from one or more plant sources. In some embodiments, the complex lipid particle contains at least 10 plant lipids belonging to one or more of the classes selected from the group consisting of glycerolipid, sphingolipid, and sterol. For instance, the complex lipid particle contains at least 15, at least 20, at least 25, at least 30, at least 35, at least 40, at least 45, at least 50, at least 60, at least 70, at least 80, at least 90, at least 100, at least 150, at least 200, at least 250, at least 300, at least 400, at least 500, at least 600, at least 700, or at least 800 plant lipids belonging to one or more of the classes selected from the group consisting of glycerolipid, sphingolipid, and sterol. In some embodiments, the complex lipid particle contains lipids from at least two or at least three of these different classes.

The complex lipid particle may contain one or more glycerolipids selected from the group consisting of phospholipids (PL), galactolipids (GL), triacylglycerols (TG), and sulfolipids (SL). In some embodiments, the complex lipid particle contains one or more sphingolipids selected from the group consisting of glycosyl inositolphosphoceramides (GIPC), glucosylceramides (GCer), ceramides (Cer), and free long-chain bases (LCB). In some embodiments, the complex lipid particle contains one or more phytosterols selected from the group consisting of campesterol, stigmasterol, and sitosterol.

The complex lipid particle may contain one or more lipids belonging to one or more of the sub-classes selected from the group consisting of acyl diacylglyceryl glucuronides, acylhexosylceramides, acylsterylglycosides, bile acids, acyl carnitines, cholesteryl esters, ceramides, cardiolipins, coenzyme Qs, diacylglycerols, digalactosyldiacylglycerols, diacylglyceryl glucuronides, dilysocardiolipins, fatty acids, fatty acid esters of hydroxyl fatty acids, hemibismonoacylglycerophosphates, hexosylceramides, lysophosphatidic acids, lysophosphatidylcholines, lysophosphatidylethanolamines, N-acyl-lysophosphatidylethanolamines, lysophosphatidylglycerols, lysophosphatidylinositols, lysophosphatidylserines, monogalactosyldiacylglycerols, lysocardiolipins, N-acyl ethanolamines, N-acyl glycines, N-acyl glycyl serines, phosphatidic acids, phosphatidylcholines, phosphatidylethanolamines, phosphatidylethanols, phosphatidylglycerols, phosphatidylinositols, ceramide phosphoinositols, phosphatidylmethanols, phosphatidylserines, steryl esters, stigmasterols, sulfatides, sulfonolipids, sphingomyelins, sulfoquinovosyl diacylglycerols, sterols, and triacylglycerols. In some embodiments, the complex lipid particle contains at least 10 plant lipids belonging to one or more of the sub-classes selected from the group consisting of the sub-classes listed above. For instance, the complex lipid particle contains at least 15, at least 20, at least 25, at least 30, at least 35, at least 40, at least 45, at least 50, at least 60, at least 70, at least 80, at least 90, at least 100, at least 150, at least 200, at least 250, at least 300, at least 400, at least 500, at least 600, at least 700, or at least 800 plant lipids belonging to one or more of the sub-classes selected from the group consisting of the sub-classes listed above.

The complex lipid particle may contain 10 or more lipids belonging to one or more of the sub-classes selected from the group consisting of acylsterylglycosides, ceramides, digalactosyldiacylglycerols, diacylglyceryl glucuronides, hemibismonoacylglycerophosphates, hexosylceramides, lysophosphatidylcholines, lysophosphatidylethanolamines, monogalactosyldiacylglycerols, phosphatidylcholines, phosphatidylethanolamines, phosphatidylethanols, phosphatidylglycerols, phosphatidylinositols, sulfoquinovosyl diacylglycerols, and sterols. For instance, the complex lipid particle contains at least 15, at least 20, at least 25, at least 30, at least 35, at least 40, at least 45, at least 50, at least 60, at least 70, at least 80, at least 90, at least 100, at least 150, at least 200, at least 250, at least 300, at least 400, at least 400, at least 500, at least 600, at least 700, or at least 800 plant lipids belonging to one or more of the sub-classes selected from the group consisting of the sub-classes listed above.

The complex lipid particle may contain plant lipids from at least 5, at least 6, at least 7, at least 8, at least 9, or at least 10 different classes or sub-classes of lipids from the plant source.

The identity (and class and subclass) and the amounts of the lipids extracted from the plant source can be analyzed by lipidomics analysis by solubilizing the lipid extracts or complex lipid particles in compatible solvents and analyzing by a mass spectrometry (e.g., MS/MS). An example of MS/MS based lipidomics analysis of the complex lipid particles is shown in Example 2.

The complex lipid particle may comprise all or a fraction of the lipid species present in the lipid structure from the plant source, e.g., it may contain at least 10%, at least 20%, at least 30%, at least 40%, at least 50%, at least 60%, at least 70%, at least 80%, at least 90%, or virtually 100% of the lipid species present in the lipid structure from the plant source.

The complex lipid particle may comprise reduced or minimized protein matter endogenous to the one or more plant sources. For instance, the complex lipid particle may contain less than 50% w/w, less than 45% w/w, less than 40% w/w, less than 35% w/w, less than 30% w/w, less than 25% w/w, less than 20% w/w, less than 15% w/w, less than 10% w/w, less than 9% w/w, less than 8% w/w, less than 7% w/w, less than 6% w/w, less than 5% w/w, less than 4% w/w, less than 3% w/w, less than 2% w/w, less than 1% w/w, less than 0.5% w/w, less than 0.1% w/w, or essentially free of protein matter endogenous to the one or more plant sources. In some instances, the lipid bilayer of the complex lipid particle does not contain proteins. To calculate % w/w of residual protein matter endogenous to the one or more plant sources, protein concentration is divided by the concentration of the plant lipid extract and then multiplied by 100. Alternatively, % w/w is calculated as the percent of the mass of total protein endogenous to the one or more plant sources based on the mass of the total lipid extract.

The complex lipid particle may comprise reduced or minimized residual dsDNA matter endogenous to the one or more plant sources. For instance, the complex lipid particle may contain less than 15% w/w, less than 10% w/w, less than 5% w/w, less than 1% w/w, less than 0.5% w/w, less than 0.1% w/w, less than 0.05% w/w, less than 0.01% w/w, less than 0.005% w/w, less than 0.001% w/w, or essentially free of residual dsDNA matter endogenous to the one or more plant sources. In some instances, the lipid bilayer of the complex lipid particle does not contain residual dsDNA. To calculate % w/w of residual dsDNA matter endogenous to the one or more plant sources, total adjusted dsDNA concentration is divided by the concentration of the plant lipid extract and then multiplied by 100. Alternatively, % w/w is calculated as the percent of the mass of total residual dsDNA endogenous to the one or more plant sources based on the mass of the total lipid extract.

The complex lipid particle may further comprise at least two exogenous lipids. The complex lipid particle may include at least 1% w/w, at least 2% w/w, at least 5% w/w, at least 10% w/w, at least 15% w/w, at least 20% w/w, at least 25% w/w, at least 30% w/w, at least 40% w/w, at least 50% w/w, at least 60% w/w, at least 70% w/w, at least 80% w/w, or about 90% w/w exogenous lipids. For instance, the complex lipid particle may contain a sterol and PEG-lipid conjugate. Additional exogenous lipids suitable for being included in the complex lipid particle are described herein below.

### B. PMPs

PMPs can include plant EVs, or segments, portions, or extracts, thereof, in which the plant EVs are about 5-2000 nm in diameter. For example, the PMP can include a plant EV, or segment, portion, or extract thereof, that has a mean diameter of about 5-50 nm, about 50-100 nm, about 100-150 nm, about 150-200 nm, about 200-250 nm, about 250-300 nm, about 300-350 nm, about 350-400 nm, about 400-450 nm, about 450-500 nm, about 500-550 nm, about 550-600 nm, about 600-650 nm, about 650-700 nm, about 700-750 nm, about 750-800 nm, about 800-850 nm, about 850-900 nm, about 900-950 nm, about 950-1000 nm, about 1000-1250 nm, about 1250-1500 nm, about 1500-1750 nm, or about 1750-2000 nm. In some instances, the PMP includes a plant EV, or segment, portion, or extract thereof, that has a mean diameter of about 5-950 nm, about 5-900 nm, about 5-850 nm, about 5-800 nm, about 5-750 nm, about 5-700 nm, about 5-650 nm, about 5-600 nm, about 5-550 nm, about 5-500 nm, about 5-450 nm, about 5-400 nm, about 5-350 nm, about 5-300 nm, about 5-250 nm, about 5-200 nm, about 5-150 nm, about 5-100 nm, about 5-50 nm, or about 5-25 nm. In certain instances, the plant EV, or segment, portion, or extract thereof, has a mean diameter of about 50-200 nm. In certain instances, the plant EV, or segment, portion, or extract thereof, has a mean diameter of about 50-300 nm. In certain instances, the plant EV, or segment, portion, or extract thereof, has a mean diameter of about 200-500 nm. In certain instances, the plant EV, or segment, portion, or extract thereof, has a mean diameter of about 30-150 nm.

In some instances, the PMP may include a plant EV, or segment, portion, or extract thereof, that has a mean diameter of at least 5 nm, at least 50 nm, at least 100 nm, at least 150 nm, at least 200 nm, at least 250 nm, at least 300 nm, at least 350 nm, at least 400 nm, at least 450 nm, at least 500 nm, at least 550 nm, at least 600 nm, at least 650 nm, at least 700 nm, at least 750 nm, at least 800 nm, at least 850 nm, at least 900 nm, at least 950 nm, or at least 1000 nm. In some instances, the PMP includes a plant EV, or segment, portion, or extract thereof, that has a mean diameter less than 1000 nm, less than 950 nm, less than 900 nm, less than 850 nm, less than 800 nm, less than 750 nm, less than 700 nm, less than 650 nm, less than 600 nm, less than 550 nm, less than 500 nm, less than 450 nm, less than 400 nm, less than 350 nm, less than 300 nm, less than 250 nm, less than 200 nm, less than 150 nm, less than 100 nm, or less than 50 nm. A variety of methods (e.g., a dynamic light scattering method) standard in the art can be used to measure the particle diameter of the plant EVs, or segment, portion, or extract thereof.

In some instances, the PMP may include a plant EV, or segment, portion, or extract thereof, that has a mean surface area of 77 nm² to 3.2×10⁶ nm² (e.g., 77-100 nm², 100-1000 nm², 1000-1×10⁴ nm², 1×10⁴-1×10⁵ nm², 1×10⁵-1×10⁶ nm², or 1×10⁶-3.2×10⁶ nm²). In some instances, the PMP may include a plant EV, or segment, portion, or extract thereof, that has a mean volume of 65 nm³ to 5.3×10⁸ nm³ (e.g., 65-100 nm³, 100-1000 nm³, 1000-1×10⁴ nm³, 1×10⁴-1×10⁵ nm³, 1×10⁵-1×10⁶ nm³, 1×10⁶-1×10⁷ nm³, 1×10⁷-1×10⁸ nm³, 1×10⁸-5.3×10⁸ nm³). In some instances, the PMP may include a plant EV, or segment, portion, or extract thereof, that has a mean surface area of at least 77 nm², (e.g., at least 77 nm², at least 100 nm², at least 1000 nm², at least 1×10⁴ nm², at least 1×10⁵ nm², at least 1×10⁶ nm², or at least 2×10⁶ nm²). In some instances, the PMP may include a plant EV, or segment, portion, or extract thereof, that has a mean volume of at least 65 nm³ (e.g., at least 65 nm³, at least 100 nm³, at least 1000 nm³, at least 1×10⁴ nm³, at least 1×10⁵ nm³, at least 1×10⁶ nm³, at least 1×10⁷ nm³, at least 1×10⁸ nm³, at least 2×10⁸ nm³, at least 3×10⁸ nm³, at least 4×10⁸ nm³, or at least 5×10⁸ nm³.

In some instances, the PMP can have the same size as the plant EV or segment, extract, or portion thereof. Alternatively, the PMP may have a different size than the initial plant EV from which the PMP is produced. For example, the PMP may have a diameter of about 5-2000 nm in diameter. For example, the PMP can have a mean diameter of about 5-50 nm, about 50-100 nm, about 100-150 nm, about 150-200 nm, about 200-250 nm, about 250-300 nm, about 300-350 nm, about 350-400 nm, about 400-450 nm, about 450-500 nm, about 500-550 nm, about 550-600 nm, about 600-650 nm, about 650-700 nm, about 700-750 nm, about 750-800 nm, about 800-850 nm, about 850-900 nm, about 900-950 nm, about 950-1000 nm, about 1000-1200 nm, about 1200-1400 nm, about 1400-1600 nm, about 1600-1800 nm, or about 1800-2000 nm. In some instances, the PMP may have a mean diameter of at least 5 nm, at least 50 nm, at least 100 nm, at least 150 nm, at least 200 nm, at least 250 nm, at least 300 nm, at least 350 nm, at least 400 nm, at least 450 nm, at least 500 nm, at least 550 nm, at least 600 nm, at least 650 nm, at least 700 nm, at least 750 nm, at least 800 nm, at least 850 nm, at least 900 nm, at least 950 nm, at least 1000 nm, at least 1200 nm, at least 1400 nm, at least 1600 nm, at least 1800 nm, or about 2000 nm. A variety of methods (e.g., a dynamic light scattering method) standard in the art can be used to measure the particle diameter of the PMPs. In some instances, the size of the PMP is determined following loading of heterologous functional agents or following other modifications to the PMPs.

In some instances, the PMP may have a mean surface area of 77 nm² to 1.3×10⁷ nm² (e.g., 77-100 nm², 100-1000 nm², 1000-1×10⁴ nm², 1×10⁴-1×10⁵ nm², 1×10⁵-1×10⁶ nm², or 1×10⁶-1.3×10⁷ nm²). In some instances, the PMP may have a mean volume of 65 nm³ to 4.2×10⁹ nm³ (e.g., 65-100 nm³, 100-1000 nm³, 1000-1×10⁴ nm³, 1×10⁴-1×10⁵ nm³, 1×10⁵-1×10⁶ nm³, 1×10⁶-1×10⁷ nm³, 1×10⁷-1×10⁸ nm³, 1×10⁸-1×10⁹ nm³, or 1×10⁹-4.2×10⁹ nm³). In some instances, the PMP has a mean surface area of at least 77 nm², (e.g., at least 77 nm², at least 100 nm², at least 1000 nm², at least 1×10⁴ nm², at least 1×10⁵ nm², at least 1×10⁶ nm², or at least 1×10⁷ nm²). In some instances, the PMP has a mean volume of at least 65 nm³ (e.g., at least 65 nm³, at least 100 nm³, at least 1000 nm³, at least 1×10⁴ nm³, at least 1×10⁵ nm³, at least 1×10⁶ nm³, at least 1×10⁷ nm³, at least 1×10⁸ nm³, at least 1×10⁹ nm³, at least 2×10⁹ nm³, at least 3×10⁹ nm³, or at least 4×10⁹ nm³).

In some instances, the PMP may include an intact plant EV. Alternatively, the PMP may include a segment, portion, or extract of the full surface area of the vesicle (e.g., a segment, portion, or extract including less than 100% (e.g., less than 90%, less than 80%, less than 70%, less than 60%, less than 50%, less than 40%, less than 30%, less than 20%, less than 10%, less than 10%, less than 5%, or less than 1%) of the full surface area of the vesicle) of a plant EV. The segment, portion, or extract may be any shape, such as a circumferential segment, spherical segment (e.g., hemisphere), curvilinear segment, linear segment, or flat segment. In instances where the segment is a spherical segment of the vesicle, the spherical segment may represent one that arises from the splitting of a spherical vesicle along a pair of parallel lines, or one that arises from the splitting of a spherical vesicle along a pair of non-parallel lines. Accordingly, the plurality of PMPs can include a plurality of intact plant EVs, a plurality of plant EV segments, portions, or extracts, or a mixture of intact and segments of plant EVs. One skilled in the art will appreciate that the ratio of intact to segmented plant EVs will depend on the particular isolation method used. For example, grinding or blending a plant, or part thereof, may produce PMPs that contain a higher percentage of plant EV segments, portions, or extracts than a non-destructive extraction method, such as vacuum-infiltration.

In instances where the PMP includes a segment, portion, or extract of a plant EV, the EV segment, portion, or extract may have a mean surface area less than that of an intact vesicle, e.g., a mean surface area less than 77 nm², 100 nm², 1000 nm², 1×10⁴ nm², 1×10⁵ nm², 1×10⁶ nm², or 3.2×10⁶ nm²). In some instances, the EV segment, portion, or extract has a surface area of less than 70 nm², 60 nm², 50 nm², 40 nm², 30 nm², 20 nm², or 10 nm²). In some instances, the PMP may include a plant EV, or segment, portion, or extract thereof, that has a mean volume less than that of an intact vesicle, e.g., a mean volume of less than 65 nm³, 100 nm³, 1000 nm³, 1×10⁴ nm³, 1×10⁵ nm³, 1×10⁶ nm³, 1×10⁷ nm³, 1×10⁸ nm³, or 5.3×10⁸ nm³).

In instances where the PMP includes an extract of a plant EV, e.g., in instances where the PMP includes lipids extracted (e.g., with chloroform) from a plant EV, the PMP may include at least 1%, 2%, 5%, 10%, 20%, 30%, 40%, 50%, 60%, 70%, 80%, 90%, 95%, 99%, or more than 99% of lipids extracted (e.g., with chloroform) from a plant EV. The PMPs in the plurality may include plant EV segments and/or plant EV-extracted lipids or a mixture thereof.

### C. Production Methods of PMPs

PMPs may be produced from plant EVs, or a segment, portion or extract (e.g., lipid extract) thereof, that occur naturally in plants, or parts thereof, including plant tissues or plant cells. An exemplary method for producing PMPs includes (a) providing an initial sample from a plant, or a part thereof, wherein the plant or part thereof comprises EVs; and (b) isolating a crude PMP fraction from the initial sample, wherein the crude PMP fraction has a decreased level of at least one contaminant or undesired component from the plant or part thereof relative to the level in the initial sample. The method can further include an additional step (c) comprising purifying the crude PMP fraction, thereby producing a plurality of pure PMPs, wherein the plurality of pure PMPs have a decreased level of at least one contaminant or undesired component from the plant or part thereof relative to the level in the crude EV fraction. Each production step is discussed in further detail, below. Exemplary methods regarding the isolation and purification of PMPs is found, for example, in Rutter and Innes, Plant Physiol. 173(1): 728-741, 2017; Rutter et al, Bio. Protoc. 7(17): e2533, 2017; Regente et al, J of Exp. Biol. 68(20): 5485-5496, 2017; Mu et al, Mol. Nutr. Food Res., 58, 1561-1573, 2014, and Regente et al, FEBS Letters. 583: 3363-3366, 2009, each of which is herein incorporated by reference.

For example, a plurality of PMPs may be isolated from a plant by a process which includes the steps of: (a) providing an initial sample from a plant, or a part thereof, wherein the plant or part thereof comprises EVs; (b) isolating a crude PMP fraction from the initial sample, wherein the crude PMP fraction has a decreased level of at least one contaminant or undesired component from the plant or part thereof relative to the level in the initial sample (e.g., a level that is decreased by at least 1%, 2%, 5%, 10%, 15%, 20%, 25%, 30%, 40%, 45%, 50%, 55%, 60%, 70%, 80%, 90%, 95%, 96%, 98%, 99%, or 100%); and (c) purifying the crude PMP fraction, thereby producing a plurality of pure PMPs, wherein the plurality of pure PMPs have a decreased level of at least one contaminant or undesired component from the plant or part thereof relative to the level in the crude EV fraction (e.g., a level that is decreased by at least 1%, 2%, 5%, 10%, 15%, 20%, 25%, 30%, 40%, 45%, 50%, 55%, 60%, 70%, 80%, 90%, 95%, 96%, 98%, 99%, or 100%).

The PMPs provided herein can include a plant EV, or segment, portion, or extract thereof, isolated from a variety of plants. PMPs may be isolated from any genera of plants (vascular or nonvascular), including, but not limited to, angiosperms (monocotyledonous and dicotyledonous plants), gymnosperms, ferns, selaginellas, horsetails, psilophytes, lycophytes, algae (e.g., unicellular or multicellular, e.g., archaeplastida), or bryophytes. In certain instances, PMPs can be produced from a vascular plant, for example monocotyledons or dicotyledons or gymnosperms. For example, PMPs can be produced from alfalfa, apple, Arabidopsis, banana, barley, canola, castor bean, chicory, chrysanthemum, clover, cocoa, coffee, cotton, cottonseed, corn, crambe, cranberry, cucumber, dendrobium, Dioscorea, eucalyptus, fescue, flax, gladiolus, liliacea, linseed, millet, muskmelon, mustard, oat, oil palm, oilseed rape, papaya, peanut, pineapple, ornamental plants, Phaseolus, potato, rapeseed, rice, rye, ryegrass, safflower, sesame, sorghum, soybean, sugarbeet, sugarcane, sunflower, strawberry, tobacco, tomato, turfgrass, wheat or vegetable crops such as lettuce, celery, broccoli, cauliflower, cucurbits; fruit and nut trees, such as apple, pear, peach, orange, grapefruit, lemon, lime, almond, pecan, walnut, hazel; vines, such as grapes, kiwi, hops; fruit shrubs and brambles, such as raspberry, blackberry, gooseberry; forest trees, such as ash, pine, fir, maple, oak, chestnut, popular; with alfalfa, canola, castor bean, corn, cotton, crambe, flax, linseed, mustard, oil palm, oilseed rape, peanut, potato, rice, safflower, sesame, soybean, sugarbeet, sunflower, tobacco, tomato, or wheat. In some embodiments, PMPs can be produced from dragon fruit, kale, spinach, or strawberry.

PMPs may be produced from a whole plant (e.g., whole rosettes or seedlings) or alternatively from one or more plant parts (e.g., leaf, seed, root, fruit, vegetable, pollen, phloem sap, or xylem sap). For example, PMPs can be produced from shoot vegetative organs/structures (e.g., leaves, stems, or tubers), roots, flowers and floral organs/structures (e.g., pollen, bracts, sepals, petals, stamens, carpels, anthers, or ovules), seed (including embryo, endosperm, or seed coat), fruit (the mature ovary), sap (e.g., phloem or xylem sap), plant tissue (e.g., vascular tissue, ground tissue, tumor tissue, or the like), and cells (e.g., single cells, protoplasts, embryos, callus tissue, guard cells, egg cells, or the like), or progeny of the same. For instance, the isolation step may involve (a) providing a plant, or a part thereof, wherein the plant part is an Arabidopsis leaf. The plant may be at any stage of development. For example, the PMP can be produced from seedlings, e.g., 1 week, 2 week, 3 week, 4 week, 5 week, 6 week, 7 week, or 8 week old seedlings (e.g., Arabidopsis seedlings). Other exemplary PMPs can include PMPs produced from roots (e.g., ginger roots), fruit juice (e.g., grapefruit juice), vegetables (e.g., broccoli), pollen (e.g., olive pollen), phloem sap (e.g., Arabidopsis phloem sap), or xylem sap (e.g., tomato plant xylem sap). In some embodiments, the PMP is produced from a citrus fruit, e.g., a grapefruit or a lemon.

PMPs can be produced from a plant, or part thereof, by a variety of methods. Any method that allows release of the EV-containing apoplastic fraction of a plant, or an otherwise extracellular fraction that contains PMPs comprising secreted EVs (e.g., cell culture media) is suitable in the present methods. EVs can be separated from the plant or plant part by either destructive (e.g., grinding or blending of a plant, or any plant part) or non-destructive (washing or vacuum infiltration of a plant or any plant part) methods. For instance, the plant, or part thereof, can be vacuum-infiltrated, ground, blended, or a combination thereof to isolate EVs from the plant or plant part, thereby producing PMPs. For instance, the isolating step may involve (b) isolating a crude PMP fraction from the initial sample (e.g., a plant, a plant part, or a sample derived from a plant or a plant part), wherein the crude PMP fraction has a decreased level of at least one contaminant or undesired component from the plant or part thereof relative to the level in the initial sample; wherein the isolating step involves vacuum infiltrating the plant (e.g., with a vesicle isolation buffer) to release and collect the apoplastic fraction. Alternatively, the isolating step may involve (b) grinding or blending the plant to release the EVs, thereby producing PMPs.

Upon isolating the plant EVs, thereby producing PMPs, the PMPs can be separated or collected into a crude PMP fraction (e.g., an apoplastic fraction). For instance, the separating step may involve separating the plurality of PMPs into a crude PMP fraction using centrifugation (e.g., differential centrifugation or ultracentrifugation) and/or filtration to separate the PMP-containing fraction from large contaminants, including plant tissue debris, plant cells, or plant cell organelles (e.g., nuclei or chloroplast). As such, the crude PMP fraction will have a decreased number of large contaminants, including, for example, plant tissue debris, plant cells, or plant cell organelles (e.g., nuclei, mitochondria or chloroplast), as compared to the initial sample from the source plant or plant part.

The crude PMP fraction can be further purified by additional purification methods to produce a plurality of pure PMPs. For example, the crude PMP fraction can be separated from other plant components by ultracentrifugation, e.g., using a density gradient (iodixanol or sucrose), size-exclusion, and/or use of other approaches to remove aggregated components (e.g., precipitation or size-exclusion chromatography). The resulting pure PMPs may have a decreased level of contaminants or undesired components from the source plant (e.g., one or more non-PMP components, such as protein aggregates, nucleic acid aggregates, protein-nucleic acid aggregates, free lipoproteins, or lipido-proteic structures), nuclei, cell wall components, cell organelles, or a combination thereof) relative to one or more fractions generated during the earlier separation steps, or relative to a pre-established threshold level, e.g., a commercial release specification. For example, the pure PMPs may have a decreased level (e.g., by about 5%, 10%, 15%, 20%, 30%, 40%, 50%, 60%, 70%, 80%, 90%, 100%, or more than 100%; or by about 2× fold, 4× fold, 5× fold, 10× fold, 20× fold, 25× fold, 50× fold, 75× fold, 100× fold, or more than 100× fold) of plant organelles or cell wall components relative to the level in the initial sample. In some instances, the pure PMPs are substantially free (e.g., have undetectable levels) of one or more non-PMP components, such as protein aggregates, nucleic acid aggregates, protein-nucleic acid aggregates, free lipoproteins, lipido-proteic structures), nuclei, cell wall components, cell organelles, or a combination thereof. Further examples of the releasing and separation steps can be found in WO 2021/041301, which is incorporated herein by reference in its entirety. The PMPs may be at a concentration of, e.g., 1×10⁹, 5×10⁹, 1×10¹⁰, 5×10¹⁰, 5×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, or more than 1×10¹³ PMPs/mL.

For example, protein aggregates may be removed from isolated PMPs. For example, the isolated PMP solution can be taken through a range of pHs (e.g., as measured using a pH probe) to precipitate out protein aggregates in solution. The pH can be adjusted to, e.g., pH 3, pH 5, pH 7, pH 9, or pH 11 with the addition of, e.g., sodium hydroxide or hydrochloric acid. Once the solution is at the specified pH, it can be filtered to remove particulates. Alternatively, the isolated PMP solution can be flocculated using the addition of charged polymers, such as Polymin-P or Praestol 2640. Briefly, Polymin-P or Praestol 2640 is added to the solution and mixed with an impeller. The solution can then be filtered to remove particulates. Alternatively, aggregates can be solubilized by increasing salt concentration. For example, NaCl can be added to the isolated PMP solution until it is at, e.g., 1 mol/L. The solution can then be filtered to isolate the PMPs. Alternatively, aggregates are solubilized by increasing the temperature. For example, the isolated PMPs can be heated under mixing until the solution has reached a uniform temperature of, e.g., 50° C. for 5 minutes. The PMP mixture can then be filtered to isolate the PMPs. Alternatively, soluble contaminants from PMP solutions can be separated by size-exclusion chromatography column according to standard procedures, where PMPs elute in the first fractions, whereas proteins and ribonucleoproteins and some lipoproteins are eluted later. The efficiency of protein aggregate removal can be determined by measuring and comparing the protein concentration before and after removal of protein aggregates via BCA/Bradford protein quantification. In some embodiments, protein aggregates are removed before the exogenous peptide, polypeptide, or protein is encapsulated by the PMP. In other embodiments, protein aggregates are removed after the exogenous peptide, polypeptide, or protein is encapsulated by the PMP.

Any of the production methods described herein can be supplemented with any quantitative or qualitative methods known in the art to characterize or identify the PMPs at any step of the production process. PMPs may be characterized by a variety of analysis methods to estimate PMP yield, PMP concentration, PMP purity, PMP composition, or PMP sizes. PMPs can be evaluated by a number of methods known in the art that enable visualization, quantitation, or qualitative characterization (e.g., identification of the composition) of the PMPs, such as microscopy (e.g., transmission electron microscopy), dynamic light scattering, nanoparticle tracking, spectroscopy (e.g., Fourier transform infrared analysis), or mass spectrometry (protein and lipid analysis). In certain instances, methods (e.g., mass spectroscopy) may be used to identify plant EV markers present on the PMP, such as markers disclosed in the Appendix disclosed in WO 2021/041301, which is incorporated herein by reference in its entirety. To aid in analysis and characterization of the PMP fraction, the PMPs can additionally be labelled or stained. For example, the PMPs can be stained with 3,3′-dihexyloxacarbocyanine iodide (DIOC₆), a fluorescent lipophilic dye, PKH67 (Sigma Aldrich), Alexa Fluor® 488 (Thermo Fisher Scientific), or DyLight™ 800 (Thermo Fisher). In the absence of sophisticated forms of nanoparticle tracking, this relatively simple approach quantifies the total membrane content and can be used to indirectly measure the concentration of PMPs (Rutter and Innes, Plant Physiol. 173(1): 728-741, 2017; Rutter et al, Bio. Protoc. 7(17): e2533, 2017). For more precise measurements, and to assess the size distributions of PMPs, nanoparticle tracking, nano flow cytometry, or Tunable Resistive Pulse Sensing can be used.

During the production process, the PMPs can optionally be prepared such that the PMPs are at an increased concentration (e.g., by about 5%, 10%, 15%, 20%, 30%, 40%, 50%, 60%, 70%, 80%, 90%, 100%, or more than 100%; or by about 2× fold, 4× fold, 5× fold, 10× fold, 20× fold, 25× fold, 50× fold, 75× fold, 100× fold, or more than 100× fold) relative to the EV level in a control or initial sample. The isolated PMPs may make up about 0.1% to about 100% of the PMP composition, such as any one of about 0.01% to about 100%, about 1% to about 99.9%, about 0.1% to about 10%, about 1% to about 25%, about 10% to about 50%, or about 50% to about 99%. In some instances, the composition includes at least any of 0.1%, 0.5%, 1%, 5%, 10%, 20%, 30%, 40%, 50%, 60%, 70%, 80%, 90%, 95%, or more PMPs, e.g., as measured by wt/vol, percent PMP protein composition, and/or percent lipid composition (e.g., by measuring fluorescently labelled lipids)). In some instances, the concentrated agents are used as commercial products, e.g., the final user may use diluted agents, which have a substantially lower concentration of active ingredient. In some embodiments, the composition is formulated as a PMP concentrate formulation, e.g., an ultra-low-volume concentrate formulation. In some embodiments, the PMPs in the composition are at a concentration effective to increase the fitness of an organism, e.g., a plant, an animal, an insect, a bacterium, or a fungus. In other aspects, the PMPs in the composition are at a concentration effective to decrease the fitness of an organism, e.g., a plant, an animal, an insect, a bacterium, or a fungus.

PMPs can be produced from a variety of plants, or parts thereof (e.g., the leaf apoplast, seed apoplast, root, fruit, vegetable, pollen, phloem, or xylem sap). For example, PMPs can be released from the apoplastic fraction of a plant, such as the apoplast of a leaf (e.g., apoplast Arabidopsis thaliana leaves) or the apoplast of seeds (e.g., apoplast of sunflower seeds). Other exemplary PMPs are produced from roots (e.g., ginger roots), fruit juice (e.g., grapefruit juice), vegetables (e.g., broccoli), pollen (e.g., olive pollen), phloem sap (e.g., Arabidopsis phloem sap), xylem sap (e.g., tomato plant xylem sap), or cell culture supernatant (e.g. BY2 tobacco cell culture supernatant). WO 2021/041301, which is incorporated by reference in its entirety, further demonstrates the production of PMPs from these various plant sources.

PMPs can be produced and purified by a variety of methods, for example, by using a density gradient (iodixanol or sucrose) in conjunction with ultracentrifugation and/or methods to remove aggregated contaminants, e.g., precipitation or size-exclusion chromatography. Further descriptions regarding the production, purification, and characterization of PMPs can be found in WO 2021/041301, which is incorporated by reference in its entirety.

In some instances, the PMPs of the present compositions and methods can be isolated from a plant, or part thereof, and used without further modification to the PMP. In other instances, the PMP can be modified prior to use, as outlined further herein.

### D. Plant EV-Markers

The PMPs may have a range of markers that identify the PMP as being produced from a plant EV, and/or including a segment, portion, or extract thereof. As used herein, the term “plant EV-marker” refers to a component that is naturally associated with a plant and incorporated into or onto the plant EV in planta, such as a plant protein, a plant nucleic acid, a plant small molecule, a plant lipid, or a combination thereof. Examples of plant EV-markers can be found, for example, in Rutter and Innes, Plant Physiol. 173(1): 728-741, 2017; Raimondo et al., Oncotarget. 6(23): 19514, 2015; Ju et al., Mol. Therapy. 21(7):1345-1357, 2013; Wang et al., Molecular Therapy. 22(3): 522-534, 2014; and Regente et al., J of Exp. Biol. 68(20): 5485-5496, 2017; each of which is incorporated herein by reference. Additional examples of plant EV-markers are listed in the Appendix disclosed in WO 2021/041301, which is incorporated herein by reference in its entirety, and are further outlined herein.

The plant EV marker can include a plant lipid. Examples of plant lipid markers that may be found in the PMP include phytosterol, campesterol, β-sitosterol, stigmasterol, avenasterol, glycosyl inositol phosphoryl ceramides (GIPCs), glycolipids (e.g., monogalactosyldiacylglycerol (MGDG) or digalactosyldiacylglycerol (DGDG)), or a combination thereof. For instance, the PMP may include GIPCs, which represent the main sphingolipid class in plants and are one of the most abundant membrane lipids in plants. Other plant EV markers may include lipids that accumulate in plants in response to abiotic or biotic stressors (e.g., bacterial or fungal infection), such as phosphatidic acid (PA) or phosphatidylinositol-4-phosphate (PI4P).

Alternatively, the plant EV marker may include a plant protein. In some instances, the protein plant EV marker may be an antimicrobial protein naturally produced by plants, including defense proteins that plants secrete in response to abiotic or biotic stressors (e.g., bacterial or fungal infection). Plant pathogen defense proteins include soluble N-ethylmaleimide-sensitive factor association protein receptor protein (SNARE) proteins (e.g., Syntaxin-121 (SYP121; GenBank Accession No.: NP_187788.1 or NP_974288.1), Penetration1 (PEN1; GenBank Accession No: NP_567462.1)) or ABC transporter Penetration3 (PEN3; GenBank Accession No: NP_191283.2). Other examples of plant EV markers include proteins that facilitate the long-distance transport of RNA in plants, including phloem proteins (e.g., Phloem protein2-A1 (PP2-A1), GenBank Accession No: NP_193719.1), calcium-dependent lipid-binding proteins, or lectins (e.g., Jacalin-related lectins, e.g., Helianthus annuus jacalin (Helja; GenBank: AHZ86978.1). For example, the RNA binding protein may be Glycine-Rich RNA Binding Protein-7 (GRP7; GenBank Accession Number: NP_179760.1). Additionally, proteins that regulate plasmodesmata function can in some instances be found in plant EVs, including proteins such as Synap-Totgamin A A (GenBank Accession No: NP_565495.1). In some instances, the plant EV marker can include a protein involved in lipid metabolism, such as phospholipase C or phospholipase D. In some instances, the plant protein EV marker is a cellular trafficking protein in plants. In certain instances where the plant EV marker is a protein, the protein marker may lack a signal peptide that is typically associated with secreted proteins. Unconventional secretory proteins seem to share several common features like (i) lack of a leader sequence, (ii) absence of PTMs specific for ER or Golgi apparatus, and/or (iii) secretion not affected by brefeldin A which blocks the classical ER/Golgi-dependent secretion pathway. One skilled in the art can use a variety of tools freely accessible to the public (e.g., SecretomeP Database; SUBA3 (SUBcellular localization database for Arabidopsis proteins)) to evaluate a protein for a signal sequence, or lack thereof.

In instances where the plant EV marker is a protein, the protein may have an amino acid sequence having at least 35%, 40%, 45%, 50%, 55%, 60%, 65%, 70%, 75%, 80%, 85%, 90%, 95%, 98%, 99%, or 100% sequence identity to a plant EV marker, such as any of the plant EV markers listed in the Appendix disclosed in WO 2021/041301, which is incorporated herein by reference in its entirety. For example, the protein may have an amino acid sequence having at least 35%, 40%, 45%, 50%, 55%, 60%, 65%, 70%, 75%, 80%, 85%, 90%, 95%, 98%, 99%, or 100% sequence identity to PEN1 from Arabidopsis thaliana (GenBank Accession Number: NP_567462.1).

In some instances, the plant EV marker includes a nucleic acid encoded in plants, e.g., a plant RNA, a plant DNA, or a plant PNA. For example, the PMP may include dsRNA, mRNA, a viral RNA, a microRNA (miRNA), or a small interfering RNA (siRNA) encoded by a plant. In some instances, the nucleic acid may be one that is associated with a protein that facilitates the long-distance transport of RNA in plants, as discussed herein. In some instances, the nucleic acid plant EV marker may be one involved in host-induced gene silencing (HIGS), which is the process by which plants silence foreign transcripts of plant pests (e.g., pathogens such as fungi). For example, the nucleic acid may be one that silences bacterial or fungal genes. In some instances, the nucleic acid may be a microRNA, such as miR159 or miR166, which target genes in a fungal pathogen (e.g., Verticillium dahliae). In some instances, the protein may be one involved in carrying plant defense compounds, such as proteins involved in glucosinolate (GSL) transport and metabolism, including Glucosinolate Transporter-1-1 (GTR1; GenBank Accession No: NP_566896.2), Glucosinolate Transporter-2 (GTR2; NP_201074.1), or Epithiospecific Modifier 1 (ESM1; NP_188037.1).

In instances where the plant EV marker is a nucleic acid, the nucleic acid may have a nucleotide sequence having at least 35%, 40%, 45%, 50%, 55%, 60%, 65%, 70%, 75%, 80%, 85%, 90%, 95%, 98%, 99%, or 100% sequence identity to a plant EV marker, e.g., such as those encoding the plant EV markers listed in the Appendix disclosed in WO 2021/041301, which is incorporated herein by reference in its entirety. For example, the nucleic acid may have a polynucleotide sequence having at least 35%, 40%, 45%, 50%, 55%, 60%, 65%, 70%, 75%, 80%, 85%, 90%, 95%, 98%, 99%, or 100% sequence identity to miR159 or miR166.

In some instances, the plant EV marker includes a compound produced by plants. For example, the compound may be a defense compound produced in response to abiotic or biotic stressors, such as secondary metabolites. One such secondary metabolite that be found in PMPs are glucosinolates (GSLs), which are nitrogen and sulfur-containing secondary metabolites found mainly in Brassicaceae plants. Other secondary metabolites may include allelochemicals.

In some instances, the PMP may also be identified as being produced from a plant EV based on the lack of certain markers (e.g., lipids, polypeptides, or polynucleotides) that are not typically produced by plants, but are generally associated with other organisms (e.g., markers of animal EVs, bacterial EVs, or fungal EVs). For example, in some instances, the PMP lacks lipids typically found in animal EVs, bacterial EVs, or fungal EVs. In some instances, the PMP lacks lipids typical of animal EVs (e.g., sphingomyelin). In some instances, the PMP does not contain lipids typical of bacterial EVs or bacterial membranes (e.g., LPS). In some instances, the PMP lacks lipids typical of fungal membranes (e.g., ergosterol).

Plant EV markers can be identified using any approaches known in the art that enable identification of small molecules (e.g., mass spectroscopy, mass spectrometry), lipids (e.g., mass spectroscopy, mass spectrometry), proteins (e.g., mass spectroscopy, immunoblotting), or nucleic acids (e.g., PCR analysis). In some instances, a PMP composition described herein includes a detectable amount, e.g., a pre-determined threshold amount, of a plant EV marker described herein.

### E. Exogenous Lipids

In some embodiments, the complex lipid particle comprises not only the lipids extracted from one or more plant sources, but also contains two or more exogenous lipids.

In some embodiments, the PMPs are modified to contain two or more exogenous lipids.

The exogenous lipid may be a cell-penetrating agent, may be capable of increasing delivery of a peptide, polypeptide, or protein by the complex lipid formulation to a cell, and/or may be capable of increasing loading (e.g., loading efficiency or loading capacity) of a peptide, polypeptide, or protein.

In some embodiments, the exogenous lipid may be a stabilizing lipid. In some embodiments, the exogenous lipid may be a structural lipid. Exemplary exogenous lipids include sterols and PEGylated lipids.

In some embodiments, the complex lipid particle includes other components (e.g., lipids, e.g., sterols, e.g., cholesterol; or small molecules).

In some embodiments, the PMPs can be modified with other components (e.g., lipids, e.g., sterols, e.g., cholesterol; or small molecules) to further alter the functional and structural characteristics of the PMP. For example, the PMPs can be further modified with stabilizing molecules that increase the stability of the PMPs (e.g., for at least one day at room temperature, and/or stable for at least one week at 4° C.).

In some embodiments, the complex lipid particle includes a sterol, e.g., sitosterol, sitostanol, β-sitosterol, 7α-hydroxycholesterol, pregnenolone, cholesterol (e.g., ovine cholesterol or cholesterol isolated from plants), stigmasterol, campesterol, fucosterol, or an analog (e.g., a glycoside, ester, or peptide) of any sterol.

In some embodiments, the PMP is modified with a sterol, e.g., sitosterol, sitostanol, β-sitosterol, 7α-hydroxycholesterol, pregnenolone, cholesterol (e.g., ovine cholesterol or cholesterol isolated from plants), stigmasterol, campesterol, fucosterol, or an analog (e.g., a glycoside, ester, or peptide) of any sterol.

In some examples, the exogenous sterol is added to the preparation prior to a mixing step, such as step (b), e.g., mixed with extracted lipids prior to step (b). The exogenous sterol may be added to amount to, e.g., 1%, 5%, 10%, 20%, 30%, 40%, 50%, 60%, 70%, 80%, 90%, or more than 90% (w/w) of total lipids and sterols in the preparation.

In some embodiments, the concentration of the sterol in the complex lipid particle ranges from about 5 to 60% w/w, for instance, from about 5 to 50% w/w, from about 5 to 40% w/w, from about 5 to 30% w/w, from about 5 to 20% w/w, from about 0.5 to 15% w/w, from about 0.5 to 15% w/w, from about 5 to 8% w/w, or from about 6 to 7% w/w, based on the amounts of total lipids in the complex lipid particle. In some embodiments, the sterol ranges from about 15 to 20% w/w, from about 20 to 30% w/w, from about 30 to 40% w/w, from about 40 to 50% w/w, or from about 50 to 60% w/w, based on the amounts of total lipids in the complex lipid particle.

In some embodiments, the concentration of the PEG-lipid conjugate ranges from about 0.5 to 5% w/w, from about 0.5 to 3.5% w/w, from about 1 to 3.5% w/w, from about 0.5 to 3% w/w, from about 1 to 3% w/w, from about 0.5 to 2.5% w/w, from about 1 to 2.5% w/w, from about 1.5 to 2.5% w/w, or from about 2 to 2.5% w/w, based on the amounts of total lipids in the complex lipid particle. In some embodiments, the concentration of the PEG-lipid conjugate ranges from about 0.5 to 15% w/w, from about 1 to 15% w/w, from about 2 to 5% w/w, from about 5 to 8% w/w, from about 8 to 12% w/w, or from about 12 to 15% w/w, based on the amounts of total lipids in the complex lipid particle.

In some embodiments, the sterol is cholesterol or sitosterol. In some instances, the complex lipid particle or the modified PMPs comprise a molar ratio of least 1%, 5%, 10%, 15%, 20%, 25%, 30%, 35%, 40%, 45%, 50%, 55%, 60%, or more than 60% sterol (e.g., cholesterol or sitosterol), e.g., 1%-10%, 10%-20%, 20%-30%, 30%-40%, 40%-50%, or 50%-60% sterol. In some embodiments, the complex lipid particle or the modified PMP comprises a molar ratio of about 35%-50% sterol (e.g., cholesterol or sitosterol), e.g., about 36%, 38.5%, 42.5%, or 46.5% sterol. In some embodiments, the complex lipid particle or the modified PMP comprises a molar ratio of about 20%-40% sterol.

In some embodiments, a PMP that has been modified with a sterol has altered stability (e.g., increased stability) relative to a PMP that has not been modified with a sterol. In some embodiments, a PMP that has been modified with a sterol has a greater rate of fusion with a membrane of a target cell relative to a PMP that has not been modified with a sterol.

In some instances, the complex lipid particle or the modified PMPs comprise an exogenous lipid and an exogenous sterol.

In some embodiments, the complex lipid particle comprises a PEGylated lipid.

In some embodiments, the PMP is modified with a PEGylated lipid.

Polyethylene glycol (PEG) length can vary from 1 kDa to 10 kDa; in some aspects, PEG having a length of 2 kDa is used. In some embodiments, the PEGylated lipid is C14-PEG2k, C18-PEG2k, or DMPE-PEG2k.

In some instances, the complex lipid particle or the modified PMPs comprise a molar ratio of at least 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.5%, 4%, 4.5%, 5%, 10%, 20%, 30%, 40%, 50%, or more than 50% PEGylated lipid (e.g., C14-PEG2k, C18-PEG2k, or DMPE-PEG2k), e.g., 0.1%-0.5%, 0.5%-1%, 1%-1.5%, 1.5%-2.5%, 2.5%-3.5%, 3.5%-5%, 5%-10%, 10%-20%, 20%-30%, 30%-40%, or 40%-50% PEGylated lipid. In some embodiments, the complex lipid particle or the modified PMP comprises a molar ratio of about about 0.1%-10% PEGylated lipid (e.g., C14-PEG2k, C18-PEG2k, or DMPE-PEG2k), e.g., about 1%-3% PEGylated lipid, e.g., about 1.5% or about 2.5% PEGylated lipid.

In some embodiments, a PMP that has been modified with a PEGylated lipid has altered stability (e.g., increased stability) relative to a PMP that has not been modified with a PEGylated lipid. In some embodiments, a PMP that has been modified with a PEGylated lipid has altered particle size relative to a PMP that has not been modified with a PEGylated lipid.

In some embodiments, a complex lipid particle or a modified PMP containing a PEGylated lipid is less likely to be phagocytosed than one containing no PEGylated lipid.

The addition of PEGylated lipids can also affect stability in GI tract and enhance particle migration through mucus. PEG may be used as a method to attach targeting moieties.

Cell uptake of the complex lipid particle or the modified PMPs can be measured by a variety of methods known in the art. For example, the complex lipid particle or the modified PMPs, or a component thereof, can be labelled with a marker (e.g., a fluorescent marker) that can be detected in isolated cells to confirm uptake.

The complex lipid particle contains less than 50 mol % of ionizable lipids (e.g., ionizable lipids exogenous to the one or more plant sources). For instance, the complex lipid particle contains less than 45 mol %, less than 40 mol %, less than 35 mol %, less than 30 mol %, less than 25 mol %, less than 20 mol %, less than 15 mol %, less than 10 mol %, less than 9 mol %, less than 8 mol %, less than 7 mol %, less than 6 mol %, less than 5 mol %, less than 4 mol %, less than 3 mol %, less than 2 mol %, less than 1 mol %, less than 0.5 mol %, less than 0.1 mol %, or essentially free of ionizable lipids (e.g., ionizable lipids exogenous to the one or more plant sources). In some embodiments, the complex lipid particle contains less than 20 mol % of exogenous ionizable lipids. In some embodiments, the complex lipid particle contains less than 5 mol % of exogenous ionizable lipids.

In some embodiments, a complex lipid formulation provided herein comprises two or more different types of complex lipid particles, e.g., comprises complex lipid particles derived from two or more different plant sources, and/or comprises complex lipid particles comprising different species and/or different ratios of exogenous lipids such as sterols, and/or PEGylated lipids.

In some embodiments, a modified PMP formulation provided herein comprises two or more different modified PMPs, e.g., comprises modified PMPs derived from different unmodified PMPs (e.g., unmodified PMPs from two or more different plant sources) and/or comprises modified PMPs comprising different species and/or different ratios of exogenous lipids such as sterols, and/or PEGylated lipids.

In some instances, the organic solvent in which the lipid film is dissolved is chloroform, ethanol, or dimethylformamide:methanol (DMF:MeOH). Alternatively, the organic solvent or solvent combination may be, e.g., acetonitrile, acetone, chloroform, ethanol, methanol, dimethylformamide, tetrahydrofuran, 1-butanol, dimethyl sulfoxide, acetonitrile:ethanol, acetonitrile:methanol, acetone:methanol, methyl tert-butyl ether:propanol, tetrahydrofuran:methanol, dimethyl sulfoxide:methanol, or dimethylformamide:methanol.

### F. Pharmaceutical Formulations

Included herein are complex lipid formulations or modified PMP formulations that can be formulated into pharmaceutical compositions, e.g., for administration to an animal, such as a human. The pharmaceutical composition may be administered to an animal with a pharmaceutically acceptable diluent, carrier, and/or excipient. Depending on the mode of administration and the dosage, the pharmaceutical composition of the methods described herein will be formulated into suitable pharmaceutical compositions to permit facile delivery. The single dose may be in a unit dose form as needed.

A complex lipid formulation or a modified PMP formulation may be formulated for e.g., oral administration, enteral administration, intravenous administration (e.g., injection or infusion), or subcutaneous administration to an animal (e.g., a human). For injectable formulations, various effective pharmaceutical carriers are known in the art (See, e.g., Remington: The Science and Practice of Pharmacy, 22^{nd} ed., (2012) and ASHP Handbook on Injectable Drugs, 18^{th} ed., (2014)).

Pharmaceutically acceptable carriers and excipients in the present compositions are nontoxic to recipients at the dosages and concentrations employed. Acceptable carriers and excipients may include buffers such as phosphate, citrate, HEPES, and TAE, antioxidants such as ascorbic acid and methionine, preservatives such as hexamethonium chloride, octadecyldimethylbenzyl ammonium chloride, resorcinol, and benzalkonium chloride, proteins such as human serum albumin, gelatin, dextran, and immunoglobulins, hydrophilic polymers such as polyvinylpyrrolidone, amino acids such as glycine, glutamine, histidine, and lysine, and carbohydrates such as glucose, mannose, sucrose, and sorbitol. The compositions may be formulated according to conventional pharmaceutical practice. The concentration of the compound in the formulation will vary depending upon a number of factors, including the dosage of the active agent (e.g., the exogenous peptide, polypeptide, or protein encapsulated by the complex lipid formulation or the modified PMP) to be administered, and the route of administration.

For oral administration to an animal, the complex lipid formulation or the modified PMP formulation can be prepared in the form of an oral formulation. Formulations for oral use can include tablets, caplets, capsules, syrups, or oral liquid dosage forms containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like. Formulations for oral use may also be provided in unit dosage form as chewable tablets, non-chewable tablets, caplets, capsules (e.g., as hard gelatin capsules wherein the active ingredient is mixed with an inert solid diluent, or as soft gelatin capsules wherein the active ingredient is mixed with water or an oil medium). The compositions disclosed herein may also further include an immediate-release, extended-release or delayed-release formulation.

For parenteral administration to an animal, the complex lipid formulation or the modified PMP compositions may be formulated in the form of liquid solutions or suspensions and administered by a parenteral route (e.g., topical, subcutaneous, intravenous, or intramuscular). The pharmaceutical composition can be formulated for injection or infusion. Pharmaceutical compositions for parenteral administration can be formulated using a sterile solution or any pharmaceutically acceptable liquid as a vehicle. Pharmaceutically acceptable vehicles include, but are not limited to, sterile water, physiological saline, or cell culture media (e.g., Dulbecco's Modified Eagle Medium (DMEM), α-Modified Eagles Medium (α-MEM), F-12 medium). Formulation methods are known in the art, see e.g., Gibson (ed.) Pharmaceutical Preformulation and Formulation (2nd ed.) Taylor & Francis Group, CRC Press (2009).

### III. Exogenous Peptides, Polypeptides, or Proteins

The present invention includes complex lipid formulations or modified PMP formulations wherein the complex lipid particle or the modified PMP encapsulates an exogenous peptide, polypeptide, or protein. The exogenous peptide, polypeptide, or protein may be enclosed within the complex lipid particles or the modified PMP, e.g., located inside the lipid membrane structure, e.g., separated from the surrounding material or solution by both leaflets of a lipid bilayer. In some embodiments, the encapsulated exogenous peptide, polypeptide, or protein may interact or associate with the inner lipid membrane of the complex lipid particle or the modified PMP. In some embodiments, the encapsulated exogenous peptide, polypeptide, or protein may interact or associate with the outer lipid membrane of the complex lipid particles or the modified PMP. The exogenous peptide, polypeptide, or protein may, in some instances, be intercalated with the lipid membrane structure. In some instances, the exogenous peptide, polypeptide, or protein has an extraluminal portion. In some instances, the exogenous peptide, polypeptide, or protein is conjugated to the outer surface of the lipid membrane structure, e.g., using click chemistry.

The exogenous peptide, polypeptide, or protein may be one that does not naturally occur in a plant EV. Alternatively, the exogenous peptide, polypeptide, or protein may be naturally occurring in a plant EV, but that is encapsulated in a complex lipid particle or a modified PMP in an amount not found in a naturally occurring plant extracellular vesicle. The exogenous peptide, polypeptide, or protein may, in some instances, naturally occur in the plant from which the plant lipids are extracted or from which the PMP is derived. In other instances, the exogenous peptide, polypeptide, or protein does not naturally occur in the plant from which the plant lipids are extracted or from which the PMP is derived. The exogenous polypeptide may be artificially expressed in the plant from which the plant lipids are extracted or from which the PMP is derived, e.g., may be a heterologous polypeptide. The exogenous peptide, polypeptide, or protein may be derived from another organism. In some embodiments, the exogenous peptide, polypeptide, or protein is loaded into the complex lipid particles or the modified PMP, e.g., using one or more of sonication, electroporation, lipid extraction, and lipid extrusion.

Peptides, polypeptides, or proteins included herein may include naturally occurring ones or recombinantly produced variants. In some instances, they may be functional fragments or variants thereof (e.g., an enzymatically active fragment or variant thereof). For example, the peptide, polypeptide, or protein may be a functionally active variant of any of the peptides, polypeptides, or proteins described herein with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region or over the entire sequence, to a sequence thereof described herein or a naturally occurring peptide, polypeptide, or protein. In some instances, the peptide, polypeptide, or protein may have at least 50% (e.g., at least 50%, 60%, 70%, 80%, 90%, 95%, 97%, 99%, or greater) identity to a peptide, polypeptide, or protein of interest.

The peptides, polypeptides, or proteins described herein may be formulated in a composition for any of the uses described herein. The compositions disclosed herein may include any number or type (e.g., classes) of peptides, polypeptides, or proteins, such as at least about any one of 1, 2, 3, 4, 5, 10, 15, 20, or more. A suitable concentration of each peptide, polypeptide, or protein in the composition depends on factors such as efficacy, stability of the peptide, polypeptide, or protein, number of distinct species in the composition, the formulation, and methods of application of the composition. In some instances, each peptide, polypeptide, or protein in a liquid composition is from about 0.1 ng/mL to about 100 mg/mL. In some instances, each peptide, polypeptide, or protein in a solid composition is from about 0.1 ng/g to about 100 mg/g.

Methods of making a peptide, polypeptide, or protein are routine in the art. See, in general, Smales & James (Eds.), Therapeutic Proteins: Methods and Protocols (Methods in Molecular Biology), Humana Press (2005); and Crommelin, Sindelar & Meibohm (Eds.), Pharmaceutical Biotechnology: Fundamentals and Applications, Springer (2013).

Methods for producing a peptide, polypeptide, or protein involve expression in plant cells, although recombinant proteins can also be produced using insect cells, yeast, bacteria, mammalian cells, or other cells under the control of appropriate promoters. Mammalian expression vectors may comprise nontranscribed elements such as an origin of replication, a suitable promoter and enhancer, and other 5′ or 3′ flanking nontranscribed sequences, and 5′ or 3′ nontranslated sequences such as necessary ribosome binding sites, a polyadenylation site, splice donor and acceptor sites, and termination sequences. DNA sequences derived from the SV40 viral genome, for example, SV40 origin, early promoter, enhancer, splice, and polyadenylation sites may be used to provide the other genetic elements required for expression of a heterologous DNA sequence. Appropriate cloning and expression vectors for use with bacterial, fungal, yeast, and mammalian cellular hosts are described in Green & Sambrook, Molecular Cloning: A Laboratory Manual (Fourth Edition), Cold Spring Harbor Laboratory Press (2012).

Various mammalian cell culture systems can be employed to express and manufacture a recombinant polypeptide agent. Examples of mammalian expression systems include CHO cells, COS cells, HeLA and BHK cell lines. Processes of host cell culture for production of protein therapeutics are described in, e.g., Zhou and Kantardjieff (Eds.), Mammalian Cell Cultures for Biologics Manufacturing (Advances in Biochemical Engineering/Biotechnology), Springer (2014). Purification of proteins is described in Franks, Protein Biotechnology: Isolation, Characterization, and Stabilization, Humana Press (2013); and in Cutler, Protein Purification Protocols (Methods in Molecular Biology), Humana Press (2010). Formulation of protein therapeutics is described in Meyer (Ed.), Therapeutic Protein Drug Products: Practical Approaches to formulation in the Laboratory, Manufacturing, and the Clinic, Woodhead Publishing Series (2012). Alternatively, the peptide, polypeptide, or protein may be a chemically synthesized one.

In some instances, the complex lipid formulation or the modified PMP includes an antibody or antigen binding fragment thereof. For example, an agent described herein may be an antibody that blocks or potentiates activity and/or function of a component of the pathogen. The antibody may act as an antagonist or agonist of a polypeptide (e.g., enzyme or cell receptor) in the pathogen. The making and use of antibodies against a target antigen in a pathogen is known in the art. See, for example, Zhiqiang An (Ed.), Therapeutic Monoclonal Antibodies: From Bench to Clinic, 1st Edition, Wiley, 2009 and also Greenfield (Ed.), Antibodies: A Laboratory Manual, 2nd Edition, Cold Spring Harbor Laboratory Press, 2013, for methods of making recombinant antibodies, including antibody engineering, use of degenerate oligonucleotides, 5′-RACE, phage display, and mutagenesis; antibody testing and characterization; antibody pharmacokinetics and pharmacodynamics; antibody purification and storage; and screening and labeling techniques.

The exogenous peptide, polypeptide, or protein may be released from the complex lipid formulation or the modified PMP in the target cell. In some embodiments, the exogenous peptide, polypeptide, or protein exerts activity in the cytoplasm of the target cell or in the nucleus of the target cell. The exogenous peptide, polypeptide, or protein may be translocated to the nucleus of the target cell.

In some embodiments, uptake by a cell of the exogenous peptide, polypeptide, or protein encapsulated by the complex lipid particle or the modified PMP is increased relative to uptake of the exogenous peptide, polypeptide, or protein not encapsulated by a complex lipid particle or modified PMP.

In some embodiments, the effectiveness of the exogenous polypeptide, or protein encapsulated by the complex lipid particle or the modified PMP is increased relative to the effectiveness of the exogenous peptide, polypeptide, or protein not encapsulated by a complex lipid particle or a modified PMP.

### A. Therapeutic Agents

The exogenous peptide, polypeptide, or protein may be a therapeutic agent, e.g., an agent used for the prevention or treatment of a condition or a disease. In some embodiments, the disease is a cancer, an autoimmune condition, or a metabolic disorder.

In some examples, the therapeutic agent is a peptide (e.g., a naturally occurring peptide, a recombinant peptide, or a synthetic peptide) or a protein (e.g., a naturally occurring protein, a recombinant protein, or a synthetic protein). In some examples, the protein is a fusion protein.

In some examples, the peptide, polypeptide, or protein is endogenous to the organism (e.g., mammal) to which the complex lipid formulation or the modified PMP is delivered. In other examples, the peptide, polypeptide, or protein is not endogenous to the organism.

In some examples, the therapeutic agent is an antibody (e.g., a monoclonal antibody, e.g., a monospecific, bispecific, or multispecific monoclonal antibody) or an antigen-binding fragment thereof (e.g., an scFv, (scFv)2, Fab, Fab′, and F(ab′)2, F(ab1)2, Fv, dAb, and Fd fragment, or a diabody), a nanobody, a conjugated antibody, or an antibody-related polypeptide.

In some examples, the therapeutic agent is an antimicrobial, antibacterial, antifungal, antinematicidal, antiparasitic, or antiviral polypeptide.

In some examples, the therapeutic agent is an allergenic, an allergen, or an antigen.

In some examples, the therapeutic agent is a vaccine (e.g., a conjugate vaccine, an inactivated vaccine, or a live attenuated vaccine),

In some examples, the therapeutic agent is an enzyme, e.g., a metabolic recombinase, a helicase, an integrase, a RNAse, a DNAse, a ubiquitination protein. In some examples, the enzyme is a recombinant enzyme.

In some examples, the therapeutic agent is a gene editing protein, e.g., a component of a CRISPR-Cas system, TALEN, or zinc finger.

In some examples, the therapeutic agent is any one of a cytokine, a hormone, a signaling ligand, a transcription factor, a receptor, a receptor antagonist, a receptor agonist, a blocking or neutralizing polypeptide, a riboprotein, or a chaperone.

In some examples, the therapeutic agent is a pore-forming protein, a cell-penetrating peptide, a cell-penetrating peptide inhibitor, or a proteolysis targeting chimera (PROTAC).

In some examples, the therapeutic agent is any one of an aptamer, a blood derivative, a cell therapy, or an immunotherapy (e.g., a cellular immunotherapy.

In some embodiments, the therapeutic agent is a protein or peptide therapeutic with enzymatic activity, regulatory activity, or targeting activity, e.g., a protein or peptide with activity that affects one or more of endocrine and growth regulation, metabolic enzyme deficiencies, hematopoiesis, hemostasis and thrombosis; gastrointestinal-tract disorders; pulmonary disorders; immunodeficiencies and/or immunoregulation; fertility; aging (e.g., anti-aging activity); autophagy regulation; epigenetic regulation; oncology; or infectious diseases (e.g., anti-microbial peptides, anti-fungals, or anti-virals).

In some embodiments, the therapeutic agent is a protein vaccine, e.g., a vaccine for use in protecting against a deleterious foreign agent, treating an autoimmune disease, or treating cancer (e.g., a neoantigen).

In some examples, the peptide, polypeptide, or protein is globular, fibrous, or disordered.

In some examples, the peptide, polypeptide, or protein has a size of less than 1, less than 2, less than 5, less than 10, less than 15, less than 20, less than 30, less than 40, less than 50, less than 60, less than 70, less than 80, less than 90, or less than 100 kD, e.g., has a size of 1-50 kD (e.g., 1-10, 10-20, 20-30, 30-40, or 40-50 kD) or 50-100 kD (e.g., 50-60, 60-70, 70-80, 80-90, or 90-100 kD).

In some examples, the peptide, polypeptide, or protein has an overall charge that is positive, negative, or neutral. The peptide, polypeptide, or protein may be modified such that the overall charge is altered, e.g., modified by adding one or more charged amino acids, for example, one or more (for example, 1-10 or 5-10) positively or negatively charged amino acids, such as an arginine tail (e.g., 5-10 arginine residues) to the N-terminus or C-terminus of the peptide, polypeptide, or protein.

In some embodiments, the disease is diabetes, e.g., diabetes mellitus, e.g., Type 1 diabetes mellitus. In some embodiments, diabetes is treated by administering to a patient an effective amount of a composition comprising a plurality of complex lipid particles or the modified PMPs, wherein one or more exogenous peptides, polypeptides, or proteins are encapsulated by the complex lipid particles or the modified PMP. In some embodiments, the administration of the plurality of complex lipid particles or modified PMPs lowers the blood sugar of the subject. In some embodiments, the therapeutic agent is insulin. In some embodiments, the therapeutic agent is exenatide, semaglutide, or tirzepatide.

In some examples, the therapeutic agent is an antibody shown in Table 1, a peptide shown in Table 2, an enzyme shown in Table 3, or a protein shown in Table 4.

TABLE 1
Antibodies

| Broad class | Molecule Type | Drug Name |
|---|---|---|
| Antibody | Monoclonal Antibody | 1D-09C3 |
| Antibody | Monoclonal Antibody Conjugated | 212 Pb-TCMC-Trastuzumab |
| Antibody | Monoclonal Antibody | 2141 V-11 |
| Antibody | Monoclonal Antibody | 3BNC-117 |
| Antibody | Monoclonal Antibody | 3BNC-117LS |
| Antibody | Monoclonal Antibody | 8H-9 |
| Antibody | Monoclonal Antibody Conjugated | A-166 |
| Antibody | Bispecific Monoclonal Antibody | A-337 |
| Antibody | Monoclonal Antibody | AB-011 |
| Antibody | Monoclonal Antibody | AB-022 |
| Antibody | Monoclonal Antibody | AB-023 |
| Antibody | Monoclonal Antibody | AB-154 |
| Antibody | Monoclonal Antibody | abagovomab |
| Antibody | Monoclonal Antibody Conjugated | ABBV-011 |
| Antibody | Monoclonal Antibody | ABBV-0805 |
| Antibody | Monoclonal Antibody Conjugated | ABBV-085 |
| Antibody | Monoclonal Antibody | ABBV-151 |
| Antibody | Monoclonal Antibody Conjugated | ABBV-155 |
| Antibody | Bispecific Monoclonal Antibody | ABBV-184 |
| Antibody | Monoclonal Antibody Conjugated | ABBV-321 |
| Antibody | Monoclonal Antibody Conjugated | ABBV-3373 |
| Antibody | Monoclonal Antibody | ABBV-368 |
| Antibody | Monoclonal Antibody | ABBV-927 |
| Antibody | Monoclonal Antibody | abciximab |
| Antibody | Monoclonal Antibody | abelacimab [INN] |
| Antibody | Monoclonal Antibody Conjugated | AbGn-107 |
| Antibody | Monoclonal Antibody | AbGn-168H |
| Antibody | Monoclonal Antibody | abituzumab |
| Antibody | Monoclonal Antibody | ACT-017 |
| Antibody | Monoclonal Antibody Conjugated | Actimab-A |
| Antibody | Monoclonal Antibody Conjugated | Actimab-M |
| Antibody | Cellular Immunotherapy; Gene | ACTR-087 + SEA-BCMA |
| | Therapy; Monoclonal Antibody | |
| Antibody | Cellular Immunotherapy; Gene | ACTR-707 |
| | Therapy; Monoclonal Antibody | |
| Antibody | Monoclonal Antibody | adalimumab |
| Antibody | Monoclonal Antibody | adalimumab biosimilar |
| Antibody | Monoclonal Antibody; Small Molecule | adavosertib + durvalumab |
| Antibody | Monoclonal Antibody Conjugated | ADCT-602 |
| Antibody | Antibody | adder [Vipera bents] |
| | | antivenom |
| Antibody | Monoclonal Antibody | ADG-106 |
| Antibody | Monoclonal Antibody | ADG-116 |
| Antibody | Monoclonal Antibody | adrecizumab |
| Antibody | Monoclonal Antibody | aducanumab |
| Antibody | Monoclonal Antibody | Aerucin |
| Antibody | Bispecific Monoclonal Antibody | AFM-13 |
| Antibody | Monoclonal Antibody | AGEN-1181 |
| Antibody | Monoclonal Antibody | AGEN-2373 |
| Antibody | Monoclonal Antibody Conjugated | AGS-16C3F |
| Antibody | Monoclonal Antibody | AGS-1C4D4 |
| Antibody | Monoclonal Antibody Conjugated | AGS-62P1 |
| Antibody | Monoclonal Antibody | AHM |
| Antibody | Monoclonal Antibody | AIMab-7195 |
| Antibody | Monoclonal Antibody | AK-002 |
| Antibody | Monoclonal Antibody | AK-101 |
| Antibody | Bispecific Monoclonal Antibody | AK-104 |
| Antibody | Monoclonal Antibody | AK-111 |
| Antibody | Bispecific Monoclonal Antibody | AK-112 |
| Antibody | Monoclonal Antibody | AL-001 |
| Antibody | Monoclonal Antibody | AL-002 |
| Antibody | Monoclonal Antibody | AL-003 |
| Antibody | Monoclonal Antibody | AL-101 |
| Antibody | Monoclonal Antibody | alemtuzumab |
| Antibody | Monoclonal Antibody | alirocumab |
| Antibody | Monoclonal Antibody Conjugated | ALTP-7 |
| Antibody | Bispecific Monoclonal Antibody | ALXN-1720 |
| Antibody | Antibody | AMAG-423 |
| Antibody | Monoclonal Antibody | amatuximab |
| Antibody | Bispecific Monoclonal Antibody | AMG-160 |
| Antibody | Bispecific Monoclonal Antibody | AMG-211 |
| Antibody | Monoclonal Antibody Conjugated | AMG-224 |
| Antibody | Monoclonal Antibody | AMG-301 |
| Antibody | Bispecific Monoclonal Antibody | AMG-330 |
| Antibody | Monoclonal Antibody | AMG-404 |
| Antibody | Bispecific Monoclonal Antibody | AMG-420 |
| Antibody | Bispecific Monoclonal Antibody | AMG-424 |
| Antibody | Bispecific Monoclonal Antibody | AMG-427 |
| Antibody | Bispecific Monoclonal Antibody | AMG-509 |
| Antibody | Monoclonal Antibody | AMG-529 |
| Antibody | Bispecific Monoclonal Antibody | AMG-673 |
| Antibody | Bispecific Monoclonal Antibody | AMG-701 |
| Antibody | Monoclonal Antibody | AMG-714 |
| Antibody | Bispecific Monoclonal Antibody | AMG-757 |
| Antibody | Monoclonal Antibody | AMG-820 |
| Antibody | Bispecific Monoclonal Antibody | AMV-564 |
| Antibody | Monoclonal Antibody | ANB-019 |
| Antibody | Monoclonal Antibody | andecaliximab |
| Antibody | Monoclonal Antibody Conjugated | anetumab ravtansine |
| Antibody | Monoclonal Antibody | anifrolumab |
| Antibody | Antibody | anthrax immune globulin |
| | | (human) |
| Antibody | Antibody | anti-thymocyte globulin |
| | | (equine) |
| Antibody | Antibody | anti-thymocyte globulin |
| | | (rabbit) |
| Antibody | Antibody | antivenin latrodectus equine |
| | | immune F(ab)2 |
| Antibody | Monoclonal Antibody | ANX-005 |
| Antibody | Monoclonal Antibody | ANX-007 |
| Antibody | Monoclonal Antibody | AP-101 |
| Antibody | Monoclonal Antibody | apitegromab |
| Antibody | Monoclonal Antibody | APL-501 |
| Antibody | Monoclonal Antibody | APL-502 |
| Antibody | Bispecific Monoclonal Antibody | APVO-436 |
| Antibody | Monoclonal Antibody | APX-003 |
| Antibody | Monoclonal Antibody | APX-005M |
| Antibody | Monoclonal Antibody | ARGX-109 |
| Antibody | Monoclonal Antibody | ARP-1536 |
| Antibody | Monoclonal Antibody Conjugated | ARX-788 |
| Antibody | Monoclonal Antibody | ascrinvacumab |
| Antibody | Monoclonal Antibody | ASLAN-004 |
| Antibody | Monoclonal Antibody | ASP-1650 |
| Antibody | Monoclonal Antibody | ASP-6294 |
| Antibody | Monoclonal Antibody | ASP-8374 |
| Antibody | Monoclonal Antibody | AT-1501 |
| Antibody | Monoclonal Antibody | atezolizumab |
| Antibody | Monoclonal Antibody | ATI-355 |
| Antibody | Monoclonal Antibody Conjugated | ATL-101 |
| Antibody | Bispecific Monoclonal Antibody | ATOR-1015 |
| Antibody | Monoclonal Antibody | ATOR-1017 |
| Antibody | Monoclonal Antibody | ATRC-101 |
| Antibody | Monoclonal Antibody | Atrosab |
| Antibody | Monoclonal Antibody Conjugated | Aurixim |
| Antibody | Monoclonal Antibody | AV-1 |
| Antibody | Monoclonal Antibody | avelumab |
| Antibody | Monoclonal Antibody Conjugated | AVID-100 |
| Antibody | Monoclonal Antibody Conjugated | AVID-200 |
| Antibody | Monoclonal Antibody | axatilimab |
| Antibody | Monoclonal Antibody | B-001 |
| Antibody | Monoclonal Antibody | balstilimab |
| Antibody | Monoclonal Antibody | basiliximab |
| Antibody | Monoclonal Antibody | BAT-4406 |
| Antibody | Monoclonal Antibody | batoclimab |
| Antibody | Monoclonal Antibody | bavituximab |
| Antibody | Monoclonal Antibody | BAY-1093884 |
| Antibody | Monoclonal Antibody | BAY-1834942 |
| Antibody | Monoclonal Antibody | BAY-1905254 |
| Antibody | Monoclonal Antibody Conjugated | BAY-2287411 |
| Antibody | Monoclonal Antibody Conjugated | BAY-2315497 |
| Antibody | Monoclonal Antibody Conjugated | BB-1701 |
| Antibody | Monoclonal Antibody Conjugated | BC-8SA |
| Antibody | Monoclonal Antibody Conjugated | BC-8Y90 |
| Antibody | Monoclonal Antibody | BCBA-445 |
| Antibody | Monoclonal Antibody | BCD-089 |
| Antibody | Monoclonal Antibody | BCD-096 |
| Antibody | Bispecific Monoclonal Antibody | BCD-121 |
| Antibody | Monoclonal Antibody | BCD-132 |
| Antibody | Monoclonal Antibody | BCD-145 |
| Antibody | Monoclonal Antibody | BCD-217 |
| Antibody | Monoclonal Antibody | begelomab |
| Antibody | Monoclonal Antibody Conjugated | belantamab mafodotin |
| Antibody | Monoclonal Antibody | belimumab |
| Antibody | Monoclonal Antibody | bemarituzumab |
| Antibody | Monoclonal Antibody | benralizumab |
| Antibody | Monoclonal Antibody | bentracimab |
| Antibody | Monoclonal Antibody | bermekimab |
| Antibody | Monoclonal Antibody | bertilimumab |
| Antibody | Monoclonal Antibody Conjugated | Betalutin |
| Antibody | Monoclonal Antibody | bevacizumab |
| Antibody | Monoclonal Antibody | bevacizumab biosimilar |
| Antibody | Monoclonal Antibody | bezlotoxumab |
| Antibody | Monoclonal Antibody | BG-00011 |
| Antibody | Monoclonal Antibody | BGB-149 |
| Antibody | Monoclonal Antibody | BHQ-880 |
| Antibody | Monoclonal Antibody | BI-1206 |
| Antibody | Monoclonal Antibody | BI-201 |
| Antibody | Monoclonal Antibody | BI-505 |
| Antibody | Monoclonal Antibody | BI-655064 |
| Antibody | Monoclonal Antibody | BI-655088 |
| Antibody | Monoclonal Antibody | BI-754091 |
| Antibody | Monoclonal Antibody | BI-754111 |
| Antibody | Monoclonal Antibody | BI-836826 |
| Antibody | Monoclonal Antibody | BI-836858 |
| Antibody | Bispecific Monoclonal Antibody | BI-836880 |
| Antibody | Monoclonal Antibody Conjugated | BIIB-015 |
| Antibody | Monoclonal Antibody | BIIB-059 |
| Antibody | Monoclonal Antibody | BIIB-076 |
| Antibody | Monoclonal Antibody | bimagrumab |
| Antibody | Monoclonal Antibody | bimekizumab |
| Antibody | Monoclonal Antibody | birtamimab |
| Antibody | Bispecific Monoclonal Antibody | Bispecific Monoclonal |
| | | Antibody to Agonize CD3 for |
| | | Acute Myelocytic Leukemia |
| Antibody | Bispecific Monoclonal Antibody | Bispecific Monoclonal |
| | | Antibody to Inhibit HIV 1 Env |
| | | for HIV Infections |
| Antibody | Bispecific Monoclonal Antibody | Bispecific Monoclonal |
| | | Antibody to Target CD3 and |
| | | FLT3 for Acute Myelocytic |
| | | Leukemia, Acute |
| | | Lymphocytic Leukemia and |
| | | Myelodysplastic Syndrome |
| Antibody | Bispecific Monoclonal Antibody | Bispecific Monoclonal |
| | | Antibody to Target GD2 and |
| | | CD3 for Oncology |
| Antibody | Bispecific Monoclonal Antibody | Bispecific Monoclonal |
| | | Antibody to Target PD-L1 |
| | | and CTLA4 for Pancreatic |
| | | Ductal Adenocarcinoma |
| Antibody | Monoclonal Antibody | BIVV-020 |
| Antibody | Monoclonal Antibody | BIW-8962 |
| Antibody | Antibody | black widow spider |
| | | [Latrodectus mactans] |
| | | antivenom [equine] |
| Antibody | Monoclonal Antibody | bleselumab |
| Antibody | Bispecific Monoclonal Antibody | blinatumomab |
| Antibody | Monoclonal Antibody Conjugated | BMS-936561 |
| Antibody | Monoclonal Antibody | BMS-986012 |
| Antibody | Monoclonal Antibody Conjugated | BMS-986148 |
| Antibody | Monoclonal Antibody | BMS-986156 |
| Antibody | Monoclonal Antibody | BMS-986178 |
| Antibody | Monoclonal Antibody | BMS-986179 |
| Antibody | Monoclonal Antibody | BMS-986207 |
| Antibody | Monoclonal Antibody | BMS-986218 |
| Antibody | Monoclonal Antibody | BMS-986226 |
| Antibody | Monoclonal Antibody | BMS-986253 |
| Antibody | Monoclonal Antibody | BMS-986258 |
| Antibody | Monoclonal Antibody | BNC-101 |
| Antibody | Monoclonal Antibody | BOS-161721 |
| Antibody | Antibody | botulism immune globulin |
| Antibody | Monoclonal Antibody | brazikumab |
| Antibody | Monoclonal Antibody Conjugated | brentuximab vedotin |
| Antibody | Monoclonal Antibody | BrevaRex MAb-AR20.5 |
| Antibody | Monoclonal Antibody | briakinumab |
| Antibody | Monoclonal Antibody | brodalumab |
| Antibody | Monoclonal Antibody | brolucizumab |
| Antibody | Monoclonal Antibody | BT-063 |
| Antibody | Antibody | BT-084 |
| Antibody | Antibody | BT-086 |
| Antibody | Antibody | BT-595 |
| Antibody | Monoclonal Antibody | BTI-322 |
| Antibody | Bispecific Monoclonal Antibody | BTRC-4017A |
| Antibody | Monoclonal Antibody | budigalimab |
| Antibody | Monoclonal Antibody | burosumab |
| Antibody | Monoclonal Antibody | BVX-20 |
| Antibody | Monoclonal Antibody | cabiralizumab |
| Antibody | Monoclonal Antibody | CAEL-101 |
| Antibody | Monoclonal Antibody | CAL |
| Antibody | Monoclonal Antibody Conjugated | camidanlumab tesirine |
| Antibody | Monoclonal Antibody | camrelizumab |
| Antibody | Monoclonal Antibody | canakinumab |
| Antibody | Monoclonal Antibody Conjugated | cantuzumab mertansine |
| Antibody | Monoclonal Antibody | caplacizumab |
| Antibody | Monoclonal Antibody | carotuximab |
| Antibody | Bispecific Monoclonal Antibody | catumaxomab |
| Antibody | Monoclonal Antibody | CBP-201 |
| Antibody | Bispecific Monoclonal Antibody | CC-1 |
| Antibody | Monoclonal Antibody | CC-90002 |
| Antibody | Monoclonal Antibody | CC-90006 |
| Antibody | Bispecific Monoclonal Antibody | CC-93269 |
| Antibody | Monoclonal Antibody Conjugated | CC-99712 |
| Antibody | Monoclonal Antibody Conjugated | CCW-702 |
| Antibody | Monoclonal Antibody | CDX-3379 |
| Antibody | Cellular Immunotherapy; Recombinant | Cellular Immunotherapy + |
| | Protein | edodekin alfa |
| Antibody | Monoclonal Antibody | cemiplimab |
| Antibody | Monoclonal Antibody | cendakimab |
| Antibody | Monoclonal Antibody | CERC-002 |
| Antibody | Monoclonal Antibody | CERC-007 |
| Antibody | Monoclonal Antibody | certolizumab pegol |
| Antibody | Monoclonal Antibody | certolizumab pegol |
| | | biosimilar |
| Antibody | Monoclonal Antibody | cetrelimab |
| Antibody | Monoclonal Antibody | cetuximab |
| Antibody | Monoclonal Antibody | cetuximab biosimilar |
| Antibody | Monoclonal Antibody Conjugated | cetuximab sarotalocan |
| Antibody | Monoclonal Antibody | CHOH-01 |
| Antibody | Bispecific Monoclonal Antibody | cibisatamab |
| Antibody | Monoclonal Antibody | cinpanemab |
| Antibody | Monoclonal Antibody | CIS-43 |
| Antibody | Monoclonal Antibody | CJM-112 |
| Antibody | Monoclonal Antibody | clazakizumab |
| Antibody | Monoclonal Antibody Conjugated | clivatuzumab tetraxetan |
| Antibody | Monoclonal Antibody | CM-101 |
| Antibody | Monoclonal Antibody | CNTO-6785 |
| Antibody | Monoclonal Antibody | codrituzumab |
| Antibody | Monoclonal Antibody Conjugated | cofetuzumab pelidotin |
| Antibody | Monoclonal Antibody | COM-701 |
| Antibody | Monoclonal Antibody | concizumab |
| Antibody | Monoclonal Antibody | COR-001 |
| Antibody | Antibody | coral snake [Micrurus] |
| | | (polyvalent) immunoglobulin |
| | | F (ab) 2 + Fab |
| | | immunoglobulin G |
| | | antivenom |
| Antibody | Monoclonal Antibody | cosibelimab |
| Antibody | Monoclonal Antibody | CPI-006 |
| Antibody | Monoclonal Antibody | crenezumab |
| Antibody | Monoclonal Antibody | crizanlizumab |
| Antibody | Monoclonal Antibody | crovalimab |
| Antibody | Monoclonal Antibody | CS-1001 |
| Antibody | Monoclonal Antibody | CS-1003 |
| Antibody | Monoclonal Antibody | CSL-311 |
| Antibody | Monoclonal Antibody | CSL-324 |
| Antibody | Monoclonal Antibody | CSL-346 |
| Antibody | Monoclonal Antibody | CSL-360 |
| Antibody | Monoclonal Antibody | CTX-471 |
| Antibody | Monoclonal Antibody | cusatuzumab |
| Antibody | Antibody | Cutaquig |
| Antibody | Antibody | Cuvitru |
| Antibody | Monoclonal Antibody | CX-072 |
| Antibody | Monoclonal Antibody Conjugated | CX-2009 |
| Antibody | Monoclonal Antibody Conjugated | CX-2029 |
| Antibody | Monoclonal Antibody | Cyto-111 |
| Antibody | Antibody | cytomegalovirus immune |
| | | globulin (human) |
| Antibody | Monoclonal Antibody; Small Molecule | dabrafenib mesylate + |
| | | panitumumab + trametinib |
| | | dimethyl sulfoxide |
| Antibody | Monoclonal Antibody | daclizumab |
| Antibody | Monoclonal Antibody | dalotuzumab |
| Antibody | Antisense Oligonucleotide; Monoclonal | danvatirsen + durvalumab |
| | Antibody | |
| Antibody | Monoclonal Antibody | dapirolizumab pegol |
| Antibody | Monoclonal Antibody | daratumumab |
| Antibody | Monoclonal Antibody | daxdilimab |
| Antibody | Monoclonal Antibody | DE-098 |
| Antibody | Antibody | death adder [Acanthophis |
| | | antarcticus] antivenom |
| | | [equine] |
| Antibody | Monoclonal Antibody | demcizumab |
| Antibody | Monoclonal Antibody | denosumab |
| Antibody | Monoclonal Antibody | denosumab biosimilar |
| Antibody | Monoclonal Antibody | depatuxizumab |
| Antibody | Monoclonal Antibody Conjugated | depatuxizumab mafodotin |
| Antibody | Monoclonal Antibody | dezamizumab |
| Antibody | Antibody | digoxin immune Fab (ovine) |
| Antibody | Monoclonal Antibody | dilpacimab |
| Antibody | Monoclonal Antibody | dinutuximab |
| Antibody | Monoclonal Antibody | dinutuximab beta |
| Antibody | Monoclonal Antibody | diridavumab |
| Antibody | Monoclonal Antibody | DKN-01 |
| Antibody | Monoclonal Antibody Conjugated | DNP-001 |
| Antibody | Monoclonal Antibody | DNP-002 |
| Antibody | Monoclonal Antibody | domagrozumab |
| Antibody | Monoclonal Antibody | donanemab |
| Antibody | Monoclonal Antibody | dostarlimab |
| Antibody | Monoclonal Antibody Conjugated | DP-303c |
| Antibody | Monoclonal Antibody Conjugated | DS-1062 |
| Antibody | Monoclonal Antibody Conjugated | DS-7300 |
| Antibody | Monoclonal Antibody | DS-8273 |
| Antibody | Monoclonal Antibody | dupilumab |
| Antibody | Monoclonal Antibody | durvalumab |
| Antibody | Monoclonal Antibody | durvalumab + monalizumab |
| Antibody | Monoclonal Antibody | durvalumab + oleclumab |
| Antibody | Monoclonal Antibody; Small Molecule | durvalumab + selumetinib |
| | | sulfate |
| Antibody | Monoclonal Antibody | durvalumab + tremelimumab |
| Antibody | Monoclonal Antibody | EBI-031 |
| Antibody | Monoclonal Antibody | eculizumab |
| Antibody | Monoclonal Antibody | eculizumab biosimilar |
| Antibody | Monoclonal Antibody | edrecolomab |
| Antibody | Monoclonal Antibody | efalizumab |
| Antibody | Monoclonal Antibody | efgartigimod alfa |
| Antibody | Monoclonal Antibody | efungumab |
| Antibody | Monoclonal Antibody | elezanumab |
| Antibody | Monoclonal Antibody | elgemtumab |
| Antibody | Monoclonal Antibody | elipovimab |
| Antibody | Monoclonal Antibody | elotuzumab |
| Antibody | Monoclonal Antibody | emactuzumab |
| Antibody | Monoclonal Antibody | emapalumab |
| Antibody | Bispecific Monoclonal Antibody | emicizumab |
| Antibody | Monoclonal Antibody | enamptcumab |
| Antibody | Monoclonal Antibody Conjugated | enapotamab vedotin |
| Antibody | Monoclonal Antibody Conjugated | enfortumab vedotin |
| Antibody | Monoclonal Antibody | enoblituzumab |
| Antibody | Monoclonal Antibody | ensituximab |
| Antibody | Bispecific Monoclonal Antibody | epcoritamab |
| Antibody | Monoclonal Antibody | epratuzumab |
| Antibody | Monoclonal Antibody | eptinezumab |
| Antibody | Monoclonal Antibody | erenumab |
| Antibody | Bispecific Monoclonal Antibody | ertumaxomab |
| Antibody | Bispecific Monoclonal Antibody | ERY-974 |
| Antibody | Monoclonal Antibody | etaracizumab |
| Antibody | Monoclonal Antibody | etigilimab |
| Antibody | Monoclonal Antibody | etokimab |
| Antibody | Monoclonal Antibody | etrolizumab |
| Antibody | Monoclonal Antibody | evinacumab |
| Antibody | Monoclonal Antibody | evolocumab |
| Antibody | Monoclonal Antibody; Synthetic Peptide | exenatide + ND-017 |
| Antibody | Monoclonal Antibody | F-598 |
| Antibody | Bispecific Monoclonal Antibody | faricimab |
| Antibody | Monoclonal Antibody | farletuzumab |
| Antibody | Monoclonal Antibody | fasinumab |
| Antibody | Monoclonal Antibody | FAZ-053 |
| Antibody | Monoclonal Antibody | FB-704A |
| Antibody | Monoclonal Antibody | FB-825 |
| Antibody | Antibody | FBF-001 |
| Antibody | Antibody | Ferritarg |
| Antibody | Monoclonal Antibody Conjugated | FF-21101 |
| Antibody | Monoclonal Antibody | ficlatuzumab |
| Antibody | Bispecific Monoclonal Antibody | flotetuzumab |
| Antibody | Monoclonal Antibody | FLYSYN |
| Antibody | Monoclonal Antibody | FM-101 |
| Antibody | Monoclonal Antibody Conjugated | FOR-46 |
| Antibody | Monoclonal Antibody | foralumab |
| Antibody | Monoclonal Antibody | FR-104 |
| Antibody | Monoclonal Antibody | fremanezumab |
| Antibody | Monoclonal Antibody | fresolimumab |
| Antibody | Monoclonal Antibody | FS-102 |
| Antibody | Bispecific Monoclonal Antibody | FS-118 |
| Antibody | Monoclonal Antibody | fulranumab |
| Antibody | Monoclonal Antibody | galcanezumab |
| Antibody | Monoclonal Antibody | ganitumab |
| Antibody | Monoclonal Antibody | gantenerumab |
| Antibody | Monoclonal Antibody | garadacimab |
| Antibody | Monoclonal Antibody | garetosmab |
| Antibody | Monoclonal Antibody | gatipotuzumab |
| Antibody | Monoclonal Antibody | GC-1118A |
| Antibody | Monoclonal Antibody | GEM-103 |
| Antibody | Bispecific Monoclonal Antibody | GEM-333 |
| Antibody | Bispecific Monoclonal Antibody | GEM-3PSCA |
| Antibody | Monoclonal Antibody Conjugated | gemtuzumab ozogamicin |
| Antibody | Bispecific Monoclonal Antibody | GEN-1046 |
| Antibody | Monoclonal Antibody | gevokizumab |
| Antibody | Monoclonal Antibody | gimsilumab |
| Antibody | Monoclonal Antibody | girentuximab |
| Antibody | Monoclonal Antibody Conjugated | glembatumumab vedotin |
| Antibody | Monoclonal Antibody | GLS-010 |
| Antibody | Monoclonal Antibody | GMA-102 |
| Antibody | Monoclonal Antibody | GMA-161 |
| Antibody | Monoclonal Antibody | GMA-301 |
| Antibody | Monoclonal Antibody | golimumab |
| Antibody | Monoclonal Antibody | gosuranemab |
| Antibody | Monoclonal Antibody | GR-1501 |
| Antibody | Bispecific Monoclonal Antibody | gremubamab |
| Antibody | Bispecific Monoclonal Antibody | GS-1423 |
| Antibody | Monoclonal Antibody | GSK-1070806 |
| Antibody | Monoclonal Antibody | GSK-2330811 |
| Antibody | Monoclonal Antibody | GSK-2831781 |
| Antibody | Monoclonal Antibody | GSK-3050002 |
| Antibody | Monoclonal Antibody | GSK-3174998 |
| Antibody | Monoclonal Antibody | GSK-3359609 |
| Antibody | Monoclonal Antibody | GSK-3511294 |
| Antibody | Monoclonal Antibody | GT-103 |
| Antibody | Monoclonal Antibody | guselkumab |
| Antibody | Monoclonal Antibody | GWN-323 |
| Antibody | Monoclonal Antibody | H-11 |
| Antibody | Monoclonal Antibody | HAB-21 |
| Antibody | Monoclonal Antibody | HBM-4003 |
| Antibody | Monoclonal Antibody | HDIT-101 |
| Antibody | Antibody | hepatitis B immune globulin |
| | | (human) |
| Antibody | Antibody | hepatitis C virus immune |
| | | globulin (human) |
| Antibody | Monoclonal Antibody | HLX-06 |
| Antibody | Monoclonal Antibody | HLX-07 |
| Antibody | Monoclonal Antibody | HLX-10 |
| Antibody | Monoclonal Antibody | HLX-20 |
| Antibody | Monoclonal Antibody | HPN-217 |
| Antibody | Monoclonal Antibody | HPN-424 |
| Antibody | Monoclonal Antibody | HPN-536 |
| Antibody | Monoclonal Antibody | HS-006 |
| Antibody | Monoclonal Antibody Conjugated | HTI-1066 |
| Antibody | Monoclonal Antibody | Hu8F4 |
| Antibody | Antibody | human immunoglobulin |
| | | antistaphylococcal |
| Antibody | Monoclonal Antibody | ianalumab |
| Antibody | Monoclonal Antibody | ibalizumab |
| Antibody | Monoclonal Antibody | IBI-101 |
| Antibody | Monoclonal Antibody | IBI-188 |
| Antibody | Monoclonal Antibody | IBI-306 |
| Antibody | Bispecific Monoclonal Antibody | IBI-322 |
| Antibody | Monoclonal Antibody Conjugated | ibritumomab tiuxetan |
| Antibody | Monoclonal Antibody | IC-14 |
| Antibody | Monoclonal Antibody | ICT-01 |
| Antibody | Monoclonal Antibody | idarucizumab |
| Antibody | Monoclonal Antibody | ieramilimab |
| Antibody | Monoclonal Antibody | ifabotuzumab |
| Antibody | Monoclonal Antibody | IFX-1 |
| Antibody | Monoclonal Antibody | IGEM-F |
| Antibody | Bispecific Monoclonal Antibody | IGM-2323 |
| Antibody | Antibody | immune globulin (human) |
| Antibody | Antibody | immune globulin (human) 2 |
| Antibody | Bispecific Monoclonal Antibody | INBRX-105 |
| Antibody | Monoclonal Antibody | INCAGN-1876 |
| Antibody | Monoclonal Antibody | INCAGN-1949 |
| Antibody | Monoclonal Antibody | INCAGN-2385 |
| Antibody | Monoclonal Antibody | inclacumab |
| Antibody | Monoclonal Antibody Conjugated | indatuximab ravtansine |
| Antibody | Monoclonal Antibody Conjugated | indusatumab vedotin |
| Antibody | Monoclonal Antibody | inebilizumab |
| Antibody | Monoclonal Antibody | infliximab |
| Antibody | Monoclonal Antibody | infliximab biobetter |
| Antibody | Monoclonal Antibody | infliximab biosimilar |
| Antibody | Monoclonal Antibody | INM-004 |
| Antibody | Monoclonal Antibody | inolimomab |
| Antibody | Monoclonal Antibody Conjugated | inotuzumab ozogamicin |
| Antibody | Monoclonal Antibody Conjugated | Iodine-131-Kab201 |
| Antibody | Monoclonal Antibody Conjugated | Iomab-B |
| Antibody | Monoclonal Antibody | IPH-5401 |
| Antibody | Monoclonal Antibody | ipilimumab |
| Antibody | Monoclonal Antibody | ipilimumab + nivolumab |
| Antibody | Monoclonal Antibody | isatuximab |
| Antibody | Bispecific Monoclonal Antibody | ISB-1302 |
| Antibody | Bispecific Monoclonal Antibody | ISB-1342 |
| Antibody | Monoclonal Antibody | ISB-830 |
| Antibody | Monoclonal Antibody | iscalimab |
| Antibody | Monoclonal Antibody | ISU-104 |
| Antibody | Monoclonal Antibody | itolizumab |
| Antibody | Monoclonal Antibody | ixekizumab |
| Antibody | Monoclonal Antibody | IXTM-200 |
| Antibody | Monoclonal Antibody | JMT-103 |
| Antibody | Monoclonal Antibody | JNJ-0839 |
| Antibody | Monoclonal Antibody | JNJ-3657 |
| Antibody | Monoclonal Antibody | JNJ-4500 |
| Antibody | Bispecific Monoclonal Antibody | JNJ-6372 |
| Antibody | Bispecific Monoclonal Antibody | JNJ-67571244 |
| Antibody | Bispecific Monoclonal Antibody | JNJ-7564 |
| Antibody | Bispecific Monoclonal Antibody | JNJ-7957 |
| Antibody | Bispecific Monoclonal Antibody | JNJ-9178 |
| Antibody | Monoclonal Antibody | JS-004 |
| Antibody | Monoclonal Antibody | JTX-4014 |
| Antibody | Monoclonal Antibody | JY-025 |
| Antibody | Monoclonal Antibody | K-170 |
| Antibody | Monoclonal Antibody | KHK-2823 |
| Antibody | Monoclonal Antibody | KHK-4083 |
| Antibody | Monoclonal Antibody | KHK-6640 |
| Antibody | Monoclonal Antibody Conjugated | Kid EDV |
| Antibody | Monoclonal Antibody | KLA-167 |
| Antibody | Bispecific Monoclonal Antibody | KN-026 |
| Antibody | Bispecific Monoclonal Antibody | KN-046 |
| Antibody | Monoclonal Antibody | KSI-301 |
| Antibody | Monoclonal Antibody | KY-1005 |
| Antibody | Monoclonal Antibody Conjugated | labetuzumab govitecan |
| Antibody | Monoclonal Antibody | lacnotuzumab |
| Antibody | Monoclonal Antibody | lacutamab |
| Antibody | Monoclonal Antibody Conjugated | ladiratuzumab vedotin |
| Antibody | Monoclonal Antibody | lanadelumab |
| Antibody | Monoclonal Antibody | LBL-007 |
| Antibody | Monoclonal Antibody Conjugated | LDOS-47 |
| Antibody | Monoclonal Antibody | lebrikizumab |
| Antibody | Monoclonal Antibody | lecanemab |
| Antibody | Monoclonal Antibody | Lemtrada |
| Antibody | Monoclonal Antibody | lenvervimab |
| Antibody | Monoclonal Antibody | lenzilumab |
| Antibody | Monoclonal Antibody | leronlimab |
| Antibody | Monoclonal Antibody | letolizumab |
| Antibody | Monoclonal Antibody | ligelizumab |
| Antibody | Monoclonal Antibody | lintuzumab |
| Antibody | Monoclonal Antibody; Recombinant | liraglutide + NN-8828 |
| | Peptide | |
| Antibody | Monoclonal Antibody | lirilumab |
| Antibody | Monoclonal Antibody | LKA-651 |
| Antibody | Monoclonal Antibody | LLG-783 |
| Antibody | Monoclonal Antibody | lodapolimab |
| Antibody | Monoclonal Antibody Conjugated | loncastuximab tesirine |
| Antibody | Monoclonal Antibody Conjugated | lorvotuzumab mertansine |
| Antibody | Monoclonal Antibody | LuAF-82422 |
| Antibody | Monoclonal Antibody | LuAF-87908 |
| Antibody | Monoclonal Antibody | lulizumab pegol |
| Antibody | Monoclonal Antibody | lumiliximab |
| Antibody | Monoclonal Antibody | LVGN-6051 |
| Antibody | Monoclonal Antibody | LY-3022855 |
| Antibody | Monoclonal Antibody | LY-3041658 |
| Antibody | Monoclonal Antibody | LY-3127804 |
| Antibody | Bispecific Monoclonal Antibody | LY-3434172 |
| Antibody | Antibody | LY-3435151 |
| Antibody | Antibody | LY-3454738 |
| Antibody | Monoclonal Antibody | LZM-009 |
| Antibody | Bispecific Monoclonal Antibody | M-1095 |
| Antibody | Antibody | M-254 |
| Antibody | Monoclonal Antibody | M-6495 |
| Antibody | Bispecific Monoclonal Antibody | M-802 |
| Antibody | Monoclonal Antibody | mAb-114 |
| Antibody | Monoclonal Antibody | magrolimab |
| Antibody | Monoclonal Antibody | margetuximab |
| Antibody | Monoclonal Antibody | marstacimab |
| Antibody | Monoclonal Antibody | MAU-868 |
| Antibody | Monoclonal Antibody | mavrilimumab |
| Antibody | Bispecific Monoclonal Antibody | MCLA-117 |
| Antibody | Bispecific Monoclonal Antibody | MCLA-145 |
| Antibody | Bispecific Monoclonal Antibody | MCLA-158 |
| Antibody | Monoclonal Antibody | MDX-1097 |
| Antibody | Monoclonal Antibody | MEDI-0618 |
| Antibody | Monoclonal Antibody | MEDI-1341 |
| Antibody | Monoclonal Antibody | MEDI-1814 |
| Antibody | Monoclonal Antibody | MEDI-3506 |
| Antibody | Monoclonal Antibody | MEDI-3617 + tremelimumab |
| Antibody | Monoclonal Antibody | MEDI-5117 |
| Antibody | Monoclonal Antibody Conjugated | MEDI-547 |
| Antibody | Monoclonal Antibody | MEDI-570 |
| Antibody | Bispecific Monoclonal Antibody | MEDI-5752 |
| Antibody | Bispecific Monoclonal Antibody | MEDI-7352 |
| Antibody | Monoclonal Antibody | melrilimab |
| Antibody | Monoclonal Antibody | MEN-1112 |
| Antibody | Monoclonal Antibody | mepolizumab |
| Antibody | Monoclonal Antibody | metelimumab |
| Antibody | Monoclonal Antibody | MG-1113A |
| Antibody | Monoclonal Antibody | MGA-012 |
| Antibody | Monoclonal Antibody | MGB-453 |
| Antibody | Monoclonal Antibody Conjugated | MGC-018 |
| Antibody | Bispecific Monoclonal Antibody | MGD-013 |
| Antibody | Monoclonal Antibody | MIL-62 |
| Antibody | Monoclonal Antibody | milatuzumab |
| Antibody | Monoclonal Antibody | mirikizumab |
| Antibody | Monoclonal Antibody Conjugated | mirvetuximab soravtansine |
| Antibody | Monoclonal Antibody | mitazalimab |
| Antibody | Monoclonal Antibody | MK-1308 |
| Antibody | Monoclonal Antibody | MK-1654 |
| Antibody | Monoclonal Antibody | MK-3655 |
| Antibody | Monoclonal Antibody | MK-4166 |
| Antibody | Monoclonal Antibody | MK-4280 |
| Antibody | Monoclonal Antibody | MK-5890 |
| Antibody | Monoclonal Antibody | mogamulizumab |
| Antibody | Monoclonal Antibody | monalizumab |
| Antibody | Monoclonal Antibody Conjugated | Monoclonal Antibody |
| | | Conjugate to Target CD20 |
| | | for Leukemia and Burkitt |
| | | Lymphoma |
| Antibody | Monoclonal Antibody Conjugated | Monoclonal Antibody |
| | | Conjugate to Target CD45 |
| | | for Oncology |
| Antibody | Monoclonal Antibody Conjugated | Monoclonal Antibody |
| | | Conjugate to Target CEA for |
| | | Metastatic Liver, Colorectal |
| | | Cancer and Solid Tumor |
| Antibody | Monoclonal Antibody Conjugated | Monoclonal Antibody |
| | | Conjugate to Target |
| | | CEACAM5 for Non Small |
| | | Cell Lung Cancer and |
| | | Metastatic Colorectal Cancer |
| Antibody | Monoclonal Antibody Conjugated | Monoclonal Antibody |
| | | Conjugated to Target |
| | | EPCAM for Colorectal |
| | | Cancer |
| Antibody | Monoclonal Antibody Conjugated | Monoclonal Antibody |
| | | Conjugated to Target PSMA |
| | | for Prostate Cancer |
| Antibody | Monoclonal Antibody | Monoclonal Antibody for |
| | | Coronavirus Disease 2019 |
| | | (COVID-19) |
| Antibody | Monoclonal Antibody | Monoclonal Antibody for |
| | | Dengue |
| Antibody | Monoclonal Antibody | Monoclonal Antibody to |
| | | Antagonize IL-2R Beta for |
| | | Celiac Disease, Oncology |
| | | and Tropical Spastic |
| | | Paraparesis |
| Antibody | Monoclonal Antibody | Monoclonal Antibody to |
| | | Inhibit ANXA3 for |
| | | Hepatocellular Carcinoma |
| Antibody | Monoclonal Antibody | Monoclonal Antibody to |
| | | Inhibit CD4 for HIV-1 |
| Antibody | Monoclonal Antibody | Monoclonal Antibody to |
| | | Inhibit GD2 for Oncology |
| Antibody | Monoclonal Antibody | Monoclonal Antibody to |
| | | Inhibit Glycoprotein 120 for |
| | | HIV-1 infections |
| Antibody | Monoclonal Antibody | Monoclonal Antibody to |
| | | Inhibit IL-17A and IL-17F for |
| | | Unspecified Indication |
| Antibody | Monoclonal Antibody | Monoclonal Antibody to |
| | | Inhibit PD-L1 for Solid |
| | | Tumor |
| Antibody | Monoclonal Antibody | Monoclonal Antibody to |
| | | Inhibit PD1 for Solid Tumors |
| Antibody | Monoclonal Antibody | Monoclonal Antibody to |
| | | Inhibit TNF-Alpha for |
| | | Dupuytren's contracture |
| Antibody | Monoclonal Antibody Conjugated | Monoclonal Antibody to |
| | | Target CD66b for Blood |
| | | Cancer and Metabolic |
| | | Disorders |
| Antibody | Monoclonal Antibody | Monoclonal Antibody to |
| | | Target GP41 for HIV |
| | | Infections |
| Antibody | Monoclonal Antibody | MOR-106 |
| Antibody | Monoclonal Antibody | MOR-202 |
| Antibody | Monoclonal Antibody Conjugated | MORAb-202 |
| Antibody | Bispecific Monoclonal Antibody | mosunetuzumab |
| Antibody | Monoclonal Antibody Conjugated | moxetumomab pasudotox |
| Antibody | Monoclonal Antibody | MSB-2311 |
| Antibody | Monoclonal Antibody | MSC-1 |
| Antibody | Monoclonal Antibody | MT-2990 |
| Antibody | Monoclonal Antibody | MT-3921 |
| Antibody | Monoclonal Antibody | murlentamab |
| Antibody | Monoclonal Antibody | muromonab-CD3 |
| Antibody | Monoclonal Antibody | MVT-5873 |
| Antibody | Monoclonal Antibody | namilumab |
| Antibody | Monoclonal Antibody Conjugated | naratuximab emtansine |
| Antibody | Monoclonal Antibody | narsoplimab |
| Antibody | Monoclonal Antibody | natalizumab |
| Antibody | Monoclonal Antibody | natalizumab biosimilar |
| Antibody | Bispecific Monoclonal Antibody | navicixizumab |
| Antibody | Monoclonal Antibody | naxitamab |
| Antibody | Monoclonal Antibody | NC-318 |
| Antibody | Monoclonal Antibody | nebacumab |
| Antibody | Monoclonal Antibody | necitumumab |
| Antibody | Monoclonal Antibody | nemolizumab |
| Antibody | Monoclonal Antibody | netakimab |
| Antibody | Monoclonal Antibody | NGM-120 |
| Antibody | Monoclonal Antibody | NI-006 |
| Antibody | Monoclonal Antibody | NI-0101 |
| Antibody | Monoclonal Antibody | nidanilimab |
| Antibody | Monoclonal Antibody | nimacimab |
| Antibody | Monoclonal Antibody | nimotuzumab |
| Antibody | Monoclonal Antibody | nimotuzumab biosimilar |
| Antibody | Monoclonal Antibody | nipocalimab |
| Antibody | Monoclonal Antibody | nirsevimab |
| Antibody | Monoclonal Antibody | NIS-793 |
| Antibody | Monoclonal Antibody | nivolumab |
| Antibody | Monoclonal Antibody Conjugated | NJH-395 |
| Antibody | Bispecific Monoclonal Antibody | NNC-03653769A |
| Antibody | Antibody | NP-024 |
| Antibody | Antibody | NP-025 |
| Antibody | Monoclonal Antibody | NP-137 |
| Antibody | Monoclonal Antibody | NPC-21 |
| Antibody | Bispecific Monoclonal Antibody | NXT-007 |
| Antibody | Monoclonal Antibody | NZV-930 |
| Antibody | Monoclonal Antibody | obexelimab |
| Antibody | Monoclonal Antibody | OBI-888 |
| Antibody | Monoclonal Antibody Conjugated | OBI-999 |
| Antibody | Monoclonal Antibody | obiltoxaximab |
| Antibody | Monoclonal Antibody | obinutuzumab |
| Antibody | Monoclonal Antibody Conjugated | OBT-076 |
| Antibody | Monoclonal Antibody | ocaratuzumab |
| Antibody | Monoclonal Antibody | ocrelizumab |
| Antibody | Bispecific Monoclonal Antibody | odronextamab |
| Antibody | Monoclonal Antibody | ofatumumab |
| Antibody | Monoclonal Antibody | olaratumab |
| Antibody | Monoclonal Antibody | oleclumab |
| Antibody | Monoclonal Antibody | olendalizumab |
| Antibody | Monoclonal Antibody | olinvacimab |
| Antibody | Monoclonal Antibody | olokizumab |
| Antibody | Monoclonal Antibody | omalizumab |
| Antibody | Monoclonal Antibody | omalizumab biosimilar |
| Antibody | Monoclonal Antibody Conjugated | omburtamab |
| Antibody | Monoclonal Antibody | omodenbamab |
| Antibody | Monoclonal Antibody | ONC-392 |
| Antibody | Monoclonal Antibody | ontamalimab |
| Antibody | Monoclonal Antibody | ontuxizumab |
| Antibody | Monoclonal Antibody | opicinumab |
| Antibody | Monoclonal Antibody | oregovomab |
| Antibody | Monoclonal Antibody | orilanolimab |
| Antibody | Monoclonal Antibody | orticumab |
| Antibody | Monoclonal Antibody | OS-2966 |
| Antibody | Monoclonal Antibody | OSE-127 |
| Antibody | Monoclonal Antibody | osocimab |
| Antibody | Monoclonal Antibody | otelixizumab |
| Antibody | Monoclonal Antibody | otilimab |
| Antibody | Monoclonal Antibody | otlertuzumab |
| Antibody | Monoclonal Antibody Conjugated | OTSA-101 |
| Antibody | Monoclonal Antibody Conjugated | OXS-1750 |
| Antibody | Monoclonal Antibody Conjugated | OXS-2050 |
| Antibody | Monoclonal Antibody | ozoralizumab |
| Antibody | Monoclonal Antibody | P-2G12 |
| Antibody | Monoclonal Antibody | pagibaximab |
| Antibody | Monoclonal Antibody | palivizumab |
| Antibody | Monoclonal Antibody | pamrevlumab |
| Antibody | Monoclonal Antibody | panitumumab |
| Antibody | Monoclonal Antibody | panobacumab |
| Antibody | Bispecific Monoclonal Antibody | pasotuxizumab |
| Antibody | Monoclonal Antibody | PAT-SC1 |
| Antibody | Monoclonal Antibody | patritumab |
| Antibody | Monoclonal Antibody | PC-mAb |
| Antibody | Monoclonal Antibody | PD-0360324 |
| Antibody | Monoclonal Antibody | pembrolizumab |
| Antibody | Monoclonal Antibody | pepinemab |
| Antibody | Monoclonal Antibody | pertuzumab |
| Antibody | Monoclonal Antibody | pertuzumab + trastuzumab |
| Antibody | Monoclonal Antibody | PF-04518600 |
| Antibody | Monoclonal Antibody | PF-06480605 |
| Antibody | Antibody | PF-06730512 |
| Antibody | Monoclonal Antibody | PF-06823859 |
| Antibody | Bispecific Monoclonal Antibody | PF-06863135 |
| Antibody | Monoclonal Antibody | pidilizumab |
| Antibody | Antibody | pit viper snake [Crotalidae] |
| | | (polyvalent) immunoglobulin |
| | | F(ab′)2 antivenom [equine] |
| Antibody | Bispecific Monoclonal Antibody | plamotamab |
| Antibody | Monoclonal Antibody | PNT-001 |
| Antibody | Monoclonal Antibody Conjugated | polatuzumab vedotin |
| Antibody | Antibody | PolyCAb |
| Antibody | Monoclonal Antibody | pozelimab |
| Antibody | Monoclonal Antibody | prasinezumab |
| Antibody | Monoclonal Antibody | pritumumab |
| Antibody | Monoclonal Antibody | PRL3-ZUMAB |
| Antibody | Monoclonal Antibody | prolgolimab |
| Antibody | Monoclonal Antibody | PRV-300 |
| Antibody | Bispecific Monoclonal Antibody | PRV-3279 |
| Antibody | Monoclonal Antibody | PRX-004 |
| Antibody | Bispecific Monoclonal Antibody | PSB-205 |
| Antibody | Monoclonal Antibody | PTX-35 |
| Antibody | Monoclonal Antibody | PTZ-329 |
| Antibody | Monoclonal Antibody | PTZ-522 |
| Antibody | Monoclonal Antibody | quetmolimab |
| Antibody | Monoclonal Antibody | QX-002N |
| Antibody | Monoclonal Antibody | R-1549 |
| Antibody | Monoclonal Antibody | rabies immune globulin |
| | | (human) |
| Antibody | Monoclonal Antibody | racotumomab |
| Antibody | Monoclonal Antibody Conjugated | Radspherin |
| Antibody | Monoclonal Antibody | ramucirumab |
| Antibody | Monoclonal Antibody | ranibizumab |
| Antibody | Monoclonal Antibody | ranibizumab biosimilar |
| Antibody | Monoclonal Antibody | ranibizumab SR |
| Antibody | Monoclonal Antibody | ravagalimab |
| Antibody | Monoclonal Antibody | ravulizumab |
| Antibody | Monoclonal Antibody | ravulizumab next generation |
| Antibody | Monoclonal Antibody | raxibacumab |
| Antibody | Monoclonal Antibody Conjugated | RC-48 |
| Antibody | Monoclonal Antibody | |
| Antibody | Monoclonal Antibody | REGN-3048 |
| Antibody | Monoclonal Antibody | REGN-3051 |
| Antibody | Monoclonal Antibody | REGN-3500 |
| Antibody | Bispecific Monoclonal Antibody | REGN-4018 |
| Antibody | Monoclonal Antibody | REGN-4461 |
| Antibody | Antibody | REGN-5069 |
| Antibody | Bispecific Monoclonal Antibody | REGN-5458 |
| Antibody | Bispecific Monoclonal Antibody | REGN-5459 |
| Antibody | Bispecific Monoclonal Antibody | REGN-5678 |
| Antibody | Monoclonal Antibody | REGN-5713 |
| Antibody | Monoclonal Antibody | REGN-5714 |
| Antibody | Monoclonal Antibody | REGN-5715 |
| Antibody | Monoclonal Antibody | relatlimab |
| Antibody | Monoclonal Antibody | reslizumab |
| Antibody | Antibody | respiratory syncytial virus |
| | | immune globulin (human) |
| Antibody | Monoclonal Antibody | RG-6125 |
| Antibody | Bispecific Monoclonal Antibody | RG-6139 |
| Antibody | Monoclonal Antibody | RG-6149 |
| Antibody | Bispecific Monoclonal Antibody; | RG-6160 |
| | Monoclonal Antibody | |
| Antibody | Monoclonal Antibody | RG-6292 |
| Antibody | Antibody | RG-70240 |
| Antibody | Monoclonal Antibody Conjugated | RG-7861 |
| Antibody | Bispecific Monoclonal Antibody | RG-7992 |
| Antibody | Antibody | rho(D) immune globulin |
| | | (human) |
| Antibody | Monoclonal Antibody | rilotumumab |
| Antibody | Monoclonal Antibody | risankizumab |
| Antibody | Monoclonal Antibody | rituximab |
| Antibody | Monoclonal Antibody | rituximab biosimilar |
| Antibody | Bispecific Monoclonal Antibody | RO-7082859 |
| Antibody | Bispecific Monoclonal Antibody | RO-7121661 |
| Antibody | Monoclonal Antibody | roledumab |
| Antibody | Bispecific Monoclonal Antibody | romilkimab |
| Antibody | Monoclonal Antibody | romosozumab |
| Antibody | Monoclonal Antibody Conjugated | rovalpituzumab tesirine |
| Antibody | Monoclonal Antibody | rozanolixizumab |
| Antibody | Monoclonal Antibody Conjugated | rozibafusp alfa |
| Antibody | Monoclonal Antibody | RZ-358 |
| Antibody | Antibody | SAB-301 |
| Antibody | Monoclonal Antibody Conjugated | sacituzumab govitecan |
| Antibody | Monoclonal Antibody | SAIT-301 |
| Antibody | Monoclonal Antibody Conjugated | SAR-408701 |
| Antibody | Monoclonal Antibody | SAR-439459 |
| Antibody | Bispecific Monoclonal Antibody | SAR-440234 |
| Antibody | Monoclonal Antibody | SAR-441236 |
| Antibody | Monoclonal Antibody | sarilumab |
| Antibody | Monoclonal Antibody | sasanlimab |
| Antibody | Monoclonal Antibody | satralizumab |
| Antibody | Monoclonal Antibody Conjugated | SC-003 |
| Antibody | Antibody | scorpion (polyvalent) |
| | | immunoglobulin F(ab′)2 |
| | | antivenom |
| Antibody | Antibody | scorpion [centruroides] |
| | | (polyvalent) immunoglobulin |
| | | F(ab′) 2 antivenom [equine] |
| Antibody | Monoclonal Antibody | SCT-200 |
| Antibody | Monoclonal Antibody | SCT-630 |
| Antibody | Monoclonal Antibody | SEA-BCMA |
| Antibody | Monoclonal Antibody | SEA-CD40 |
| Antibody | Monoclonal Antibody | secukinumab |
| Antibody | Monoclonal Antibody | selicrelumab |
| Antibody | Monoclonal Antibody | semorinemab |
| Antibody | Monoclonal Antibody | setrusumab |
| Antibody | Monoclonal Antibody Conjugated | SGNCD-228A |
| Antibody | Monoclonal Antibody Conjugated | SGNCD-47M |
| Antibody | Antibody | SHR-1209 |
| Antibody | Monoclonal Antibody | SHR-1316 |
| Antibody | Monoclonal Antibody | siltuximab |
| Antibody | Monoclonal Antibody | Simponi Aria |
| Antibody | Monoclonal Antibody | sintilimab |
| Antibody | Monoclonal Antibody | siplizumab |
| Antibody | Monoclonal Antibody | sirukumab |
| Antibody | Monoclonal Antibody Conjugated | SKB-264 |
| Antibody | Monoclonal Antibody | solanezumab |
| Antibody | Monoclonal Antibody | spartalizumab |
| Antibody | Monoclonal Antibody | spesolimab |
| Antibody | Monoclonal Antibody | SRF-617 |
| Antibody | Monoclonal Antibody | SSS-07 |
| Antibody | Monoclonal Antibody | STIA-1014 |
| Antibody | Monoclonal Antibody Conjugated | STRO-001 |
| Antibody | Monoclonal Antibody Conjugated | STRO-002 |
| Antibody | Monoclonal Antibody | Sulituzumab |
| Antibody | Monoclonal Antibody | sutimlimab |
| Antibody | Monoclonal Antibody | suvratoxumab |
| Antibody | Monoclonal Antibody Conjugated | SYD-1875 |
| Antibody | Monoclonal Antibody | Sym-015 |
| Antibody | Monoclonal Antibody | Sym-021 |
| Antibody | Monoclonal Antibody | Sym-022 |
| Antibody | Monoclonal Antibody | Sym-023 |
| Antibody | Monoclonal Antibody | SYN-004 |
| Antibody | Monoclonal Antibody | SYN-023 |
| Antibody | Monoclonal Antibody | TAB-014 |
| Antibody | Monoclonal Antibody | TAB-08 |
| Antibody | Monoclonal Antibody | tafasitamab |
| Antibody | Antibody | taipan [Oxyuranus |
| | | scutellatus] antivenom |
| | | [equine] |
| Antibody | Monoclonal Antibody | TAK-079 |
| Antibody | Monoclonal Antibody Conjugated | TAK-164 |
| Antibody | Monoclonal Antibody | talacotuzumab |
| Antibody | Monoclonal Antibody | tanezumab |
| Antibody | Monoclonal Antibody Conjugated | telisotuzumab vedotin |
| Antibody | Monoclonal Antibody | temelimab |
| Antibody | Monoclonal Antibody | teplizumab |
| Antibody | Monoclonal Antibody | teprotumumab |
| Antibody | Monoclonal Antibody | tesidolumab |
| Antibody | Antibody | tetanus immune globulin |
| Antibody | Monoclonal Antibody | tezepelumab |
| Antibody | Monoclonal Antibody Conjugated | TF-2 |
| Antibody | Bispecific Monoclonal Antibody | TG-1801 |
| Antibody | Monoclonal Antibody | THR-317 |
| Antibody | Bispecific Monoclonal Antibody | tibulizumab |
| Antibody | Monoclonal Antibody | tilavonemab |
| Antibody | Monoclonal Antibody | tildrakizumab |
| Antibody | Monoclonal Antibody | timigutuzumab |
| Antibody | Monoclonal Antibody | timolumab |
| Antibody | Monoclonal Antibody | tiragolumab |
| Antibody | Monoclonal Antibody | tislelizumab |
| Antibody | Monoclonal Antibody Conjugated | tisotumab vedotin |
| Antibody | Monoclonal Antibody | TJC-4 |
| Antibody | Monoclonal Antibody | TJD-5 |
| Antibody | Monoclonal Antibody | TJM-2 |
| Antibody | Monoclonal Antibody | TM-123 |
| Antibody | Bispecific Monoclonal Antibody | TMB-365 |
| Antibody | Bispecific Monoclonal Antibody | TNB-383B |
| Antibody | Monoclonal Antibody | tocilizumab |
| Antibody | Monoclonal Antibody | tocilizumab biosimilar |
| Antibody | Monoclonal Antibody | tomaralimab |
| Antibody | Monoclonal Antibody | tomuzotuximab |
| Antibody | Monoclonal Antibody | toripalimab |
| Antibody | Monoclonal Antibody | tosatoxumab |
| Antibody | Monoclonal Antibody Conjugated | tositumomab + Iodine I 131 |
| | | tositumomab |
| Antibody | Monoclonal Antibody | tralokinumab |
| Antibody | Monoclonal Antibody | trastuzumab |
| Antibody | Monoclonal Antibody | trastuzumab biosimilar |
| Antibody | Monoclonal Antibody Conjugated | trastuzumab deruxtecan |
| Antibody | Monoclonal Antibody Conjugated | trastuzumab duocarmazine |
| Antibody | Monoclonal Antibody Conjugated | trastuzumab emtansine |
| Antibody | Monoclonal Antibody | tremelimumab |
| Antibody | Monoclonal Antibody | trevogrumab |
| Antibody | Monoclonal Antibody | TRK-950 |
| Antibody | Monoclonal Antibody Conjugated | TRPH-222 |
| Antibody | Monoclonal Antibody | TTX-030 |
| Antibody | Monoclonal Antibody Conjugated | TX-250 |
| Antibody | Monoclonal Antibody Conjugated | U-31402 |
| Antibody | Monoclonal Antibody | U-31784 |
| Antibody | Monoclonal Antibody | UB-221 |
| Antibody | Monoclonal Antibody | UB-421 |
| Antibody | Monoclonal Antibody | UB-621 |
| Antibody | Monoclonal Antibody | ublituximab |
| Antibody | Monoclonal Antibody; Small Molecule | ublituximab + umbralisib |
| | | tosylate |
| Antibody | Monoclonal Antibody | UBP-1213 |
| Antibody | Monoclonal Antibody | UC-961 |
| Antibody | Monoclonal Antibody | UCB-0107 |
| Antibody | Monoclonal Antibody | UCB-6114 |
| Antibody | Monoclonal Antibody | UCB-7858 |
| Antibody | Monoclonal Antibody | ulocuplumab |
| Antibody | Monoclonal Antibody | urelumab |
| Antibody | Monoclonal Antibody | ustekinumab |
| Antibody | Monoclonal Antibody | ustekinumab biosimilar |
| Antibody | Monoclonal Antibody | utomilumab |
| Antibody | Monoclonal Antibody Conjugated | vadastuximab talirine |
| Antibody | Bispecific Monoclonal Antibody | vanucizumab |
| Antibody | Antibody | |
| Antibody | Monoclonal Antibody | varisacumab |
| Antibody | Monoclonal Antibody | varlilumab |
| Antibody | Monoclonal Antibody | vedolizumab |
| Antibody | Monoclonal Antibody | veltuzumab |
| Antibody | Monoclonal Antibody | VIR-2482 |
| Antibody | Monoclonal Antibody | VIS-410 |
| Antibody | Monoclonal Antibody | VIS-649 |
| Antibody | Monoclonal Antibody | vixarelimab |
| Antibody | Monoclonal Antibody Conjugated | VLS-101 |
| Antibody | Monoclonal Antibody | vobarilizumab |
| Antibody | Monoclonal Antibody | vofatamab |
| Antibody | Monoclonal Antibody | volagidemab |
| Antibody | Monoclonal Antibody | vopratelimab |
| Antibody | Monoclonal Antibody | VRC-01 |
| Antibody | Monoclonal Antibody | VRC-07523LS |
| Antibody | Monoclonal Antibody | vunakizumab |
| Antibody | Monoclonal Antibody Conjugated | W-0101 |
| Antibody | Monoclonal Antibody | WBP-297 |
| Antibody | Antibody | |
| Antibody | Antibody | Xembify |
| Antibody | Monoclonal Antibody | xentuzumab |
| Antibody | Monoclonal Antibody | Xgeva |
| Antibody | Bispecific Monoclonal Antibody | XmAb-14045 |
| Antibody | Bispecific Monoclonal Antibody | XmAb-22841 |
| Antibody | Bispecific Monoclonal Antibody | XmAb-23104 |
| Antibody | Monoclonal Antibody Conjugated | XMT-1536 |
| Antibody | Monoclonal Antibody | XOMA-213 |
| Antibody | Monoclonal Antibody | YS-110 |
| Antibody | Monoclonal Antibody | YYB-101 |
| Antibody | Monoclonal Antibody | zagotenemab |
| Antibody | Monoclonal Antibody | zalifrelimab |
| Antibody | Monoclonal Antibody | zanolimumab |
| Antibody | Bispecific Monoclonal Antibody | zenocutuzumab |
| Antibody | Monoclonal Antibody | zolbetuximab |
| Antibody | Bispecific Monoclonal Antibody | ZW-25 |
| Antibody/Enzyme | Antibody; Recombinant Enzyme | hyaluronidase (recombinant, |
| | | human) + immune globulin |
| | | (human) |
| Antibody/protein | Fusion Protein; Monoclonal Antibody | durvalumab + oportuzumab |
| | | monatox |

TABLE 2
Peptides

| Broad | | |
|---|---|---|
| class | Molecule Type | Drug Name |
| Peptide | Synthetic Peptide | A-10 + AS-21 |
| Peptide | Synthetic Peptide | A-6 |
| Peptide | Recombinant Peptide | AB-101 |
| Peptide | Recombinant Peptide | AB-102 |
| Peptide | Recombinant Peptide | AB-301 |
| Peptide | Synthetic Peptide | abaloparatide |
| Peptide | Synthetic Peptide | abarelix |
| Peptide | Synthetic Peptide | ABT-510 |
| Peptide | Recombinant Peptide | AC-2592 |
| Peptide | Synthetic Peptide | ACP-003 |
| Peptide | Synthetic Peptide | ACP-004 |
| Peptide | Synthetic Peptide | ACP-015 |
| Peptide | Synthetic Peptide | AcPepA |
| Peptide | Synthetic Peptide | ACX-107 |
| Peptide | Synthetic Peptide | Adipotide |
| Peptide | Recombinant Peptide | ADV-P2 |
| Peptide | Synthetic Peptide | AE-3763 |
| Peptide | Synthetic Peptide | AEM-28 |
| Peptide | Synthetic Peptide | afamelanotide acetate |
| Peptide | Synthetic Peptide | AFPep |
| Peptide | Synthetic Peptide | AGM-310 |
| Peptide | Recombinant Peptide | AI-401 |
| Peptide | Synthetic Peptide | AIM-102 |
| Peptide | Recombinant Peptide | AIM-DX |
| Peptide | Synthetic Peptide | AKL-0707 |
| Peptide | Recombinant Peptide | AKS-178 |
| Peptide | Synthetic Peptide | AL-242A1 |
| Peptide | Synthetic Peptide | AL-41A1 |
| Peptide | Synthetic Peptide | AL-78898A |
| Peptide | Synthetic Peptide | albenatide |
| Peptide | Synthetic Peptide | albuvirtide LAR |
| Peptide | Synthetic Peptide | alisporivir |
| Peptide | Synthetic Peptide | ALM-201 |
| Peptide | Synthetic Peptide | Alpha-1H |
| Peptide | Synthetic Peptide | Alpha-HGA |
| Peptide | Synthetic Peptide | ALRev-1 |
| Peptide | Synthetic Peptide | ALRN-5281 |
| Peptide | Synthetic Peptide | ALRN-6924 |
| Peptide | Synthetic Peptide | ALY-688 |
| Peptide | Synthetic Peptide | AMC-303 |
| Peptide | Synthetic Peptide | Ampion |
| Peptide | Synthetic Peptide | AMY-106 |
| Peptide | Synthetic Peptide | anaritide acetate |
| Peptide | Synthetic Peptide | angiotensin II acetate |
| Peptide | Recombinant Peptide | ANX-042 |
| Peptide | Synthetic Peptide | AP-138 |
| Peptide | Recombinant Peptide | APH-0907 |
| Peptide | Synthetic Peptide | APL-180 |
| Peptide | Synthetic Peptide | APL-9 |
| Peptide | Synthetic Peptide | APP-018 |
| Peptide | Recombinant Peptide | AC-2592 |
| Peptide | Synthetic Peptide | ACP-003 |
| Peptide | Synthetic Peptide | ACP-004 |
| Peptide | Synthetic Peptide | ACP-015 |
| Peptide | Synthetic Peptide | AcPepA |
| Peptide | Synthetic Peptide | ACX-107 |
| Peptide | Synthetic Peptide | Adipotide |
| Peptide | Recombinant Peptide | ADV-P2 |
| Peptide | Synthetic Peptide | AE-3763 |
| Peptide | Synthetic Peptide | AEM-28 |
| Peptide | Synthetic Peptide | afamelanotide acetate |
| Peptide | Synthetic Peptide | AFPep |
| Peptide | Synthetic Peptide | AGM-310 |
| Peptide | Recombinant Peptide | AI-401 |
| Peptide | Synthetic Peptide | AIM-102 |
| Peptide | Recombinant Peptide | AIM-DX |
| Peptide | Synthetic Peptide | AKL-0707 |
| Peptide | Recombinant Peptide | AKS-178 |
| Peptide | Synthetic Peptide | AL-242A1 |
| Peptide | Synthetic Peptide | AL-41A1 |
| Peptide | Synthetic Peptide | AL-78898A |
| Peptide | Synthetic Peptide | albenatide |
| Peptide | Synthetic Peptide | albuvirtide LAR |
| Peptide | Synthetic Peptide | alisporivir |
| Peptide | Synthetic Peptide | ALM-201 |
| Peptide | Synthetic Peptide | Alpha-1H |
| Peptide | Synthetic Peptide | Alpha-HGA |
| Peptide | Synthetic Peptide | ALRev-1 |
| Peptide | Synthetic Peptide | ALRN-5281 |
| Peptide | Synthetic Peptide | ALRN-6924 |
| Peptide | Synthetic Peptide | ALY-688 |
| Peptide | Synthetic Peptide | AMC-303 |
| Peptide | Synthetic Peptide | Ampion |
| Peptide | Synthetic Peptide | AMY-106 |
| Peptide | Synthetic Peptide | anaritide acetate |
| Peptide | Synthetic Peptide | angiotensin II acetate |
| Peptide | Recombinant Peptide | ANX-042 |
| Peptide | Synthetic Peptide | AP-138 |
| Peptide | Recombinant Peptide | APH-0907 |
| Peptide | Synthetic Peptide | APL-180 |
| Peptide | Synthetic Peptide | APL-9 |
| Peptide | Synthetic Peptide | APP-018 |
| Peptide | Synthetic Peptide | apraglutide |
| Peptide | Synthetic Peptide | ARG-301 |
| Peptide | Synthetic Peptide | argipressin |
| Peptide | Synthetic Peptide | ARI-1778 |
| Peptide | Synthetic Peptide | Artpep-2 |
| Peptide | Synthetic Peptide | ASP-5006 |
| Peptide | Recombinant Peptide | AT-247 |
| Peptide | Recombinant Peptide | AT-270 |
| Peptide | Synthetic Peptide | ATN-161 |
| Peptide | Synthetic Peptide | atosiban |
| Peptide | Synthetic Peptide | atosiban acetate |
| Peptide | Synthetic Peptide | Atrigel-GHRP-1 |
| Peptide | Recombinant Peptide | ATX-101 |
| Peptide | Synthetic Peptide | AVE-3247 |
| Peptide | Synthetic Peptide | avexitide acetate |
| Peptide | Synthetic Peptide | B27-PD |
| Peptide | Synthetic Peptide | bacitracin |
| Peptide | Synthetic Peptide | barusiban |
| Peptide | Synthetic Peptide | BBI-11008 |
| Peptide | Synthetic Peptide | BBI-21007 |
| Peptide | Synthetic Peptide | BDM-E |
| Peptide | Synthetic Peptide | BI-456906 |
| Peptide | Synthetic Peptide | BI-473494 |
| Peptide | Synthetic Peptide | bicalutamide + leuprolide acetate |
| Peptide | Recombinant Peptide | BIOD-105 |
| Peptide | Recombinant Peptide | BIOD-107 |
| Peptide | Recombinant Peptide | BIOD-123 |
| Peptide | Recombinant Peptide | BIOD-125 |
| Peptide | Recombinant Peptide | BIOD-238 |
| Peptide | Recombinant Peptide | BIOD-250 |
| Peptide | Recombinant Peptide | BIOD-531 |
| Peptide | Recombinant Peptide | BIOD-Adjustable Basal |
| Peptide | Synthetic Peptide | bivalirudin |
| Peptide | Synthetic Peptide | bivalirudin trifluoroacetate |
| Peptide | Peptide; Synthetic Peptide | BL-3020 |
| Peptide | Synthetic Peptide | BMS-686117 |
| Peptide | Synthetic Peptide | BMTP-11 |
| Peptide | Synthetic Peptide | BN-005 |
| Peptide | Synthetic Peptide | BN-006 |
| Peptide | Synthetic Peptide | BN-008 |
| Peptide | Synthetic Peptide | BN-054 |
| Peptide | Synthetic Peptide | BNZ-1 |
| Peptide | Recombinant Peptide | BNZ-2 |
| Peptide | Synthetic Peptide | BPI-3016 |
| Peptide | Synthetic Peptide | BQ-123 |
| Peptide | Synthetic Peptide | bremelanotide acetate |
| Peptide | Synthetic Peptide | brimapitide |
| Peptide | Synthetic Peptide | BRM-521 |
| Peptide | Synthetic Peptide | BT-5528 |
| Peptide | Synthetic Peptide | BTI-410 |
| Peptide | Synthetic Peptide | bulevirtide |
| Peptide | Synthetic Peptide | buserelin acetate |
| Peptide | Synthetic Peptide | buserelin acetate ER |
| Peptide | Synthetic Peptide | Bynfezia |
| Peptide | Synthetic Peptide | C-16G2 |
| Peptide | Synthetic Peptide | calcitonin |
| Peptide | Recombinant Peptide | calcitonin DR |
| Peptide | Recombinant Peptide | Capsulin IR |
| Peptide | Recombinant Peptide | Capsulin OAD |
| Peptide | Recombinant Peptide | CAR Peptide |
| Peptide | Synthetic Peptide | carbetocin |
| Peptide | Recombinant Peptide | Cardeva |
| Peptide | Recombinant Peptide | carperitide |
| Peptide | Synthetic Peptide | CBLB-612 |
| Peptide | Synthetic Peptide | CBP-501 |
| Peptide | Synthetic Peptide | CBX-129801 |
| Peptide | Recombinant Peptide | celmoleukin |
| Peptide | Recombinant Peptide | cenderitide |
| Peptide | Synthetic Peptide | cetrorelix |
| Peptide | Synthetic Peptide | cetrorelix acetate |
| Peptide | Synthetic Peptide | CGX-1007 |
| Peptide | Synthetic Peptide | CGX-1160 |
| Peptide | Synthetic Peptide | cibinetide |
| Peptide | Synthetic Peptide | CIGB-300 |
| Peptide | Recombinant Peptide | CIGB-370 |
| Peptide | Synthetic Peptide | CIGB-500 |
| Peptide | Synthetic Peptide | CIGB-552 |
| Peptide | Synthetic Peptide | CIGB-814 |
| Peptide | Synthetic Peptide | cilengitide |
| Peptide | Recombinant Peptide | CJC-1525 |
| Peptide | Synthetic Peptide | CMS-024 |
| Peptide | Synthetic Peptide | CN-105 |
| Peptide | Recombinant Peptide | CobOral Insulin |
| Peptide | Synthetic Peptide | COG-1410 |
| Peptide | Recombinant Peptide | Combulin |
| Peptide | Synthetic Peptide | corticorelin acetate |
| Peptide | Synthetic Peptide | corticotropin |
| Peptide | Synthetic Peptide | cosyntropin |
| Peptide | Synthetic Peptide | cosyntropin SR |
| Peptide | Synthetic Peptide | CPT-31 |
| Peptide | Synthetic Peptide | CTCE-9908 |
| Peptide | Recombinant Peptide | DACRA-042 |
| Peptide | Recombinant Peptide | DACRA-089 |
| Peptide | Synthetic Peptide | dalazatide |
| Peptide | Synthetic Peptide | danegaptide |
| Peptide | Synthetic Peptide | dasiglucagon |
| Peptide | Synthetic Peptide | DasKloster-0274-01 |
| Peptide | Synthetic Peptide | davunetide |
| Peptide | Synthetic Peptide | DD-04107 |
| Peptide | Synthetic Peptide | degarelix acetate |
| Peptide | Synthetic Peptide | delcasertib acetate |
| Peptide | Synthetic Peptide | delmitide acetate |
| Peptide | Synthetic Peptide | Dennexin |
| Peptide | Synthetic Peptide | Des-Asp Angiotensin 1 |
| Peptide | Recombinant Peptide | desirudin |
| Peptide | Synthetic Peptide | desmopressin |
| Peptide | Synthetic Peptide | desmopressin acetate |
| Peptide | Synthetic Peptide | desmopressin acetate ODT |
| Peptide | Synthetic Peptide | DiaPep-277 |
| Peptide | Synthetic Peptide | difelikefalin |
| Peptide | Synthetic Peptide | Dipep |
| Peptide | Synthetic Peptide | disitertide |
| Peptide | Synthetic Peptide | DMI-4983 |
| Peptide | Synthetic Peptide | dolcanatide |
| Peptide | Synthetic Peptide | DP-2018 |
| Peptide | Synthetic Peptide | DPC-016 |
| Peptide | Synthetic Peptide | DT-109 |
| Peptide | Synthetic Peptide | DT-110 |
| Peptide | Synthetic Peptide | DTI-100 |
| Peptide | Synthetic Peptide | DTI-117 |
| Peptide | Synthetic Peptide | dusquetide |
| Peptide | Synthetic Peptide | Dyofins |
| Peptide | Synthetic Peptide | E-21R |
| Peptide | Synthetic Peptide | EA-230 |
| Peptide | Recombinant Peptide | EB-613 |
| Peptide | Synthetic Peptide | Edotreotide Labeled Yttrium 90 |
| Peptide | Synthetic Peptide | edotreotide lutetium Lu-177 |
| Peptide | Synthetic Peptide | edratide |
| Peptide | Recombinant Peptide | efpeglenatide |
| Peptide | Recombinant Peptide; Synthetic Peptide | efpeglenatide + HM-12470 |
| Peptide | Synthetic Peptide | elamipretide hydrochloride |
| Peptide | Synthetic Peptide | elcatonin |
| Peptide | Synthetic Peptide | ELIGO-3233 |
| Peptide | Synthetic Peptide | elsiglutide |
| Peptide | Recombinant Peptide | endostatin |
| Peptide | Synthetic Peptide | enfuvirtide |
| Peptide | Peptide; Synthetic Peptide | Engedi-1000 |
| Peptide | Synthetic Peptide | ENKASTIM-iv |
| Peptide | Synthetic Peptide | EP-100 |
| Peptide | Synthetic Peptide | EP-302 |
| Peptide | Synthetic Peptide | EP-342 |
| Peptide | Synthetic Peptide | EP-94 |
| Peptide | Synthetic Peptide | EPO-018B |
| Peptide | Synthetic Peptide | eptifibatide |
| Peptide | Recombinant Peptide | ES-135 |
| Peptide | Synthetic Peptide | etelcalcetide hydrochloride |
| Peptide | Synthetic Peptide | ETX-112 |
| Peptide | Synthetic Peptide | Evitar |
| Peptide | Synthetic Peptide | exenatide |
| Peptide | Synthetic Peptide | exenatide + Synthetic Peptide 1 |
| Peptide | Synthetic Peptide | exenatide + Synthetic Peptide 2 |
| Peptide | Synthetic Peptide | exenatide biobetter |
| Peptide | Synthetic Peptide | exenatide biosimilar |
| Peptide | Synthetic Peptide | exenatide CR |
| Peptide | Synthetic Peptide | exenatide ER |
| Peptide | Synthetic Peptide | exenatide Once Monthly |
| Peptide | Synthetic Peptide | exenatide SR |
| Peptide | Synthetic Peptide | exendin-(9-39) |
| Peptide | Synthetic Peptide | EXT-307 |
| Peptide | Synthetic Peptide | EXT-405 |
| Peptide | Synthetic Peptide | EXT-418 |
| Peptide | Synthetic Peptide | EXT-600 |
| Peptide | Synthetic Peptide | EXT-607 |
| Peptide | Synthetic Peptide | EXT-705 |
| Peptide | Recombinant Peptide | Extendin-Fc |
| Peptide | Synthetic Peptide | FE-204205 |
| Peptide | Synthetic Peptide | FF-3 |
| Peptide | Recombinant Peptide | Fiasp |
| Peptide | Synthetic Peptide | FM-19 |
| Peptide | Synthetic Peptide | FNS-007 |
| Peptide | Synthetic Peptide | forigerimod acetate |
| Peptide | Synthetic Peptide | Foxy-5 |
| Peptide | Synthetic Peptide | FP-001 |
| Peptide | Synthetic Peptide | FP-002 |
| Peptide | Synthetic Peptide | FP-005 |
| Peptide | Synthetic Peptide | FPP-003 |
| Peptide | Recombinant Peptide | FT-105 |
| Peptide | Synthetic Peptide | FX-06 |
| Peptide | Synthetic Peptide | G-3215 |
| Peptide | Synthetic Peptide | ganirelix acetate |
| Peptide | Synthetic Peptide | glatiramer acetate |
| Peptide | Synthetic Peptide | glatiramer acetate ER |
| Peptide | Synthetic Peptide | glatiramer biosimilar |
| Peptide | Synthetic Peptide | glepaglutide |
| Peptide | Recombinant Peptide | GLP-1 |
| Peptide | Recombinant Peptide | glucagon |
| Peptide | Recombinant Peptide | glucagon biosimilar |
| Peptide | Recombinant Peptide | Glucagon-Like Peptide-1 + insulin |
| | | human |
| Peptide | Synthetic Peptide | glucosaminylmuramyl dipeptide |
| Peptide | Synthetic Peptide | GM-6 |
| Peptide | Synthetic Peptide | GO-2032c |
| Peptide | Synthetic Peptide | golotimod |
| Peptide | Synthetic Peptide | gonadorelin |
| Peptide | Synthetic Peptide | gonadorelin acetate |
| Peptide | Synthetic Peptide | goserelin |
| Peptide | Synthetic Peptide | goserelin acetate |
| Peptide | Synthetic Peptide | goserelin ER |
| Peptide | Synthetic Peptide | goserelin LA |
| Peptide | Synthetic Peptide | goserelin SR |
| Peptide | Recombinant Peptide | GP-40031 |
| Peptide | Synthetic Peptide | GSAO |
| Peptide | Synthetic Peptide | HaemoPlax |
| Peptide | Synthetic Peptide | hbEGF |
| Peptide | Recombinant Peptide | HDV-I |
| Peptide | Synthetic Peptide | hepcidin acetate |
| Peptide | Synthetic Peptide | histrelin |
| Peptide | Recombinant Peptide | HM-12460A |
| Peptide | Recombinant Peptide | HM-12470 |
| Peptide | Recombinant Peptide | HM-12480 |
| Peptide | Recombinant Peptide | HM-15136 |
| Peptide | Synthetic Peptide | HM-15211 |
| Peptide | Synthetic Peptide | Homspera |
| Peptide | Synthetic Peptide | HPI-1201 |
| Peptide | Synthetic Peptide | HPI-201 |
| Peptide | Synthetic Peptide | HPI-363 |
| Peptide | Synthetic Peptide | hPTH-137 |
| Peptide | Synthetic Peptide | HTD-4010 |
| Peptide | Synthetic Peptide | HTL-001 |
| Peptide | Recombinant Peptide | Humalog |
| Peptide | Synthetic Peptide | HXTC-901 |
| Peptide | Synthetic Peptide | Hydrogel Exenatide |
| Peptide | Synthetic Peptide | icatibant acetate |
| Peptide | Synthetic Peptide | IIIM-1 |
| Peptide | Synthetic Peptide | IMB-1007 |
| Peptide | Synthetic Peptide | ImmTher |
| Peptide | Recombinant Peptide | insulin |
| Peptide | Recombinant Peptide | insulin (bovine) |
| Peptide | Recombinant Peptide | insulin aspart |
| Peptide | Recombinant Peptide | insulin aspart 1 |
| Peptide | Recombinant Peptide | insulin aspart biosimilar |
| Peptide | Recombinant Peptide | insulin aspart injection |
| Peptide | Recombinant Peptide | insulin degludec |
| Peptide | Recombinant Peptide | insulin degludec LAR |
| Peptide | Recombinant Peptide | insulin detemir |
| Peptide | Recombinant Peptide | insulin glargine |
| Peptide | Recombinant Peptide | insulin glargine 1 |
| Peptide | Recombinant Peptide | insulin glargine biosimilar |
| Peptide | Recombinant Peptide | insulin glargine biosimilar 2 |
| Peptide | Recombinant Peptide | insulin glargine ER |
| Peptide | Recombinant Peptide | insulin glargine LA |
| Peptide | Recombinant Peptide | insulin glulisine |
| Peptide | Recombinant Peptide | insulin human |
| Peptide | Recombinant Peptide | insulin human (recombinant) |
| Peptide | Recombinant Peptide | insulin human 1 |
| Peptide | Recombinant Peptide | Insulin Human 30/70 Mix Marvel |
| Peptide | Recombinant Peptide | Insulin Human Long Marvel |
| Peptide | Recombinant Peptide | Insulin Human Rapid Marvel |
| Peptide | Recombinant Peptide | insulin human U100 |
| Peptide | Recombinant Peptide | insulin human zinc |
| Peptide | Recombinant Peptide | insulin I 131 |
| Peptide | Recombinant Peptide | insulin isophane |
| Peptide | Recombinant Peptide | insulin isophane human |
| Peptide | Recombinant Peptide | insulin lispro |
| Peptide | Recombinant Peptide | insulin lispro 2 |
| Peptide | Recombinant Peptide | insulin lispro U100 |
| Peptide | Recombinant Peptide | insulin lispro U200 |
| Peptide | Recombinant Peptide | insulin lispro U300 |
| Peptide | Recombinant Peptide | insulin neutral |
| Peptide | Recombinant Peptide | insulin peglispro |
| Peptide | Recombinant Peptide | insulin tregopil |
| Peptide | Recombinant Peptide | Insulin-PH20 |
| Peptide | Recombinant Peptide | Insulin-B12 Conjugate |
| Peptide | Recombinant Peptide | insulin, neutral |
| Peptide | Recombinant Peptide | Insuman |
| Peptide | Synthetic Peptide | IP-1510 |
| Peptide | Synthetic Peptide | IP-1510D |
| Peptide | Synthetic Peptide | ipamorelin |
| Peptide | Synthetic Peptide | IPL-344 |
| Peptide | Synthetic Peptide | IPP-102199 |
| Peptide | Synthetic Peptide | IPP-204106 |
| Peptide | Recombinant Peptide | Ir-CPI |
| Peptide | Synthetic Peptide | ISF-402 |
| Peptide | Recombinant Peptide | isophane protamine recombinant |
| | | human insulin |
| Peptide | Synthetic Peptide | ITCA-650 |
| Peptide | Synthetic Peptide | ITF-1697 |
| Peptide | Recombinant Peptide | ITF-2984 |
| Peptide | Recombinant Peptide | JDSCR-103 |
| Peptide | Synthetic Peptide | JMR-132 |
| Peptide | Synthetic Peptide | JNJ-26366821 |
| Peptide | Synthetic Peptide | JNJ-38488502 |
| Peptide | Synthetic Peptide | K-13 |
| Peptide | Synthetic Peptide | kahalalide F |
| Peptide | Synthetic Peptide | KAI-1678 |
| Peptide | Recombinant Peptide | KBP-088 |
| Peptide | Synthetic Peptide | KES-0001 |
| Peptide | Synthetic Peptide | Kisspeptin-10 |
| Peptide | Synthetic Peptide | KRX-0402 |
| Peptide | Synthetic Peptide | KSL-W |
| Peptide | Recombinant Peptide | KUR-112 |
| Peptide | Recombinant Peptide | KUR-113 |
| Peptide | Synthetic Peptide | L-1AD3 |
| Peptide | Recombinant Peptide | LAI-287 |
| Peptide | Recombinant Peptide | LAI-338 |
| Peptide | Synthetic Peptide | lanreotide acetate PR |
| Peptide | Synthetic Peptide | lanreotide SR |
| Peptide | Synthetic Peptide | larazotide acetate |
| Peptide | Synthetic Peptide | LAT-8881 |
| Peptide | Synthetic Peptide | LBT-1000 |
| Peptide | Synthetic Peptide | LBT-3627 |
| Peptide | Synthetic Peptide | LBT-5001 |
| Peptide | Synthetic Peptide | LBT-6030 |
| Peptide | Synthetic Peptide | LC-002 |
| Peptide | Synthetic Peptide | leconotide |
| Peptide | Synthetic Peptide | leuprolide |
| Peptide | Synthetic Peptide | leuprolide acetate |
| Peptide | Small Molecule; Synthetic | leuprolide acetate + |
| | Peptide | norethindrone |
| Peptide | Synthetic Peptide | leuprolide acetate ER |
| Peptide | Synthetic Peptide | leuprolide acetate PR |
| Peptide | Synthetic Peptide | leuprolide acetate SR |
| Peptide | Synthetic Peptide | leuprorelin acetate PR |
| Peptide | Synthetic Peptide | leuprorelin ER |
| Peptide | Synthetic Peptide | LH-021 |
| Peptide | Synthetic Peptide | LH-024 |
| Peptide | Synthetic Peptide | linaclotide |
| Peptide | Synthetic Peptide | linaclotide DR2 |
| Peptide | Recombinant Peptide | Linjeta |
| Peptide | Recombinant Peptide | liraglutide |
| Peptide | Synthetic Peptide | liraglutide biobetter |
| Peptide | Recombinant Peptide | liraglutide biosimilar |
| Peptide | Synthetic Peptide | livoletide |
| Peptide | Synthetic Peptide | lixisenatide |
| Peptide | Synthetic Peptide | lobradimil |
| Peptide | Synthetic Peptide | LP-003 |
| Peptide | Synthetic Peptide | LTX-315 |
| Peptide | Synthetic Peptide; Vaccine | LTX-315 + tertomotide |
| Peptide | Synthetic Peptide | LTX-401 |
| Peptide | Synthetic Peptide | lutetium Lu 177 dotatate |
| Peptide | Synthetic Peptide | LY-2510924 |
| Peptide | Synthetic Peptide | LY-3143753 |
| Peptide | Synthetic Peptide | LY-3185643 |
| Peptide | Recombinant Peptide | LY-3209590 |
| Peptide | Synthetic Peptide | LY-3305677 |
| Peptide | Synthetic Peptide | LY-355703 |
| Peptide | Recombinant Peptide | LY-900027 |
| Peptide | Recombinant Peptide | Lyumjev |
| Peptide | Synthetic Peptide | M-012 |
| Peptide | Recombinant Peptide | Macrulin |
| Peptide | Synthetic Peptide | MALP-2S |
| Peptide | Synthetic Peptide | mannatide |
| Peptide | Synthetic Peptide | metenkefalin |
| Peptide | Synthetic Peptide | mibenratide |
| Peptide | Synthetic Peptide | mifamurtide |
| Peptide | Synthetic Peptide | mitolactol |
| Peptide | Recombinant Peptide | MOD-1001 |
| Peptide | Recombinant Peptide | MOD-1002 |
| Peptide | Recombinant Peptide | MOD-6030 |
| Peptide | Recombinant Peptide | MOD-6031 |
| Peptide | Synthetic Peptide | motixafortide |
| Peptide | Synthetic Peptide | Motrem |
| Peptide | Synthetic Peptide | MP-3167 |
| Peptide | Synthetic Peptide | MPE-002 |
| Peptide | Recombinant Peptide | MSTMB-103 |
| Peptide | Synthetic Peptide | MT-1002 |
| Peptide | Synthetic Peptide | MTX-1604 |
| Peptide | Synthetic Peptide | MVT-602 |
| Peptide | Synthetic Peptide | NAX-8102 |
| Peptide | Synthetic Peptide | NBI-6024 |
| Peptide | Synthetic Peptide | NBI-69734 |
| Peptide | Synthetic Peptide | NBP-14 |
| Peptide | Synthetic Peptide | nemifitide ditriflutate |
| Peptide | Synthetic Peptide | nepadutant |
| Peptide | Synthetic Peptide | Nephrilin |
| Peptide | Recombinant Peptide | nerinetide |
| Peptide | Synthetic Peptide | Nerofe |
| Peptide | Recombinant Peptide | nesiritide |
| Peptide | Recombinant Peptide | Neucardin |
| Peptide | Recombinant Peptide | NL-005 |
| Peptide | Synthetic Peptide | NLY-001 |
| Peptide | Recombinant Peptide | NN-1952 |
| Peptide | Recombinant Peptide | NN-1954 |
| Peptide | Recombinant Peptide | NN-1955 |
| Peptide | Recombinant Peptide | NN-1956 |
| Peptide | Recombinant Peptide | NN-1965 |
| Peptide | Synthetic Peptide | NN-9277 |
| Peptide | Synthetic Peptide | NN-9423 |
| Peptide | Recombinant Peptide | NN-9513 |
| Peptide | Synthetic Peptide | NN-9536 |
| Peptide | Synthetic Peptide | NN-9747 |
| Peptide | Synthetic Peptide | NN-9775 |
| Peptide | Synthetic Peptide | NN-9838 |
| Peptide | Synthetic Peptide | NN-9931 |
| Peptide | Synthetic Peptide | NNZ-2591 |
| Peptide | Synthetic Peptide | NOV-004 |
| Peptide | Synthetic Peptide | NRP-2945 |
| Peptide | Synthetic Peptide | NRX-1051 |
| Peptide | Recombinant Peptide | NsG-0501 |
| Peptide | Recombinant Peptide | NTRA-2112 |
| Peptide | Recombinant Peptide | NTRA-9620 |
| Peptide | Synthetic Peptide | NX-210 |
| Peptide | Recombinant Peptide | OA-150 |
| Peptide | Synthetic Peptide | OB-3 |
| Peptide | Synthetic Peptide | obinepitide |
| Peptide | Synthetic Peptide | octreotide |
| Peptide | Synthetic Peptide | octreotide acetate |
| Peptide | Synthetic Peptide | octreotide acetate CR |
| Peptide | Synthetic Peptide | octreotide acetate LA |
| Peptide | Synthetic Peptide | octreotide acetate LAR |
| Peptide | Synthetic Peptide | octreotide acetate MAR |
| Peptide | Synthetic Peptide | octreotide acetate microspheres |
| Peptide | Synthetic Peptide | octreotide acetate PR |
| Peptide | Synthetic Peptide | octreotide acetate SR |
| Peptide | Synthetic Peptide | octreotide LA |
| Peptide | Synthetic Peptide | OHR/AVR-118 |
| Peptide | Recombinant Peptide | OI-320GT |
| Peptide | Recombinant Peptide | OI-338GT |
| Peptide | Synthetic Peptide | OK-201 |
| Peptide | Synthetic Peptide | OKI-179 |
| Peptide | Synthetic Peptide | OKI-422 |
| Peptide | Recombinant Peptide | OMO-103 |
| Peptide | Recombinant Peptide | ONCase-PEG |
| Peptide | Synthetic Peptide | ONK-102 |
| Peptide | Synthetic Peptide | ONL-1204 |
| Peptide | Synthetic Peptide | Oratonin |
| Peptide | Synthetic Peptide | orilotimod potassium |
| Peptide | Synthetic Peptide | ornipressin |
| Peptide | Synthetic Peptide | ORTD-1 |
| Peptide | Synthetic Peptide | OXE-103 |
| Peptide | Recombinant Peptide | Oxymera |
| Peptide | Synthetic Peptide | oxyntomodulin |
| Peptide | Synthetic Peptide | oxytocin |
| Peptide | Synthetic Peptide | ozarelix |
| Peptide | Recombinant Peptide | Ozempic |
| Peptide | Synthetic Peptide | P-17 |
| Peptide | Synthetic Peptide | P-28 |
| Peptide | Synthetic Peptide | P-28R |
| Peptide | Synthetic Peptide | P-8 |
| Peptide | Recombinant Peptide | parathyroid hormone |
| Peptide | Synthetic Peptide | pasireotide |
| Peptide | Synthetic Peptide | pasireotide LAR |
| Peptide | Recombinant Peptide | PB-1023 |
| Peptide | Synthetic Peptide | PB-119 |
| Peptide | Synthetic Peptide | PCO-01 |
| Peptide | Synthetic Peptide | PCO-02 |
| Peptide | Synthetic Peptide | PDC-31 |
| Peptide | Recombinant Peptide | PE-0139 |
| Peptide | Synthetic Peptide | PEG Exenatide |
| Peptide | Synthetic Peptide | pegapamodutide |
| Peptide | Synthetic Peptide | pegcetacoplan |
| Peptide | Synthetic Peptide | peginesatide |
| Peptide | Synthetic Peptide | Pegylated Thymalfasin |
| Peptide | Recombinant Peptide | PEN-221 |
| Peptide | Peptide | |
| Peptide | Synthetic Peptide | Peptide T |
| Peptide | Peptide | Peptide to Inhibit Amyloid Beta |
| | | Peptide for Alzheimer's Disease |
| Peptide | Peptide | Peptide to Inhibit GRP-78 for |
| | | Melanoma |
| Peptide | Synthetic Peptide | PHIN-1138 |
| Peptide | Synthetic Peptide | PHIN-837 |
| Peptide | Synthetic Peptide | PI-0824 |
| Peptide | Recombinant Peptide | PI-406 |
| Peptide | Synthetic Peptide | pidotimod |
| Peptide | Synthetic Peptide | PIN-201104 |
| Peptide | Synthetic Peptide | PL-3994 |
| Peptide | Synthetic Peptide | PL-8177 |
| Peptide | Synthetic Peptide | Plannexin |
| Peptide | Synthetic Peptide | plecanatide |
| Peptide | Synthetic Peptide | PLG-0206 |
| Peptide | Synthetic Peptide | plitidepsin |
| Peptide | Synthetic Peptide | PMZ-2123 |
| Peptide | Synthetic Peptide | PN-943 |
| Peptide | Synthetic Peptide | PNT-2002 |
| Peptide | Synthetic Peptide | polyethylene glycol loxenatide |
| | | LAR |
| Peptide | Synthetic Peptide | PP-1420 |
| Peptide | Synthetic Peptide | pramlintide |
| Peptide | Synthetic Peptide | Preimplantation Factor |
| Peptide | Synthetic Peptide | PRI-002 |
| Peptide | Synthetic Peptide | PRI-003 |
| Peptide | Synthetic Peptide | PRI-004 |
| Peptide | Synthetic Peptide | protamine sulfate |
| Peptide | Recombinant Peptide | protamine zinc insulin |
| Peptide | Recombinant Peptide | Protaphane |
| Peptide | Synthetic Peptide | PT-302 |
| Peptide | Synthetic Peptide | PT-320 |
| Peptide | Synthetic Peptide | PT-330 |
| Peptide | Synthetic Peptide | PTG-200 |
| Peptide | Synthetic Peptide | PZ-128 |
| Peptide | Peptide | QUB-3164 |
| Peptide | Recombinant Peptide | rE-4 |
| Peptide | Synthetic Peptide | REC-0438 |
| Peptide | Recombinant Peptide | Recombinant Human Intestinal |
| | | Trefoil Factor |
| Peptide | Recombinant Peptide | Recombinant Peptide 1 to |
| | | Agonize Insulin Receptor for Type |
| | | 1 and Type 2 Diabetes |
| Peptide | Recombinant Peptide | Recombinant Peptide to Agonize |
| | | Calcitonin Gene Related Peptide |
| | | Receptor for Osteoporosis and |
| | | Hypertension |
| Peptide | Recombinant Peptide | Recombinant Peptide to Agonize |
| | | GHRH for Cardiovascular, Central |
| | | Nervous System, Musculoskeletal |
| | | and Metabolic Disorders |
| Peptide | Recombinant Peptide | Recombinant Peptide to Agonize |
| | | GLP1R for Type 2 Diabetes |
| Peptide | Recombinant Peptide | Recombinant Peptide to Agonize |
| | | Insulin receptor for Diabetes |
| Peptide | Recombinant Peptide | Recombinant Peptide to Agonize |
| | | Insulin Receptor for Type 1 and |
| | | Type 2 Diabetes |
| Peptide | Recombinant Peptide | Recombinant Peptide to Agonize |
| | | Insulin Receptor for Type 1 |
| | | Diabetes |
| Peptide | Recombinant Peptide | Recombinant Peptide to Agonize |
| | | Insulin Receptor for Type 2 |
| | | Diabetes |
| Peptide | Recombinant Peptide | Recombinant Peptide to Agonize |
| | | PTH-R for Post Menopausal |
| | | Osteoporosis |
| Peptide | Recombinant Peptide | Recombinant Peptide to Agonize |
| | | PTH1R for Bone Fracture |
| Peptide | Recombinant Peptide | Recombinant Peptide to Agonize |
| | | PTH1R for Hypoparathyroidism |
| Peptide | Recombinant Peptide | Recombinant Peptide to Inhibit |
| | | TNF Alpha for Crohn's Disease, |
| | | Asthma And Metabolic Syndrome |
| Peptide | Recombinant Peptide | Recombinant Peptide-1 to |
| | | Activate GLP-1 for Type 2 |
| | | Diabetes |
| Peptide | Recombinant Peptide | Recombinant Peptides 6 to |
| | | Agonize Insulin Receptor for Type |
| | | 1 and Type 2 Diabetes |
| Peptide | Recombinant Peptide | Recombinant Peptides to Activate |
| | | GLP-1 for Type-2 Diabetes |
| Peptide | Recombinant Peptide | Recombinant Peptides to Agonize |
| | | Insulin Receptor for Type 1 and |
| | | Type 2 Diabetes |
| Peptide | Recombinant Peptide | Recombinant Peptides to Agonize |
| | | MFN2 for Charcot Marie Tooth |
| | | Disease Type IIA and |
| | | Hypertrophic Cardiomyopathy |
| Peptide | Synthetic Peptide | Reg-O3 |
| Peptide | Synthetic Peptide | relamorelin |
| Peptide | Synthetic Peptide | reltecimod sodium |
| Peptide | Recombinant Peptide | Rescue-G |
| Peptide | Synthetic Peptide | RGN-352 |
| Peptide | Recombinant Peptide | Rh-RGD-Hirudin |
| Peptide | Synthetic Peptide | risuteganib |
| Peptide | Synthetic Peptide | romidepsin |
| Peptide | Synthetic Peptide | RPI-78M |
| Peptide | Synthetic Peptide | RPI-MN |
| Peptide | Recombinant Peptide | RTP-025 |
| Peptide | Synthetic Peptide | rusalatide acetate |
| Peptide | Synthetic Peptide | Rybelsus |
| Peptide | Recombinant Peptide | SAR-161271 |
| Peptide | Synthetic Peptide | SAR-425899 |
| Peptide | Recombinant Peptide | Saxenda |
| Peptide | Synthetic Peptide | SBI-1301 |
| Peptide | Synthetic Peptide | SBT-20 |
| Peptide | Synthetic Peptide | SBT-272 |
| Peptide | Synthetic Peptide | SCO-094 |
| Peptide | Synthetic Peptide | SER-130 |
| Peptide | Synthetic Peptide | setmelanotide |
| Peptide | Synthetic Peptide | setmelanotide ER |
| Peptide | Synthetic Peptide | SGX-943 |
| Peptide | Recombinant Peptide | somatostatin |
| Peptide | Recombinant Peptide | somatrem |
| Peptide | Recombinant Peptide | somatrogon |
| Peptide | Synthetic Peptide | SORC-13 |
| Peptide | Synthetic Peptide | sovateltide |
| Peptide | Synthetic Peptide | SRI-31277 |
| Peptide | Synthetic Peptide | STR-324 |
| Peptide | Synthetic Peptide | Synthetic Peptide 1 to Inhibit |
| | | PD-L1 for Oncology |
| Peptide | Synthetic Peptide | Synthetic Peptide for Dengue |
| Peptide | Synthetic Peptide | Synthetic Peptide for Huntington |
| | | Disease |
| Peptide | Synthetic Peptide | Synthetic Peptide for Oncology |
| Peptide | Synthetic Peptide | Synthetic Peptide for Zika Virus |
| | | Infection |
| Peptide | Synthetic Peptide | Synthetic Peptide to Agonize |
| | | GLP1R for Type 2 Diabetes |
| Peptide | Synthetic Peptide | Synthetic Peptide to Agonize |
| | | Insulin Receptor for Type 2 |
| | | Diabetes |
| Peptide | Synthetic Peptide | Synthetic Peptide to Inhibit Alpha |
| | | Synuclein for Parkinson's Disease |
| Peptide | Synthetic Peptide | Synthetic Peptide to Inhibit |
| | | Connexin 43 for Optic Neuropathy |
| Peptide | Synthetic Peptide | Synthetic Peptide to Inhibit ELK1 |
| | | for Central Nervous System |
| | | Disorders |
| Peptide | Synthetic Peptide | Synthetic Peptide to Inhibit |
| | | PCSK9 for Hypercholesterolemia |
| Peptide | Synthetic Peptide | Synthetic Peptide to Inhibit SOD1 |
| | | for Amyotrophic Lateral Sclerosis |
| Peptide | Synthetic Peptide | Synthetic Peptide to Inhibit Tau |
| | | for Tauopathies |
| Peptide | Synthetic Peptide | Synthetic Peptide to Inhibit TNF- |
| | | Alpha for Rheumatoid Arthritis |
| Peptide | Synthetic Peptide | Synthetic Peptide to Inhibit |
| | | VEGFD for Oncology |
| Peptide | Synthetic Peptide | Synthetic Peptide to Modulate |
| | | GHSR for Chronic Kidney |
| | | Disease |
| Peptide | Synthetic Peptide | Synthetic Peptide to Target |
| | | CCKBR for Medullary Thyroid |
| | | Cancer |
| | | Synthetic Peptide to Target |
| | | Somatostatin Receptor for |
| | | Neuroendocrine |
| Peptide | Synthetic Peptide | Gastroenteropancreatic Tumors |
| Peptide | Synthetic Peptide | Synthetic Peptide to Target |
| | | Somatostatin Receptor for |
| | | Neuroendocrine Tumors |
| Peptide | Synthetic Peptide | Synthetic Peptides to Activate |
| | | TMEM173 for Oncology |
| Peptide | Synthetic Peptide | Synthetic Peptides to Agonize |
| | | DOR1 and MOR1 for Irritable |
| | | Bowel Syndome with Diarrhea |
| Peptide | Synthetic Peptide | Synthetic Peptides to Agonize |
| | | GLP1R for Type 2 Diabetes |
| Peptide | Synthetic Peptide | Synthetic Peptides to Agonize |
| | | TLR for Oncology |
| Peptide | Synthetic Peptide | Synthetic Peptides to Antagonize |
| | | CXCR7 for Oncology |
| Peptide | Synthetic Peptide | Synthetic Peptides to Inhibit Beta |
| | | Catenin for Oncology |
| Peptide | Synthetic Peptide | Synthetic Peptides to Inhibit |
| | | Complement C3 for Unspecified |
| | | Indication |
| Peptide | Synthetic Peptide | Synthetic Peptides to Inhibit |
| | | Cyclin E for Oncology |
| Peptide | Synthetic Peptide | Synthetic Peptides to Inhibit |
| | | CyclinA/CDK2 for Oncology |
| Peptide | Synthetic Peptide | Synthetic Peptides to Inhibit |
| | | DRB1 for Multiple Sclerosis |
| Peptide | Synthetic Peptide | Synthetic Peptides to Inhibit E1 |
| | | and E2 Glycoprotein for HCV |
| Peptide | Synthetic Peptide | Synthetic Peptides to Inhibit |
| | | Factor D for Geographic Atrophy, |
| | | Paroxysmal Nocturnal |
| | | Hemoglobinuria and Renal |
| | | Disease |
| Peptide | Synthetic Peptide | Synthetic Peptides to Inhibit |
| | | Glycoprotein VI for Thrombosis |
| Peptide | Synthetic Peptide | Synthetic Peptides to Inhibit |
| | | MCL1 for Oncology |
| Peptide | Synthetic Peptide | Synthetic Peptides to Inhibit |
| | | SMURF2 for Fibrosis and |
| | | Oncology |
| Peptide | Synthetic Peptide | Synthetic Peptides to Inhibit |
| | | TREM-1 for Oncology, Sepsis, |
| | | Rheumatoid Arthritis, Retinopathy |
| | | Of Prematurity and Hemorrhagic |
| | | Shock |
| Peptide | Recombinant Peptide | T-0005 |
| Peptide | Synthetic Peptide | T-20K |
| Peptide | Recombinant Peptide | TAC-201 |
| Peptide | Synthetic Peptide | Tatbeclin-1 |
| Peptide | Recombinant Peptide | TBR-760 |
| Peptide | Synthetic Peptide | TCANG-05 |
| Peptide | Synthetic Peptide | TCMCB-07 |
| Peptide | Recombinant Peptide | teduglutide |
| Peptide | Synthetic Peptide | teicoplanin |
| Peptide | Recombinant Peptide | teriparatide |
| Peptide | Recombinant Peptide | teriparatide acetate |
| Peptide | Recombinant Peptide | teriparatide biosimilar |
| Peptide | Synthetic Peptide | terlipressin |
| Peptide | Synthetic Peptide | tesamorelin acetate |
| Peptide | Synthetic Peptide | THR-149 |
| Peptide | Synthetic Peptide | thymalfasin |
| Peptide | Recombinant Peptide | tifacogin |
| Peptide | Synthetic Peptide | tirzepatide |
| Peptide | Synthetic Peptide | TPX-100 |
| Peptide | Synthetic Peptide | triptorelin |
| Peptide | Synthetic Peptide | triptorelin acetate |
| Peptide | Synthetic Peptide | triptorelin acetate ER |
| Peptide | Synthetic Peptide | triptorelin acetate SR |
| Peptide | Synthetic Peptide | triptorelin pamoate |
| Peptide | Synthetic Peptide | triptorelin pamoate ER |
| Peptide | Synthetic Peptide | triptorelin SR |
| Peptide | Synthetic Peptide | TXA-127 |
| Peptide | Synthetic Peptide | TXA-302 |
| Peptide | Recombinant Peptide | UGP-281 |
| Peptide | Recombinant Peptide | UGP-302 |
| Peptide | Recombinant Peptide | Ultratard |
| Peptide | Recombinant Peptide | Uni-E4 |
| Peptide | Synthetic Peptide | Upelior |
| Peptide | Synthetic Peptide | V-10 |
| Peptide | Synthetic Peptide | VAL-201 |
| Peptide | Synthetic Peptide | vapreotide acetate |
| Peptide | Synthetic Peptide | vasopressin |
| Peptide | Synthetic Peptide | veldoreotide ER |
| Peptide | Synthetic Peptide | veldoreotide IR |
| Peptide | Synthetic Peptide | VG-1177 |
| Peptide | Recombinant Peptide | VIAcal |
| Peptide | Recombinant Peptide | vosoritide |
| Peptide | Recombinant Peptide | VTCG-15 |
| Peptide | Peptide | XG-402 |
| Peptide | Peptide | XG-404 |
| Peptide | Synthetic Peptide | Y-14 |
| Peptide | Synthetic Peptide | YH-14618 |
| Peptide | Synthetic Peptide | ziconotide |
| Peptide | Synthetic Peptide | zilucoplan |
| Peptide | Recombinant Peptide | Znsulin |
| Peptide | Synthetic Peptide | ZP-10000 |
| Peptide | Synthetic Peptide | ZP-7570 |
| Peptide | Synthetic Peptide | ZT-01 |
| Peptide | Recombinant Peptide | ZT-031 |
| Peptide | Synthetic Peptide | ZYKR-1 |

TABLE 3
Enzymes

| Broad | | |
|---|---|---|
| class | Molecule Type | Drug Name |
| Enzyme | Recombinant Enzyme | AB-002 |
| Enzyme | Recombinant Enzyme | ACN-00177 |
| Enzyme | Recombinant Enzyme | agalsidase alfa |
| Enzyme | Recombinant Enzyme | agalsidase beta |
| Enzyme | Recombinant Enzyme | albutrepenonacog alfa ER |
| Enzyme | Recombinant Enzyme | alglucerase |
| Enzyme | Recombinant Enzyme | alglucosidase alfa |
| Enzyme | Recombinant Enzyme | alteplase |
| Enzyme | Recombinant Enzyme | alteplase biosimilar |
| Enzyme | Enzyme | ancrod |
| Enzyme | Enzyme | anistreplase |
| Enzyme | Recombinant Enzyme | apadamtase alfa |
| Enzyme | Recombinant Enzyme | APN-01 |
| Enzyme | Recombinant Enzyme | asfotase alfa |
| Enzyme | Enzyme | asparaginase |
| Enzyme | Recombinant Enzyme | avalglucosidase alfa |
| Enzyme | Recombinant Enzyme | BCT-100 |
| Enzyme | Recombinant Enzyme | bRESCAP |
| Enzyme | Enzyme | bromelains |
| Enzyme | Recombinant Enzyme | calaspargase pegol |
| Enzyme | Recombinant Enzyme | cerliponase alfa |
| Enzyme | Enzyme | chymopapain |
| Enzyme | Enzyme | chymotrypsin |
| Enzyme | Recombinant Enzyme | coagulation factor IX (recombinant) |
| Enzyme | Recombinant Enzyme | coagulation factor IX (recombinant) |
| | | biosimilar |
| Enzyme | Recombinant Enzyme | coagulation factor VIIa (recombinant) |
| | | biosimilar |
| Enzyme | Recombinant Enzyme | coagulation factor XIII A-subunit |
| | | (recombinant) |
| Enzyme | Enzyme | collagenase clostridium histolyticum |
| Enzyme | Recombinant Enzyme | condoliase |
| Enzyme | Recombinant Enzyme | CP-205 |
| Enzyme | Recombinant Enzyme | CUSA-081 |
| Enzyme | Recombinant Enzyme | dalcinonacog alfa |
| Enzyme | Recombinant Enzyme | elapegademase |
| Enzyme | Recombinant Enzyme | elosulfase alfa |
| Enzyme | Recombinant Enzyme | ERYGEN |
| Enzyme | Recombinant Enzyme | exebacase |
| Enzyme | Recombinant Enzyme | galsulfase |
| Enzyme | Recombinant Enzyme | glucarpidase |
| Enzyme | Enzyme | hemocoagulase |
| Enzyme | Recombinant Enzyme | HGT-1111 |
| Enzyme | Recombinant Enzyme | hRESCAP |
| Enzyme | Recombinant Enzyme | idursulfase |
| Enzyme | Recombinant Enzyme | idursulfase beta |
| Enzyme | Recombinant Enzyme | imiglucerase |
| Enzyme | Recombinant Enzyme | imiglucerase biosimilar |
| Enzyme | Recombinant Enzyme | imlifidase |
| Enzyme | Recombinant Enzyme | JR-141 |
| Enzyme | Recombinant Enzyme | JZP-458 |
| Enzyme | Recombinant Enzyme | KTP-001 |
| Enzyme | Recombinant Enzyme | laronidase |
| Enzyme | Recombinant Enzyme | lesinidase alfa |
| Enzyme | Recombinant Enzyme | Lumizyme |
| Enzyme | Recombinant Enzyme | marzeptacog alfa (activated) |
| Enzyme | Recombinant Enzyme | MEDI-6012 |
| Enzyme | Recombinant Enzyme | MOSS-AGAL |
| Enzyme | Recombinant Enzyme | ocriplasmin |
| Enzyme | Recombinant Enzyme | olipudase alfa |
| Enzyme | Recombinant Enzyme | OT-58 |
| Enzyme | Enzyme | pegademase bovine |
| Enzyme | Recombinant Enzyme | pegadricase |
| Enzyme | Recombinant Enzyme | pegargiminase |
| Enzyme | Recombinant Enzyme | pegaspargase |
| Enzyme | Recombinant Enzyme | pegaspargase biosimilar |
| Enzyme | Recombinant Enzyme | pegcrisantaspase |
| Enzyme | Recombinant Enzyme | pegloticase |
| Enzyme | Recombinant Enzyme | pegunigalsidase alfa |
| Enzyme | Recombinant Enzyme | pegvaliase |
| Enzyme | Recombinant Enzyme | pegvorhyaluronidase alfa |
| Enzyme | Recombinant Enzyme | pegzilarginase |
| Enzyme | Recombinant Enzyme | PF-05230907 |
| Enzyme | Enzyme | PRP |
| Enzyme | Recombinant Enzyme | PT-01 |
| Enzyme | Recombinant Enzyme | ranpirnase |
| Enzyme | Recombinant Enzyme | rasburicase |
| Enzyme | Recombinant Enzyme | |
| Enzyme | Recombinant Enzyme | Recombinant Glucosylceramidase |
| | | Replacement for Type I and Type III |
| | | Gaucher's Disease |
| Enzyme | Recombinant Enzyme | Recombinant Human Alkaline |
| | | Phosphatase Replacement for Acute |
| | | Renal Failure, Hypophosphatasia, |
| | | Sepsis and Ulcerative Colitis |
| Enzyme | Recombinant Enzyme | Recombinant Urate Oxidase Replace- |
| | | ment for Acute Hyperuricemia |
| Enzyme | Recombinant Enzyme | reteplase |
| Enzyme | Recombinant Enzyme | sebelipase alfa |
| Enzyme | Recombinant Enzyme | SHP-610 |
| Enzyme | Enzyme | SOBI-003 |
| Enzyme | Recombinant Enzyme | Spectrila |
| Enzyme | Recombinant Enzyme | staphylokinase |
| Enzyme | Enzyme | streptokinase |
| Enzyme | Recombinant Enzyme | TAK-611 |
| Enzyme | Recombinant Enzyme | taliglucerase alfa |
| Enzyme | Recombinant Enzyme | tenecteplase |
| Enzyme | Recombinant Enzyme | TNX-1300 |
| Enzyme | Recombinant Enzyme | tonabacase |
| Enzyme | Recombinant Enzyme | tralesinidase alfa |
| Enzyme | Enzyme | urokinase |
| Enzyme | Recombinant Enzyme | velaglucerase alfa |
| Enzyme | Recombinant Enzyme | velmanase alfa |
| Enzyme | Recombinant Enzyme | vestronidase alfa |
| Enzyme | Recombinant Enzyme | vonapanitase |
| Enzyme | Recombinant Enzyme | VX-210 |

TABLE 4
Proteins

| Broad | | |
|---|---|---|
| Class | Molecule Type | Drug Name |
| Protein | Recombinant Protein | 3K3A-APC |
| Protein | Fusion Protein | abatacept |
| Protein | Recombinant Protein | abicipar pegol |
| Protein | Protein | abobotulinumtoxin A next generation |
| Protein | Protein | abobotulinumtoxinA |
| Protein | Recombinant Protein | ABY-035 |
| Protein | Recombinant Protein | ABY-039 |
| Protein | Protein | ACP-014 |
| Protein | Recombinant Protein | ACT-101 |
| Protein | Fusion Protein | AD-214 |
| Protein | Fusion Protein | aflibercept |
| Protein | Fusion Protein | aflibercept biosimilar |
| Protein | Fusion Protein | AGT-181 |
| Protein | Fusion Protein | AGT-182 |
| Protein | Fusion Protein | AKR-001 |
| Protein | Protein | Albicin |
| Protein | Recombinant Protein | albiglutide |
| Protein | Fusion Protein | albinterferon alfa-2b |
| Protein | Recombinant Protein | aldafermin |
| Protein | Recombinant Protein | aldesleukin |
| Protein | Fusion Protein | alefacept |
| Protein | Fusion Protein | ALKS-4230 |
| Protein | Fusion Protein | ALPN-101 |
| Protein | Fusion Protein | ALT-801 |
| Protein | Fusion Protein | ALTP-1 |
| Protein | Fusion Protein | ALX-148 |
| Protein | Recombinant Protein | AMRS-001 |
| Protein | Recombinant Protein | anakinra |
| Protein | Recombinant Protein | ancestim |
| Protein | Recombinant Protein | andexanet alfa |
| Protein | Recombinant Protein | antihemophilic factor (recombinant) |
| Protein | Recombinant Protein | antihemophilic factor (human) |
| Protein | Recombinant Protein | antihemophilic factor (recombinant) |
| | | biosimilar |
| Protein | Fusion Protein | antihemophilic factor (recombinant), |
| | | FcFusion protein |
| Protein | Recombinant Protein | antihemophilic factor (recombinant), |
| | | PEGylated |
| Protein | Recombinant Protein | antihemophilic factor (recombinant), |
| | | plasma/albumin free |
| Protein | Recombinant Protein | antihemophilic factor (recombinant), |
| | | plasma/albumin free method |
| Protein | Recombinant Protein | antihemophilic factor (recombinant), |
| | | porcine sequence |
| Protein | Recombinant Protein | antihemophilic factor (recombinant), |
| | | single chain |
| Protein | Recombinant Protein | antithrombin (recombinant) |
| Protein | Fusion Protein | APN-301 |
| Protein | Fusion Protein | APO-010 |
| Protein | Fusion Protein | Aravive-S6 |
| Protein | Fusion Protein | asunercept |
| Protein | Fusion Protein | atacicept |
| Protein | Fusion Protein | ATYR-1923 |
| Protein | Recombinant Protein | ATYR-1940 |
| Protein | Recombinant Protein | AU-011 |
| Protein | Recombinant Protein | aviscumine |
| Protein | Recombinant Protein | avotermin |
| Protein | Fusion Protein | balugrastim |
| Protein | Recombinant Protein | batroxobin |
| Protein | Recombinant Protein | BBT-015 |
| Protein | Recombinant Protein | BCD-131 |
| Protein | Protein | bee venom |
| Protein | Fusion Protein | belatacept |
| Protein | Recombinant Protein | bempegaldesleukin |
| Protein | Protein | beractant |
| Protein | Recombinant Protein | BG-8962 |
| Protein | Fusion Protein | bintrafusp alfa |
| Protein | Recombinant Protein | BIO89-100 |
| Protein | Fusion Protein | BIVV-001 |
| Protein | Fusion Protein | blisibimod |
| Protein | Recombinant Protein; | boceprevir + peginterferon alfa-2b + |
| | Small Molecule | ribavirin |
| Protein | Protein | botulinum toxin type A |
| Protein | Protein | BXQ-350 |
| Protein | Protein | C1 esterase inhibitor (human) |
| Protein | Recombinant Protein | C1-esterase inhibitor |
| Protein | Protein | Cadisurf |
| Protein | Recombinant Protein | Cardiotrophin-1 |
| Protein | Protein | CB-24 |
| Protein | Fusion Protein | CD-24Fc |
| Protein | Recombinant Protein | CDX-301 |
| Protein | Recombinant Protein | cepeginterferon alfa-2b |
| Protein | Recombinant Protein | CER-001 |
| Protein | Recombinant Protein | CG-100 |
| Protein | Recombinant Protein | CG-367 |
| Protein | Recombinant Protein | choriogonadotropin alfa |
| Protein | Recombinant Protein | chorionic gonadotropin |
| Protein | Recombinant Protein | CIGB-128 |
| Protein | Protein | CIGB-845 |
| Protein | Recombinant Protein | cimaglermin alfa |
| Protein | Recombinant Protein | cintredekin besudotox |
| Protein | Fusion Protein | coagulation factor IX (recombinant), Fc |
| | | fusion protein |
| Protein | Recombinant Protein | coagulation factor IX (recombinant), |
| | | glycopegylated |
| Protein | Recombinant Protein | coagulation Factor VIIa (Recombinant) |
| Protein | Recombinant Protein | coagulation factor VIII (recombinant) |
| | | biosimilar |
| Protein | Fusion Protein | conbercept |
| Protein | Recombinant Protein | conestat alfa |
| Protein | Recombinant Protein | corifollitropin alfa |
| Protein | Fusion Protein | CSL-689 |
| Protein | Recombinant Protein | CSL-730 |
| Protein | Fusion Protein | CTI-1601 |
| Protein | Fusion Protein | CUE-101 |
| Protein | Recombinant Protein | CVBT-141A |
| Protein | Recombinant Protein | CVBT-141C |
| Protein | Recombinant Protein | CYT-6091 |
| Protein | Recombinant Protein | CYT-99007 |
| Protein | Recombinant Protein | Cyto-012 |
| Protein | Recombinant Protein | dapiclermin |
| Protein | Recombinant Protein | darbepoetin alfa |
| Protein | Recombinant Protein | darbepoetin alfa biosimilar LA |
| Protein | Recombinant Protein | darbepoetin alfa LA |
| Protein | Fusion Protein | darleukin |
| Protein | Fusion Protein | daromun |
| Protein | Fusion Protein | dazodalibep |
| Protein | Fusion Protein | Dekavil |
| Protein | Recombinant Protein | denenicokin |
| Protein | Fusion Protein | denileukin diftitox |
| Protein | Protein | Dextran-Hemoglobin |
| Protein | Fusion Protein | DI-Leu16-IL2 |
| Protein | Recombinant Protein | dianexin |
| Protein | Recombinant Protein | dibotermin alfa |
| Protein | Recombinant Protein | DM-199 |
| Protein | Fusion Protein | DMX-101 |
| Protein | Fusion Protein | DNL-310 |
| Protein | Recombinant Protein | drotrecogin alfa (activated) |
| Protein | Fusion Protein | DSP-107 |
| Protein | Fusion Protein | dulaglutide |
| Protein | Recombinant Protein | ecallantide |
| Protein | Recombinant Protein | ECI-301 |
| Protein | Recombinant Protein | edodekin alfa |
| Protein | Fusion Protein | efavaleukin alfa |
| Protein | Fusion Protein | efineptakin alfa |
| Protein | Recombinant Protein | efinopegdutide |
| Protein | Recombinant Protein | eflapegrastim |
| Protein | Recombinant Protein | efpegsomatropin |
| Protein | Fusion Protein | eftansomatropin alfa |
| Protein | Fusion Protein | eftilagimod alfa |
| Protein | Fusion Protein | eftozanermin alfa |
| Protein | Recombinant Protein | empegfilgrastim |
| Protein | Recombinant Protein | entolimod |
| Protein | Fusion Protein | envafolimab |
| Protein | Recombinant Protein | epidermal growth factor |
| Protein | Recombinant Protein | epoetin alfa |
| Protein | Recombinant Protein | epoetin alfa Long Acting |
| Protein | Recombinant Protein | epoetin beta |
| Protein | Recombinant Protein | epoetin delta |
| Protein | Recombinant Protein | epoetin theta |
| Protein | Recombinant Protein | epoetin zeta |
| Protein | Recombinant Protein | ErepoXen |
| Protein | Fusion Protein | etanercept |
| Protein | Fusion Protein | etanercept biosimilar |
| Protein | Protein | EYS-611 |
| Protein | Fusion Protein | F-627 |
| Protein | Fusion Protein | F-652 |
| Protein | Fusion Protein | F-899 |
| Protein | Recombinant Protein | Fertavid |
| Protein | Fusion Protein | fexapotide triflutate |
| Protein | Fusion Protein | fibromun |
| Protein | Recombinant Protein | filgrastim |
| Protein | Recombinant Protein | follicle stimulating hormone |
| Protein | Recombinant Protein | follitropin alfa |
| Protein | Recombinant Protein | follitropin beta |
| Protein | Recombinant Protein | follitropin delta |
| Protein | Recombinant Protein | FOV-2501 |
| Protein | Recombinant Protein | FSH-GEX |
| Protein | Fusion Protein | Fusion Protein to Antagonize EGFR for |
| | | Glioblastoma Multiforme and Malignant |
| | | Glioma |
| Protein | Fusion Protein | Fusion Protein to Inhibit CD25 for |
| | | Oncology |
| Protein | Fusion Protein | Fusion Protein to Target Mesothelin for |
| | | Oncology |
| Protein | Recombinant Protein | GEM-ONJ |
| Protein | Protein | gemibotulinumtoxin A |
| Protein | Recombinant Protein | GR-007 |
| Protein | | GT-0486 |
| Protein | Fusion Protein | GXG-3 |
| Protein | Fusion Protein | GXG-6 |
| Protein | Protein | Haegarda |
| Protein | Protein | haptoglobin (human) |
| Protein | Fusion Protein | HB-0021 |
| Protein | Protein | hemoglobin glutamer-250 (bovine) |
| Protein | Protein | hemoglobin raffimer |
| Protein | Recombinant Protein | HER-902 |
| Protein | Recombinant Protein | HM-15912 |
| Protein | Fusion Protein | HX-009 |
| Protein | Fusion Protein | IBI-302 |
| Protein | Fusion Protein | ICON-1 |
| Protein | Fusion Protein | IGN-002 |
| Protein | Fusion Protein | IMCF-106C |
| Protein | Fusion Protein | IMM-01 |
| Protein | Protein | INB-03 |
| Protein | Fusion Protein | inbakicept |
| Protein | Fusion Protein | INBRX-101 |
| Protein | Protein | incobotulinumtoxin A |
| Protein | Protein | INS-068 |
| Protein | Protein | interferon alfa |
| Protein | Recombinant Protein | interferon alfa-2a |
| Protein | Recombinant Protein | interferon alfa-2b |
| Protein | Recombinant Protein; | interferon alfa-2b + ribavirin |
| | Small Molecule | |
| Protein | Recombinant Protein | interferon alfa-n3 |
| Protein | Recombinant Protein | interferon alfacon-1 |
| Protein | Recombinant Protein | interferon alpha-n1 |
| Protein | Recombinant Protein | interferon beta-1a |
| Protein | Recombinant Protein | interferon beta-1b |
| Protein | Recombinant Protein | interferon gamma-1b |
| Protein | Recombinant Protein | IRL-201805 |
| Protein | Recombinant Protein | KAN-101 |
| Protein | Fusion Protein | KD-033 |
| Protein | Protein | KER-050 |
| Protein | Fusion Protein | KH-903 |
| Protein | Recombinant Protein | KMRC-011 |
| Protein | Recombinant Protein | Kovaltry |
| Protein | Recombinant Protein | KP-100IT |
| Protein | Recombinant Protein | lenograstim |
| Protein | Recombinant Protein | lepirudin |
| Protein | Fusion Protein | LEVI-04 |
| Protein | Recombinant Protein | liatermin |
| Protein | Fusion Protein | LIB-003 |
| Protein | Recombinant Protein | lipegfilgrastim |
| Protein | Fusion Protein | LMB-100 |
| Protein | Recombinant Protein | lonapegsomatropin |
| Protein | Protein | LTI-01 |
| Protein | Fusion Protein | luspatercept |
| Protein | Recombinant Protein | lusupultide |
| Protein | Recombinant Protein | lutropin alfa |
| Protein | Recombinant Protein | M-9241 |
| Protein | Fusion Protein | MDNA-55 |
| Protein | Recombinant Protein | mecasermin |
| Protein | Recombinant Protein | mecasermin rinfabate |
| Protein | Protein | Menopur |
| Protein | Protein | menotropins |
| Protein | Recombinant Protein | methoxy polyethylene glycol-epoetin |
| | | beta |
| Protein | Recombinant Protein | metreleptin |
| Protein | Recombinant Protein | MG-29 |
| Protein | Recombinant Protein | molgramostim |
| Protein | Recombinant Protein | MP-0250 |
| Protein | Recombinant Protein | MP-0274 |
| Protein | Recombinant Protein | MP-0310 |
| Protein | Fusion Protein | MT-3724 |
| Protein | Recombinant Protein | Multiferon |
| Protein | Recombinant Protein | Multikine |
| Protein | Recombinant Protein | NA-704 |
| Protein | Fusion Protein | naptumomab estafenatox |
| Protein | Recombinant Protein | NE-180 |
| Protein | Recombinant Protein | nepidermina |
| Protein | Recombinant Protein | NGM-386 |
| Protein | Recombinant Protein | NGM-395 |
| Protein | Fusion Protein | NGR-hTNF |
| Protein | Protein | nivobotulinumtoxin A |
| Protein | Fusion Protein | NIZ-985 |
| Protein | Recombinant Protein | NKTR-255 |
| Protein | Recombinant Protein | NKTR-358 |
| Protein | Recombinant Protein | NL-201 |
| Protein | Recombinant Protein | NMIL-121 |
| Protein | Recombinant Protein | NN-7128 |
| Protein | Protein | NN-9215 |
| Protein | Recombinant Protein | NN-9499 |
| Protein | Recombinant Protein | novaferon |
| Protein | Fusion Protein | NPT-088 |
| Protein | Fusion Protein | NPT-189 |
| Protein | Protein | NStride APS |
| Protein | Fusion Protein | olamkicept |
| Protein | Protein | onabotulinumtoxin A |
| Protein | Protein | onabotulinumtoxinA biosimilar |
| Protein | Protein | onabotulinumtoxinA SR |
| Protein | Recombinant Protein | Oncolipin-IT |
| Protein | Recombinant Protein | OPK-88005 |
| Protein | Fusion Protein | oportuzumab monatox |
| Protein | Recombinant Protein | oprelvekin |
| Protein | Recombinant Protein | OPT-302 |
| Protein | Protein | OTO-413 |
| Protein | Fusion Protein | OXS-1550 |
| Protein | Fusion Protein | OXS-3550 |
| Protein | Recombinant Protein | palifermin |
| Protein | Fusion Protein | PB-1046 |
| Protein | Recombinant Protein | PBB-8-IN |
| Protein | Recombinant Protein | PD-1 Antagonist + ropeginterferon |
| | | alfa-2b |
| Protein | Recombinant Protein | PEG-EPO |
| Protein | Recombinant Protein | pegbelfermin |
| Protein | Recombinant Protein | pegfilgrastim |
| Protein | Recombinant Protein | pegilodecakin |
| Protein | Recombinant Protein | peginterferon alfa-2a |
| Protein | Recombinant Protein; | peginterferon alfa-2a + ribavirin |
| | Small Molecule | |
| Protein | Recombinant Protein | peginterferon alfa-2b |
| Protein | Recombinant Protein; | peginterferon alfa-2b + ribavirin |
| | Small Molecule | |
| Protein | Recombinant Protein | peginterferon beta-1a |
| Protein | Recombinant Protein | peginterferon lambda-1a |
| Protein | Recombinant Protein | pegvisomant |
| Protein | Fusion Protein | PF-06755347 |
| Protein | Recombinant Protein | PIN-2 |
| Protein | Protein | plasminogen (human) |
| Protein | Protein | plasminogen (human) 1 |
| Protein | Fusion Protein | PR-15 |
| Protein | Protein | prabotulinumtoxin A biosimilar |
| Protein | Recombinant Protein | Prolanta |
| Protein | Recombinant Protein | PRS-080 |
| Protein | Fusion Protein | PRS-343 |
| Protein | Recombinant Protein | PRT-01 |
| Protein | Protein | PRTX-100 |
| Protein | Fusion Protein | PT-101 |
| Protein | Recombinant Protein | PTR-01 |
| Protein | Recombinant Protein | PTX-9908 |
| Protein | Fusion Protein | QL-1207 |
| Protein | Fusion Protein | RC-28 |
| Protein | Recombinant Protein | RecD-1 |
| Protein | Recombinant Protein | Recombinant Factor VIII Replacement |
| | | for Hemophilia A |
| Protein | Recombinant Protein | Recombinant Plasma Gelsolin |
| | | Replacement for Infectious Disease |
| Protein | Recombinant Protein | Recombinant Protein to Agonize |
| | | BMPR1A, BMPR1B and BMPR2 for |
| | | Colorectal Cancer and Glioblastoma |
| | | Multiforme |
| Protein | Recombinant Protein | Recombinant Protein to Agonize |
| | | IFNAR1 and IFNAR2 for Oncology |
| Protein | Recombinant Protein | Recombinant Protein to Inhibit CD13 |
| | | for Lymphoma and Solid Tumor |
| Protein | Recombinant Protein | Recombinant Protein to Inhibit |
| | | Coagulation Factor XIV for Hemophilia |
| | | A and Hemophilia B |
| Protein | Recombinant Protein | Recombinant Protein to Target FLT1 |
| | | for Pre-Eclampsia |
| Protein | Fusion Protein | reveglucosidase alfa |
| Protein | Fusion Protein | RG-6290 |
| Protein | Fusion Protein | RG-7461 |
| Protein | Fusion Protein | RG-7835 |
| Protein | Recombinant Protein | RG-7880 |
| Protein | Fusion Protein | rilonacept |
| Protein | Protein | rimabotulinumtoxin B |
| Protein | Recombinant Protein | RMC-035 |
| Protein | Fusion Protein | RO-7227166 |
| Protein | Fusion Protein | romiplostim |
| Protein | Fusion Protein | romiplostim biosimilar |
| Protein | Recombinant Protein | ropeginterferon alfa-2b |
| Protein | Recombinant Protein | RP-72 |
| Protein | Fusion Protein | RPH-104 |
| Protein | Fusion Protein | RPH-203 |
| Protein | Fusion Protein | RSLV-132 |
| Protein | Protein | RT-002 |
| Protein | Fusion Protein | SAL-016 |
| Protein | Recombinant Protein | Sanguinate |
| Protein | Fusion Protein | SAR-442085 |
| Protein | Recombinant Protein | sargramostim |
| Protein | Recombinant Protein | SC-0806 |
| Protein | Fusion Protein | SCB-313 |
| Protein | Recombinant Protein | serelaxin |
| Protein | Fusion Protein | SFR-9216 |
| Protein | Recombinant Protein | SHP-608 |
| Protein | Fusion Protein | SHR-1501 |
| Protein | Recombinant Protein | SIM-0710 |
| Protein | Fusion Protein | SL-279252 |
| Protein | Fusion Protein | SOC-101 |
| Protein | Recombinant Protein | somapacitan |
| Protein | Recombinant Protein | somatropin |
| Protein | Recombinant Protein | somatropin pegol |
| Protein | Recombinant Protein | somatropin PR |
| Protein | Recombinant Protein | somatropin SR |
| Protein | Recombinant Protein | somavaratan |
| Protein | Fusion Protein | sotatercept |
| Protein | Recombinant Protein | sprifermin |
| Protein | Recombinant Protein | SubQ-8 |
| Protein | Recombinant Protein | Sylatron |
| Protein | Fusion Protein | T-Guard |
| Protein | Recombinant Protein | TA-46 |
| Protein | Recombinant Protein | tadekinig alfa |
| Protein | Fusion Protein | tagraxofusp |
| Protein | Protein | TAK-101 |
| Protein | Fusion Protein | TAK-169 |
| Protein | Fusion Protein | TAK-573 |
| Protein | Fusion Protein | TAK-671 |
| Protein | Fusion Protein | talditercept alfa |
| Protein | Recombinant Protein | tasonermin |
| Protein | Recombinant Protein | TBI-302 |
| Protein | Recombinant Protein | tbo-filgrastim |
| Protein | Fusion Protein | tebentafusp |
| Protein | Fusion Protein | Teleukin |
| Protein | Fusion Protein | telitacicept |
| Protein | Fusion Protein | TG-103 |
| Protein | Recombinant Protein | THOR-707 |
| Protein | Recombinant Protein | thrombomodulin alfa |
| Protein | Recombinant Protein | thrombopoietin |
| Protein | Recombinant Protein | thyrotropin alfa |
| Protein | Recombinant Protein | tiprelestat |
| Protein | Recombinant Protein | topsalysin |
| Protein | Recombinant Protein | TransMID |
| Protein | Fusion Protein | trebananib |
| Protein | Fusion Protein | TTI-621 |
| Protein | Fusion Protein | TTI-622 |
| Protein | Fusion Protein | tucotuzumab celmoleukin |
| Protein | Recombinant Protein | TVN-102 |
| Protein | Fusion Protein | UCHT-1 |
| Protein | Fusion Protein | VAL-1221 |
| Protein | Fusion Protein | Vas-01 |
| Protein | Recombinant Protein | vatreptacog alfa (activated) |
| Protein | Fusion Protein | VB-4847 |
| Protein | Recombinant Protein | von willebrand factor (recombinant) |
| Protein | Fusion Protein | YSPSL |
| Protein | Fusion Protein | ziv-aflibercept |
| Protein | Protein | ZK-001 |
| Protein | Recombinant Protein | Zorbtive |

### B. Enzymes

The exogenous peptide, polypeptide, or protein may be an enzyme, e.g., an enzyme that catalyzes a biological reaction that is of use in the prevention or treatment of a condition or a disease, the prevention or treatment of a pathogen infection, the diagnosis of a disease, or the diagnosis of a disease or condition.

The enzyme may be a recombination enzyme, e.g., a Cre recombinase enzyme. In some embodiments, the Cre recombinase enzyme is delivered by a complex lipid formulation or a modified PMP to a cell comprising a Cre reporter construct.

The enzyme may be an editing enzyme, e.g., a gene editing enzyme. In some embodiments, the gene editing enzyme is, e.g., a component of a CRISPR-Cas system (e.g., a Cas9 enzyme), a TALEN, or a zinc finger nuclease.

### C. Pathogen Control Agents

The exogenous peptide, polypeptide, or protein may be a pathogen control agent, e.g., a peptide, polypeptide, or protein that is an antibacterial, antifungal, insecticidal, nematicidal, antiparasitic, or virucidal, which is used in human health. In some instances, the complex lipid formulation or the modified PMP formulation described herein includes a peptide, polypeptide, or protein, or functional fragments or derivative thereof, that targets pathways in the pathogen. A complex lipid formulation or a modified PMP formulation including a peptide, polypeptide, or protein as described herein can be administered to a pathogen, a vector thereof, in an amount and for a time sufficient to: (a) reach a target level (e.g., a predetermined or threshold level) of a peptide, polypeptide, or protein concentration; and (b) decrease or eliminate the pathogen. In some instances, a complex lipid formulation or a modified PMP formulation including a peptide, polypeptide, or protein as described herein can be administered to an animal having or at risk of an infection by a pathogen in an amount and for a time sufficient to: (a) reach a target level (e.g., a predetermined or threshold level) of a peptide, polypeptide, or protein concentration in the animal; and (b) decrease or eliminate the pathogen. The peptides, polypeptides, or proteins described herein may be formulated in a complex lipid formulation or a modified PMP formulation for any of the methods described herein, and in certain instances, may be associated with the complex lipid formulation or the modified PMP thereof.

Examples of peptides, polypeptides, or proteins that can be used herein can include an enzyme (e.g., a metabolic recombinase, a helicase, an integrase, a RNAse, a DNAse, or a ubiquitination protein), a pore-forming protein, a signaling ligand, a cell penetrating peptide, a transcription factor, a receptor, an antibody, a nanobody, a gene editing protein (e.g., CRISPR-Cas system, TALEN, or zinc finger), riboprotein, a protein aptamer, or a chaperone.

The complex lipid formulation or the modified PMP formulation described herein may include a bacteriocin. In some instances, the bacteriocin is naturally produced by Gram-positive bacteria, such as Pseudomonas, Streptomyces, Bacillus, Staphylococcus, or lactic acid bacteria (LAB, such as Lactococcus lactis). In some instances, the bacteriocin is naturally produced by Gram-negative bacteria, such as Hafnia alvei, Citrobacter freundii, Klebsiella oxytoca, Klebsiella pneumonia, Enterobacter cloacae, Serratia plymithicum, Xanthomonas campestris, Erwinia carotovora, Ralstonia solanacearum, or Escherichia coli. Exemplary bacteriocins include, but are not limited to, Class I-IV LAB antibiotics (such as lantibiotics), colicins, microcins, and pyocins.

The complex lipid formulation or the modified PMP formulation described herein may include an antimicrobial peptide (AMP). Any AMP suitable for inhibiting a microorganism may be used.

AMPs are a diverse group of molecules, which are divided into subgroups on the basis of their amino acid composition and structure. The AMP may be derived or produced from any organism that naturally produces AMPs, including AMPs derived from plants (e.g., copsin), insects (e.g., mastoparan, poneratoxin, cecropin, moricin, melittin), frogs (e.g., magainin, dermaseptin, aurein), and mammals (e.g., cathelicidins, defensins and protegrins).

### IV. Methods for Producing a Complex Lipid Formulation or a Modified PMP Comprising an Exogenous Polypeptide

Another aspect of the invention relates to a method of producing a complex lipid formulation comprising a plurality of complex lipid particles encapsulating an exogenous peptide, polypeptide, or protein. The method comprises:
extracting at least five lipids from one or more plant sources;
mixing at least two exogenous lipids with the extracted plant lipids to form complex lipid particles; and
loading the complex lipid particles with the exogenous peptide, polypeptide, or protein, wherein the loading causes the exogenous peptide, polypeptide, or protein to be encapsulated by the complex lipid particles, thereby forming the complex lipid formulation.

Additional description on general procedures and exemplary methods to produce complex lipid particles and to encapsulate complex lipid particles with an exogenous peptide, polypeptide, or protein can be found in Examples 1-2.

In another aspect, the disclosure, in general, features a method of producing a modified PMP comprising an exogenous peptide, polypeptide, or protein. The method accordingly comprises (a) providing a solution comprising the exogenous peptide, polypeptide, or protein; and (b) loading the modified PMP with the exogenous peptide, polypeptide, or protein, wherein the loading causes the exogenous peptide, polypeptide, or protein to be encapsulated by the modified PMP.

The exogenous peptide, polypeptide, or protein may be placed in a solution, e.g., a phosphate-buffered saline (PBS) solution. The exogenous peptide, polypeptide, or protein may or may not be soluble in the solution. If the peptide, polypeptide, or protein is not soluble in the solution, the pH of the solution may be adjusted until the polypeptide is soluble in the solution. Insoluble peptides, polypeptides, or proteins are also useful for loading.

Loading of the complex lipid particles or modified PMP with the exogenous peptide, polypeptide, or protein may comprise or consist of sonication of a solution comprising the exogenous peptide, polypeptide, or protein (e.g., a soluble or insoluble exogenous polypeptide) and a plurality of complex lipid particles or modified PMPs to induce poration of the complex lipid particles or modified PMPs and diffusion of the peptide, polypeptide, or protein into the complex lipid particles or modified PMPs, e.g., sonication according to the protocol described in Wang et al., Nature Comm., 4: 1867, 2013.

Alternatively, loading of the complex lipid particles or modified PMP with the exogenous peptide, polypeptide, or protein may comprise or consist of electroporation of a solution comprising the exogenous peptide, polypeptide, or protein (e.g., a soluble or insoluble exogenous polypeptide) and a plurality of complex lipid particles or modified PMPs, e.g., electroporation according to the protocol described in Wahlgren et al., Nucl. Acids. Res., 40(17), e130, 2012.

Alternatively, a small amount of a detergent (e.g., saponin) can be added to increase loading of the exogenous peptide, polypeptide, or protein into complex lipid particles or modified PMPs, e.g., as described in Fuhrmann et al., J Control Release., 205: 35-44, 2015.

Loading of the complex lipid particles or modified PMP with the exogenous peptide, polypeptide, or protein may comprise or consist of lipid extraction and lipid extrusion. Briefly, plant lipids may be isolated by adding MeOH:CHCl₃ (e.g., 3.75 mL 2:1 (v/v) MeOH:CHCl₃) to PMPs in a PBS solution (e.g., 1 mL of PMPs in PBS) and vortexing the mixture. CHCl₃ (e.g., 1.25 mL) and ddH₂O (e.g., 1.25 mL) are then added sequentially and vortexed. The mixture is then centrifuged at 2,000 r.p.m. for 10 min at 22° C. in glass tubes to separate the mixture into two phases (aqueous phase and organic phase). The organic phase sample containing the plant lipids is dried by heating under nitrogen (2 psi). To load peptide, the isolated plant lipids are mixed with the peptide, polypeptide, or protein solution and passed through a lipid extruder, e.g., according to the protocol from Haney et al., J Control Release, 207: 18-30, 2015.

Plant lipids may also be isolated using methods that isolate additional plant lipid classes, e.g., glycosylinositol phosphorylceramides (GIPCs), as described in Casas et al., Plant Physiology, 170: 367-384, 2016. Briefly, to extract plant lipids including GIPCs, chloroform:methanol:HCl (e.g., 3.5 mL of chloroform:methanol:HCl (200:100:1, v/v/v)) plus butylated hydroxytoluene (e.g., 0.01% (w/v) of butylated hydroxytoluene) is added to and incubated with the PMPs. Next, NaCl (e.g., 2 mL of 0.9% (w/v) NaCl) is added and vortexed for 5 minutes. The sample is then centrifuged to induce the organic phase to aggregate at the bottom of the glass tube, and the organic phase is collected. The upper phase may undergo reextraction with chloroform (e.g., 4 mL of pure chloroform) to isolate lipids. The organic phases are combined and dried. After drying, the aqueous phase is resuspended in water (e.g., 1 mL of pure water) and GIPCs are back-extracted using butanol-1 (e.g., 1 mL of butanol-1) twice. To load exogenous peptide, polypeptide, or protein, the isolated plant lipid phases are mixed with the peptide, polypeptide, or protein solution and are passed through a lipid extruder according to the protocol from Haney et al., J Control Release, 207: 18-30, 2015. Alternatively, lipids may be extracted with methyl tertiary-butyl ether (MTBE):methanol:water plus butylated hydroxytoluene (BHT) or with propan-2-ol:hexane:water.

In some embodiments, isolated GIPCs may be added to isolated plant lipids.

In some embodiments, loading of the complex lipid particle or the modified PMP with the exogenous peptide, polypeptide, or protein comprises sonication and lipid extrusion, as described above.

In some embodiments the exogenous peptide, polypeptide, or protein may be pre-complexed (e.g., using protamine sulfate), or a cationic lipid (e.g., DOTAP) may be added to facilitate encapsulation of negatively charged proteins.

Before use, the loaded complex lipid particles or the loaded modified PMPs may be purified, to remove peptides, polypeptides, or proteins that are not bound to or encapsulated by the complex lipid particle or the modified PMP. Loaded complex lipid particles or loaded modified PMPs may be characterized, and their stability may be tested. Loading of the exogenous peptide, polypeptide, or protein may be quantified by methods known in the art for the quantification of proteins. For example, the Pierce Quantitative Colorimetric Peptide Assay may be used on a small sample of the loaded and unloaded complex lipid particles or modified PMPs, or a Western blot using specific antibodies may be used to detect the exogenous peptide, polypeptide, or protein. Alternatively, peptides, polypeptides, or proteins may be fluorescently labeled, and fluorescence may be used to determine the labeled exogenous peptide, polypeptide, or protein concentration in loaded and unloaded complex lipid particles or modified PMPs. Further descriptions regarding purifications, characterizations, stability, and loading of PMPs may be found in WO 2021/041301, which is incorporated by reference in its entirety.

### V. Therapeutic Methods

The complex lipid formulations or the modified PMP formulations described herein are useful in a variety of therapeutic methods, particularly for the prevention or treatment of a condition or disease or for the prevention or treatment of pathogen infections in animals. The present methods involve delivering the complex lipid formulations or the modified PMP formulations described herein to an animal (e.g., a human).

Provided herein are methods of administering to an animal a complex lipid formulation or a modified PMP formulation disclosed herein. The methods can be useful for preventing or treating a condition or disease or for preventing a pathogen infection in an animal (e.g., a human).

For example, provided herein is a method of treating an animal having a fungal infection, wherein the method includes administering to the animal an effective amount of a complex lipid formulation including a plurality of complex lipid particles or a modified PMP formulation including a plurality of modified PMPs, comprising an exogenous peptide, polypeptide, or protein that is a pathogen control agent, e.g., an antifungal agent. In some instances, the fungal infection is caused by Candida albicans. In some instances, the method decreases or substantially eliminates the fungal infection.

In another aspect, provided herein is a method of treating an animal (e.g., a human) having a bacterial infection, wherein the method includes administering to the animal an effective amount of a complex lipid formulation including a plurality of complex lipid particles or a modified PMP formulation including a plurality of modified PMPs. In some instances, the method includes administering to the animal an effective amount of a complex lipid formulation including a plurality of complex lipid particles or a modified PMP formulation including a plurality of modified PMPs, comprising an exogenous peptide, polypeptide, or protein that is a pathogen control agent, e.g., an antibacterial agent. In some instances, the bacterium is a Streptococcus spp., Pneumococcus spp., Pseudomonas spp., Shigella spp, Salmonella spp., Campylobacter spp., or an Escherichia spp. In some instances, the method decreases or substantially eliminates the bacterial infection. In some instances, the animal is a human, a veterinary animal, or a livestock animal.

The present methods are useful to treat an infection (e.g., as caused by an animal, e.g., human, pathogen) in an animal, which refers to administering treatment to an animal already suffering from a disease to improve or stabilize the animal's condition. This may involve reducing colonization of a pathogen in, on, or around an animal by one or more pathogens (e.g., by about 1%, 2%, 5%, 10%, 20%, 30%, 40%, 50%, 60%, 70%, 80%, 90%, or 100%) relative to a starting amount and/or allow benefit to the individual (e.g., reducing colonization in an amount sufficient to resolve symptoms). In such instances, a treated infection may manifest as a decrease in symptoms (e.g., by about 1%, 2%, 5%, 10%, 20%, 30%, 40%, 50%, 60%, 70%, 80%, 90%, or 100%). In some instances, a treated infection is effective to increase the likelihood of survival of an individual (e.g., an increase in likelihood of survival by about 1%, 2%, 5%, 10%, 20%, 30%, 40%, 50%, 60%, 70%, 80%, 90%, or 100%) or increase the overall survival of a population (e.g., an increase in likelihood of survival by about 1%, 2%, 5%, 10%, 20%, 30%, 40%, 50%, 60%, 70%, 80%, 90%, or 100%). For example, the compositions and methods may be effective to “substantially eliminate” an infection, which refers to a decrease in the infection in an amount sufficient to sustainably resolve symptoms (e.g., for at least 1, 2, 3, 4, 5, 6, 7, 8, 9, 10, 11, or 12 months) in the animal.

The present methods are useful to prevent an infection (e.g., as caused by an animal, e.g., human, pathogen), which refers to preventing an increase in colonization in, on, or around an animal by one or more pathogens (e.g., by about 1%, 2%, 5%, 10%, 20%, 30%, 40%, 50%, 60%, 70%, 80%, 90%, 100%, or more than 100% relative to an untreated animal) in an amount sufficient to maintain an initial pathogen population (e.g., approximately the amount found in a healthy individual), prevent the onset of an infection, and/or prevent symptoms or conditions associated with infection. For example, individuals may receive prophylaxis treatment to prevent a fungal infection while being prepared for an invasive medical procedure (e.g., preparing for surgery, such as receiving a transplant, stem cell therapy, a graft, a prosthesis, receiving long-term or frequent intravenous catheterization, or receiving treatment in an intensive care unit), in immunocompromised individuals (e.g., individuals with cancer, with HIV/AIDS, or taking immunosuppressive agents), or in individuals undergoing long-term antibiotic therapy.

The complex lipid formulation or the modified PMP formulation can be formulated for administration or administered by any suitable method, including, for example, orally, enterally, intravenously, intramuscularly, subcutaneously, intradermally, percutaneously, intraarterially, intraperitoneally, intralesionally, intracranially, intraarticularly, intraprostatically, intrapleurally, intratracheally, intrathecally, intranasally, intravaginally, intrarectally (including intracolonically), topically, intratumorally, peritoneally, subconjunctivally, intravesicularly, mucosally, intrapericardially, intraumbilically, intraocularly, intraorbitally, topically, transdermally, intravitreally (e.g., by intravitreal injection), by eye drop, by inhalation (e.g., by a nebulizer), by injection, by implantation, by infusion, by continuous infusion, by localized perfusion bathing target cells directly, by catheter, by lavage, in cremes, or in lipid compositions. The compositions utilized in the methods described herein can also be administered systemically or locally. The method of administration can vary depending on various factors (e.g., the compound or composition being administered and the severity of the condition, disease, or disorder being treated). In some instances, the complex lipid formulation or the modified PMP formulation is administered intravenously, intramuscularly, subcutaneously, topically, orally, transdermally, intraperitoneally, intraorbitally, by implantation, by inhalation, intrathecally, intraventricularly, or intranasally. Dosing can be by any suitable route, e.g., orally or by injections, such as intravenous or subcutaneous injections, depending in part on whether the administration is brief or chronic. Various dosing schedules including but not limited to single or multiple administrations over various time-points, bolus administration, and pulse infusion are contemplated herein.

The prevention or treatment of an infection described herein (when used alone or in combination with one or more other additional therapeutic agents) will depend on the type of disease to be treated, the severity and course of the disease. Whether the therapeutic agent is administered for preventive or therapeutic purposes will depend on previous therapy, the patient's clinical history, and response to the complex lipid formulation or the modified PMP formulation. The complex lipid formulation or the modified PMP formulation can be, e.g., administered to the patient at one time or over a series of treatments. For repeated administrations over several days or longer, depending on the condition, the treatment would generally be sustained until a desired suppression of disease symptoms occurs or the infection is no longer detectable. Such doses may be administered intermittently, e.g., every week or every two weeks (e.g., such that the patient receives, for example, from about two to about twenty, doses of the complex lipid formulation or the modified PMP formulation. An initial higher loading dose, followed by one or more lower doses may be administered. However, other dosage regimens may be useful. The progress of this therapy is easily monitored by conventional techniques and assays.

In some instances, the amount of the complex lipid formulation or the modified PMP formulation administered to individual (e.g., human) may be in the range of about 0.01 mg/kg to about 5 g/kg (e.g., about 0.01 mg/kg-0.1 mg/kg, about 0.1 mg/kg-1 mg/kg, about 1 mg/kg-10 mg/kg, about 10 mg/kg-100 mg/kg, about 100 mg/kg-1 g/kg, or about 1 g/kg-5 g/kg), of the individual's body weight. In some instances, the amount of the complex lipid formulation or the modified PMP formulation administered to individual (e.g., human) is at least 0.01 mg/kg (e.g., at least 0.01 mg/kg, at least 0.1 mg/kg, at least 1 mg/kg, at least 10 mg/kg, at least 100 mg/kg, at least 1 g/kg, or at least 5 g/kg), of the individual's body weight. The dose may be administered as a single dose or as multiple doses (e.g., 2, 3, 4, 5, 6, 7, or more than 7 doses). In some instances, the complex lipid formulation or the modified PMP formulation administered to the animal may be administered alone or in combination with an additional therapeutic agent or pathogen control agent. The dose of an antibody administered in a combination treatment may be reduced as compared to a single treatment. The progress of this therapy is easily monitored by conventional techniques.

In one aspect, the disclosure features a method for treating diabetes, the method comprising administering to a subject in need thereof an effective amount of a composition comprising a plurality of complex lipid particles or a plurality of modified PMPs, encapsulating one or more exogenous peptides, polypeptides, or proteins. The administration of the plurality of complex lipid particles or the plurality of modified PMPs may lower the blood sugar of the subject. In some embodiments, the exogenous peptide, polypeptide, or protein is insulin, exenatide, semaglutide, or tirzepatide.

### VI. Methods for Treatment of Pathogens or Vectors Thereof

The complex lipid formulation or the modified PMP formulations and related methods described herein are useful to decrease the fitness of an animal pathogen and thereby treat or prevent infections in animals, e.g., humans. Examples of animal pathogens, or vectors thereof, that can be treated with the present compositions or related methods are further described herein.

### A. Fungi

The complex lipid formulation or the modified PMP formulations and related methods can be useful for decreasing the fitness of a fungus, e.g., to prevent or treat a fungal infection in an animal, e.g., a human. Included are methods for delivering a complex lipid formulation or a modified PMP formulation to a fungus by contacting the fungus with the complex lipid formulation or the modified PMP formulation. Additionally, or alternatively, the methods include preventing or treating a fungal infection (e.g., caused by a fungus described herein) in an animal at risk of or in need thereof, by administering to the animal a complex lipid formulation or a modified PMP formulation.

The complex lipid formulation or the modified PMP formulations and related methods are suitable for treatment or preventing of fungal infections in animals, including infections caused by fungi belonging to Ascomycota (Fusarium oxysporum, Pneumocystis jirovecii, Aspergillus spp., Coccidioides immitis/posadasii, Candida albicans), Basidiomycota (Filobasidiella neoformans, Trichosporon), Microsporidia (Encephalitozoon cuniculi, Enterocytozoon bieneusi), Mucoromycotina (Mucor circinelloides, Rhizopus oryzae, Lichtheimia corymbifera).

In some instances, the fungal infection is one caused by a fungus belonging to the phylum Ascomycota, Basidomycota, Chytridiomycota, Microsporidia, or Zygomycota. The fungal infection or overgrowth can include one or more fungal species, e.g., Candida albicans, C. tropicalis, C. parapsilosis, C. glabrata, C. auris, C. krusei, Saccharomyces cerevisiae, Malassezia globose, M. restricta, or Debaryomyces hansenii, Gibberella moniliformis, Alternaria brassicicola, Cryptococcus neoformans, Pneumocystis carinii, P. jirovecii, P. murina, P. oryctolagi, P. wakefieldiae, and Aspergillus clavatus. The fungal species may be considered a pathogen or an opportunistic pathogen.

In some instances, the fungal infection is caused by a fungus in the genus Candida (i.e., a Candida infection). For example, a Candida infection can be caused by a fungus in the genus Candida that is selected from the group consisting of C. albicans, C. glabrata, C. dubliniensis, C. krusei, C. auris, C. parapsilosis, C. tropicalis, C. orthopsilosis, C. guilliermondii, C. rugose, and C. lusitaniae. Candida infections that can be treated by the methods disclosed herein include, but are not limited to candidemia, oropharyngeal candidiasis, esophageal candidiasis, mucosal candidiasis, genital candidiasis, vulvovaginal candidiasis, rectal candidiasis, hepatic candidiasis, renal candidiasis, pulmonary candidiasis, splenic candidiasis, otomycosis, osteomyelitis, septic arthritis, cardiovascular candidiasis (e.g., endocarditis), and invasive candidiasis.

### B. Bacteria

The complex lipid formulation or the modified PMP formulations and related methods can be useful for decreasing the fitness of a bacterium, e.g., to prevent or treat a bacterial infection in an animal, e.g., a human. Included are methods for administering a complex lipid formulation or a modified PMP formulation to a bacterium by contacting the bacteria with the complex lipid formulation or the modified PMP composition. Additionally, or alternatively, the methods include preventing or treating a bacterial infection (e.g., caused by a bacteria described herein) in an animal at risk of or in need thereof, by administering to the animal a complex lipid formulation or a modified PMP formulation.

The complex lipid formulation or the modified PMP formulations and related methods are suitable for preventing or treating a bacterial infection in animals caused by any bacteria described further below. For example, the bacteria may be one belonging to Bacillales (B. anthracis, B. cereus, S. aureus, L. monocytogenes), Lactobacillales (S. pneumoniae, S. pyogenes), Clostridiales (C. botulinum, C. difficile, C. perfringens, C. tetani), Spirochaetales (Borrelia burgdorferi, Treponema pallidum), Chlamydiales (Chlamydia trachomatis, Chlamydophila psittaci), Actinomycetales (C. diphtheriae, Mycobacterium tuberculosis, M. avium), Rickettsiales (R. prowazekii, R. rickettsii, R. typhi, A. phagocytophilum, E. chaffeensis), Rhizobiales (Brucella melitensis), Burkholderiales (Bordetella pertussis, Burkholderia mallei, B. pseudomallei), Neisseriales (Neisseria gonorrhoeae, N. meningitidis), Campylobacterales (Campylobacter jejuni, Helicobacter pylori), Legionellales (Legionella pneumophila), Pseudomonadales (A. baumannii, Moraxella catarrhalis, P. aeruginosa), Aeromonadales (Aeromonas sp.), Vibrionales (Vibrio cholerae, V. parahaemolyticus), Thiotrichales, Pasteurellales (Haemophilus influenzae), Enterobacteriales (Klebsiella pneumoniae, Proteus mirabilis, Yersinia pestis, Y. enterocolitica, Shigella flexneri, Salmonella enterica, E. coli).

The invention may be further represented by the following embodiments:
Embodiment 1. A method for delivering a therapeutic peptide or protein to a human subject in need thereof, the method comprising orally or enterally administering to the human subject a pharmaceutical preparation comprising:
(a) a plurality of complex lipid particles characterized by: (i) comprising at least 10 plant lipids extracted from one or more plant sources; (ii) comprising a sterol exogenous to the one or more plant sources, (iii) comprising a polyethylene glycol (PEG)-conjugated lipid; (iii) containing less than 10% w/w of protein matter endogenous to the one or more plant sources; and (iv) containing less than 10 mol % of exogenous ionizable lipids; and
(b) the therapeutic peptide or protein encapsulated in the complex lipid particles.
   Embodiment 2. The method of embodiment 1, wherein the therapeutic peptide or protein is a hormone or glucagon-like peptide 1 (GLP-1) agonist.
   Embodiment 3. The method of embodiment 2, wherein the therapeutic peptide or protein is insulin, exenatide, semaglutide, or tirzepatide.
   Embodiment 4. The method of embodiment 1, wherein the therapeutic peptide or protein is delivered to a brain tissue in the human subject.
   Embodiment 5. The method of embodiment 1, wherein the complex lipid particle contains ten or more lipids belonging to one or more of the sub-classes selected from the group consisting of acylsterylglycosides, ceramides, digalactosyldiacylglycerols, diacylglyceryl glucuronides, hemibismonoacylglycerophosphates, hexosylceramides, lysophosphatidylcholines, lysophosphatidylethanolamines, monogalactosyldiacylglycerols, phosphatidylcholines, phosphatidylethanolamines, phosphatidylethanols, phosphatidylglycerols, phosphatidylinositols, sulfoquinovosyl diacylglycerols, and sterols.
   Embodiment 6. The method of embodiment 5, wherein the complex lipid particle contains lipids from at least five, at least six, at least seven, at least eight, at least nine, or at least ten different sub-classes.
   Embodiment 7. The method of embodiment 1, wherein the complex lipid particle contains less than 5% w/w of protein matter endogenous to the one or more plant sources.
   Embodiment 8. The method of embodiment 1, wherein the complex lipid particle contains less than 5 mol % of exogenous ionizable lipids.
   Embodiment 9. The method of embodiment 1, wherein at least one of the plant sources is a grapefruit, lemon. dragon fruit, spinach, kale, strawberry, broccoli, or soy.
   Embodiment 10. The method of embodiment 1, wherein the complex lipid particle comprises:
about 85-95% w/w of the plant lipids,
about 5 to 8% w/w of the sterol,
about 1-3.5% w/w the polyethylene glycol (PEG)-lipid conjugate, based on the amounts of total lipids in the complex lipid formulation.
   Embodiment 11. A complex lipid formulation, comprising:
a plurality of complex lipid particles, each complex lipid particle of the plurality comprising at least five lipids extracted from one or more plant sources and at least two exogenous lipids; and
one or more exogenous peptides, polypeptides, or proteins, encapsulated in the complex lipid particles, wherein the complex lipid particles are characterized by one or more of the following characteristics:
i) containing less than 50% w/w of protein matter endogenous to the one or more plant sources; and
ii) containing less than 50 mol % of ionizable lipids.
   Embodiment 12. The complex lipid formulation of embodiment 11, wherein the exogenous peptide, polypeptide, or protein is a therapeutic agent.
   Embodiment 13. The complex lipid formulation of embodiment 11, wherein the exogenous peptide, polypeptide, or protein is an antibody or an antibody fragment.
   Embodiment 14. The complex lipid formulation of embodiment 11, wherein the exogenous peptide, polypeptide, or protein is a hormone.
   Embodiment 15. The complex lipid formulation of embodiment 14, wherein the exogenous peptide, polypeptide, or protein is insulin.
   Embodiment 16. The complex lipid formulation of embodiment 11, wherein the exogenous peptide, polypeptide, or protein is a receptor agonist or a receptor antagonist.
   Embodiment 17. The complex lipid formulation of embodiment 16, wherein the exogenous peptide, polypeptide, or protein is a glucagon-like peptide 1 (GLP-1) agonist.
   Embodiment 18. The complex lipid formulation of embodiment 17, wherein the exogenous peptide, polypeptide, or protein is a exenatide, semaglutide, or tirzepatide.
   Embodiment 19. The complex lipid formulation of embodiment 11, wherein the exogenous peptide, polypeptide, or protein has a size of less than 100 kD.
   Embodiment 20. The complex lipid formulation of embodiment 19, wherein the exogenous peptide, polypeptide, or protein has a size of less than 50 kD.
   Embodiment 21. The complex lipid formulation of embodiment 19, wherein the exogenous peptide, polypeptide, or protein has a size of at least 3 kD.
   Embodiment 22. The complex lipid formulation of embodiment 19, wherein the exogenous peptide, polypeptide, or protein comprises at least 30 amino acid residues.
   Embodiment 23. The complex lipid formulation of embodiment 11, wherein the complex lipid particle contains 5-1000 lipids extracted from one or more plant sources.
   Embodiment 24. The complex lipid formulation of embodiment 11, wherein the complex lipid particle contains at least 10 plant lipids belonging to one or more of the classes selected from the group consisting of glycerolipid, sphingolipid, and sterol.
   Embodiment 25. The complex lipid formulation of embodiment 24, wherein the complex lipid particle contains one or more glycerolipids selected from the group consisting of phospholipids (PL), galactolipids (GL), triacylglycerols (TG), and sulfolipids (SL).
   Embodiment 26. The complex lipid formulation of embodiment 24, wherein the complex lipid particle contains one or more sphingolipids selected from the group consisting of glycosyl inositolphosphoceramides (GIPC), glucosylceramides (GCer), ceramides (Cer), and free long-chain bases (LCB).
   Embodiment 27. The complex lipid formulation of embodiment 24, wherein the complex lipid particle contains one or more phytosterols selected from the group consisting of campesterol, stigmasterol, and sitosterol.
   Embodiment 28. The complex lipid formulation of embodiment 24, wherein the complex lipid particle contains one or more lipids belonging to one or more of the sub-classes selected from the group consisting of acyl diacylglyceryl glucuronides, acylhexosylceramides, acylsterylglycosides, bile acids, acyl carnitines, cholesteryl esters, ceramides, cardiolipins, coenzyme Qs, diacylglycerols, digalactosyldiacylglycerols, diacylglyceryl glucuronides, dilysocardiolipins, fatty acids, fatty acid esters of hydroxyl fatty acids, hemibismonoacylglycerophosphates, hexosylceramides, lysophosphatidic acids, lysophosphatidylcholines, lysophosphatidylethanolamines, N-acyl-lysophosphatidylethanolamines, lysophosphatidylglycerols, lysophosphatidylinositols, lysophosphatidylserines, monogalactosyldiacylglycerols, lysocardiolipins, N-acyl ethanolamines, N-acyl glycines, N-acyl glycyl serines, phosphatidic acids, phosphatidylcholines, phosphatidylethanolamines, phosphatidylethanols, phosphatidylglycerols, phosphatidylinositols, ceramide phosphoinositols, phosphatidylmethanols, phosphatidylserines, steryl esters, stigmasterols, sulfatides, sulfonolipids, sphingomyelins, sulfoquinovosyl diacylglycerols, sterols, and triacylglycerols.
   Embodiment 29. The complex lipid formulation of embodiment 28, wherein the complex lipid particle contains ten or more lipids belonging to one or more of the sub-classes selected from the group consisting of acylsterylglycosides, ceramides, digalactosyldiacylglycerols, diacylglyceryl glucuronides, hemibismonoacylglycerophosphates, hexosylceramides, lysophosphatidylcholines, lysophosphatidylethanolamines, monogalactosyldiacylglycerols, phosphatidylcholines, phosphatidylethanolamines, phosphatidylethanols, phosphatidylglycerols, phosphatidylinositols, sulfoquinovosyl diacylglycerols, and sterols.
   Embodiment 30. The complex lipid formulation of embodiment 28 or 29, wherein the complex lipid particle contains lipids from at least five, at least six, at least seven, at least eight, at least nine, or at least ten different sub-classes.
   Embodiment 31. The complex lipid formulation of embodiment 11, wherein the complex lipid particle contains less than 30% w/w of protein matter endogenous to the one or more plant sources.
   Embodiment 32. The complex lipid formulation of embodiment 31, wherein the complex lipid particle contains less than 5% w/w of protein matter endogenous to the one or more plant sources.
   Embodiment 33. The complex lipid formulation of embodiment 11, wherein the complex lipid particle contains less than 20 mol % of exogenous ionizable lipids.
   Embodiment 34. The complex lipid formulation of embodiment 33, wherein the complex lipid particle contains less than 5 mol % of exogenous ionizable lipids.
   Embodiment 35. The complex lipid formulation of embodiment 11, wherein at least one of the plant sources is a citrus fruit.
   Embodiment 36. The complex lipid formulation of embodiment 35, wherein the citrus fruit is a grapefruit or a lemon.
   Embodiment 37. The complex lipid formulation of embodiment 11, wherein at least one of the plant sources is a non-citrus plant.
   Embodiment 38. The complex lipid formulation of embodiment 37, wherein the non-citrus plant is a dragon fruit, spinach, kale, strawberry, broccoli, or soy.
   Embodiment 39. The complex lipid formulation of embodiment 11, wherein the exogenous lipids comprise a sterol and a polyethylene glycol (PEG)-lipid conjugate.
   Embodiment 40. The complex lipid formulation of embodiment 39, wherein the sterol is cholesterol or sitosterol.
   Embodiment 41. The complex lipid formulation of embodiment 39, wherein the PEG-lipid conjugate is a PEG-DMG or PEG-PE.
   Embodiment 42. The complex lipid formulation of embodiment 39, wherein the PEG-lipid conjugate is a PEG2000-PE, PEG2000-DMG, PEG2000-DSPE, or a derivative thereof.
   Embodiment 43. The complex lipid formulation of embodiment 39, wherein the exogenous lipids further comprise a lipid selected from the group consisting of a fatty acid, a glycerolipid, a glycerophospholipid, a sphingolipid, a second sterol, and an additive synthetic lipid.
   Embodiment 44. The complex lipid formulation of embodiment 39, wherein the complex lipid particle comprises:
about 10-95% w/w of the plant lipids,
about 5 to 60% w/w of the sterol,
about 0.5-15% w/w the polyethylene glycol (PEG)-lipid conjugate, based on the amounts of total lipids in the complex lipid formulation.
   Embodiment 45. The complex lipid formulation of embodiment 44, wherein the complex lipid particle comprises:
about 85-95% w/w of the plant lipids,
about 5 to 8% w/w of the sterol,
about 1-3.5% w/w the polyethylene glycol (PEG)-lipid conjugate, based on the amounts of total lipids in the complex lipid formulation.
   Embodiment 46. The complex lipid formulation of embodiment 21, wherein the complex lipid particles have an average size of less than about 250 nm.
   Embodiment 47. The complex lipid formulation of embodiment 46, wherein the complex lipid particles have an average size of about 100 to 180 nm.
   Embodiment 48. The complex lipid formulation of embodiment 11, wherein the complex lipid particles have a PDI of about 0.1 to about 0.5.
   Embodiment 49. The complex lipid formulation of embodiment 48, wherein the complex lipid particles have a PDI of about 0.2 to about 0.4.
   Embodiment 50. The complex lipid formulation of embodiment 11, wherein the complex lipid particle further comprises one or more cryoprotectants or lyoprotectants.
   Embodiment 51. The complex lipid formulation of embodiment 11, wherein the complex lipid formulation is a lyophilized composition.
   Embodiment 52. The complex lipid formulation of embodiment 11, wherein the complex lipid formulation is a liquid composition.
   Embodiment 53. The complex lipid formulation of embodiment 11, wherein the complex lipid formulation is stable at room temperature, and/or at 4° C. for at least two weeks, without lyophilization.
   Embodiment 54. A pharmaceutical composition comprising the complex lipid formulation according to any one of embodiments 1-53, and a pharmaceutically acceptable vehicle, carrier, or excipient.
   Embodiment 55. The pharmaceutical composition of embodiment 54, wherein the pharmaceutical composition is in a capsule dosage form or a tablet dosage form.
   Embodiment 56. A method for delivering a peptide, polypeptide, or protein to a mammalian cell or a mammal, the method comprising:
contacting the mammalian cell with or administering to the mammal a complex lipid formulation, under conditions sufficient to allow uptake of the complex lipid formulation by the mammalian cell or by the mammal,
wherein the complex lipid formulation comprises:
   a plurality of complex lipid particles, each complex lipid particle of the plurality comprising at least five lipids extracted from one or more plant sources and at least two exogenous lipids, and
   one or more exogenous peptides, polypeptides, or proteins, encapsulated in the complex lipid particles, wherein the complex lipid particles are characterized by one or more of the following characteristics:
   i) containing less than 50% w/w of protein matter endogenous to the one or more plant sources; and
   ii) containing less than 50 mol % of ionizable lipids.
      Embodiment 57. The method of embodiment 56, wherein the mammalian cell is a cell in a human, or the mammal is a human.
      Embodiment 58. The method of embodiment 56, wherein the uptake by the mammalian cell or by the mammal of the exogenous peptide, polypeptide, or protein encapsulated by the complex lipid particles is increased relative to the uptake of the exogenous peptide, polypeptide, or protein not encapsulated by a complex lipid particle.
      Embodiment 59. The method of embodiment 56, wherein the method is for delivering a peptide, polypeptide, or protein to a mammal, and the administration is via oral, enteral, intranasal, intracolonic, intrarectal, or intrajejunal route.
      Embodiment 60. The method of embodiment 56, wherein the mammalian cell is brain cell.
      Embodiment 61. A method for treating or preventing a disease or disorder in a subject for which a therapeutic agent is indicated, the method comprising:
administering to the subject in need thereof an effective amount of a complex lipid formulation comprising:
   a plurality of complex lipid particles, each complex lipid particle of the plurality comprising at least five lipids extracted from one or more plant sources and at least two exogenous lipids, and
   one or more exogenous peptides, polypeptides, or proteins, encapsulated in the complex lipid particles, wherein the complex lipid particles are characterized by one or more of the following characteristics:
   i) containing less than 50% w/w of protein matter endogenous to the one or more plant sources; and
   ii) containing less than 50 mol % of ionizable lipids.
      Embodiment 62. The method of embodiment 61, wherein the administration is via oral, enteral, intranasal, intracolonic, intrarectal, or intrajejunal route.
      Embodiment 63. The method of embodiment 61, wherein the disease is diabetes, and the exogenous peptide, polypeptide, or protein is insulin, exenatide, semaglutide, or tirzepatide.
      Embodiment 64. A method of producing a complex lipid formulation comprising a plurality of complex lipid particles encapsulating an exogenous peptide, polypeptide, or protein, the method comprising:
extracting at least five lipids from one or more plant sources;
mixing at least two exogenous lipids with the extracted plant lipids to form complex lipid particles; and
loading the complex lipid particles with the exogenous peptide, polypeptide, or protein, wherein the loading causes the exogenous peptide, polypeptide, or protein to be encapsulated by the complex lipid particles, thereby forming the complex lipid formulation.
   Embodiment 65. The method of embodiment 64, wherein the lipids are extracted from one or more plant sources by adding to the plant sources an extraction solvent comprising methanol, ethanol, propanol, 1-buthanol, acetonitrile, acetone, dimethylformamide, tetrahydrofuran, dimethyl sulfoxide, methyl tert-butyl ether, chloroform, ethyl acetate, or a mixture thereof.
   Embodiment 66. The method of embodiment 65, wherein the extraction solvent is dichloromethane:methanol, chloroform:methanol, methanol:methyl tert-butyl ether (MTBE), dimethylformamide:methanol; acetonitrile:methanol; acetone:methanol; tetrahydrofuran:methanol; dimethyl sulfoxide:methanol; acetonitrile:ethanol; or ethyl acetate:ethanol.
   Embodiment 67. The method of embodiment 64, wherein the extracting step further comprises reducing or eliminating protein matter endogenous to the one or more plant sources to less than 50% w/w.
   Embodiment 68. The method of embodiment 64, wherein the mixing step is carried out by thin film mixing or microfluidics mixing.
   Embodiment 69. The method of embodiment 64, wherein the exogenous lipids comprise a sterol and a polyethylene glycol (PEG)-lipid conjugate.
   Embodiment 70. The method of embodiment 64, wherein the exogenous lipids do not include an ionizable lipid.
   Embodiment 71. A modified plant messenger pack (PMP) formulation comprising:
one or more PMPs modified with one or more sterols and one or more polyethylene glycol (PEG)-lipid conjugates,
wherein the modified PMPs are formulated with one or more exogenous peptides, polypeptides, or proteins, and wherein the one or more exogenous peptides, polypeptides, or proteins are encapsulated by the modified PMP.
   Embodiment 72. The modified PMP formulation of embodiment 71, wherein the exogenous peptide, polypeptide, or protein is a therapeutic agent.
   Embodiment 73. The modified PMP formulation of embodiment 71, wherein the exogenous peptide, polypeptide, or protein is an enzyme.
   Embodiment 74. The modified PMP formulation of embodiment 73, wherein the enzyme is a recombination enzyme or an editing enzyme.
   Embodiment 75. The modified PMP formulation of embodiment 71, wherein the exogenous peptide, polypeptide, or protein is an antibody or an antibody fragment.
   Embodiment 76. The modified PMP formulation of embodiment 71, wherein the exogenous peptide, polypeptide, or protein is an Fc fusion protein.
   Embodiment 77. The modified PMP formulation of embodiment 71, wherein the exogenous peptide, polypeptide, or protein is a hormone.
   Embodiment 78. The modified PMP formulation of embodiment 77, wherein the exogenous peptide, polypeptide, or protein is insulin.
   Embodiment 79. The modified PMP formulation of embodiment 71, wherein the exogenous peptide, polypeptide, or protein is a receptor agonist or a receptor antagonist.
   Embodiment 80. The modified PMP formulation of any one of embodiments 71-79, wherein the exogenous peptide, polypeptide, or protein has a size of less than 100 kD.
   Embodiment 81. The modified PMP formulation of embodiment 80, wherein the exogenous peptide, polypeptide, or protein has a size of less than 50 kD.
   Embodiment 82. The modified PMP formulation of any one of embodiments 71-79, wherein the exogenous peptide, polypeptide, or protein has a size of at least 5 kD.
   Embodiment 83. The modified PMP formulation of any one of embodiments 71-79, wherein the exogenous peptide, polypeptide, or protein comprises at least 50 amino acid residues.
   Embodiment 84. The modified PMP formulation of any one of embodiments 71-83, wherein the exogenous peptide, polypeptide, or protein has an overall charge that is neutral, or has been modified to have a charge that is neutral.
   Embodiment 85. The modified PMP formulation of any one of embodiments 71-83, wherein the exogenous peptide, polypeptide, or protein has an overall charge that is positive or negative.
   Embodiment 86. The modified PMP formulation of any one of embodiments 71-85, wherein the PMP comprises a purified plant extracellular vesicle (EV), or a segment or extract thereof.
   Embodiment 87. The modified PMP formulation of embodiment 86, wherein the PMP is obtained from a citrus fruit.
   Embodiment 88. The modified PMP formulation of embodiment 87, wherein the citrus fruit is a grapefruit or a lemon.
   Embodiment 89. The modified PMP formulation of any one of embodiments 71-88, wherein the sterol is cholesterol or sitosterol.
   Embodiment 90. The modified PMP formulation of any one of embodiments 71-88, wherein the PEG-lipid conjugate is a C14-PEG2k or C18-PEG2k.
   Embodiment 91. The modified PMP formulation of any one of embodiments 71-88, wherein the PEG-lipid conjugate is a PEG-DMG or PEG-PE.
   Embodiment 92. The modified PMP formulation of any one of embodiments 71-88, wherein the PEG-lipid conjugate is a C18-PEG2000 PE or its derivative.
   Embodiment 93. The modified PMP formulation of any one of embodiments 71-88, wherein the sterol is cholesterol, and the PEG-lipid conjugate is a C18-PEG2000 PE or its derivative.
   Embodiment 94. The modified PMP formulation of embodiment 86, wherein the concentration of the sterol ranges from about 0.5 to 15% w/w, based on the amounts of total lipid extracts.
   Embodiment 95. The modified PMP formulation of embodiment 94, wherein the concentration of the sterol ranges from about 5 to 8% w/w, based on the amounts of total lipid extracts.
   Embodiment 96. The modified PMP formulation of embodiment 86, wherein the concentration of the PEG-lipid conjugate ranges from about 0.5 to 5% w/w, based on the amounts of total lipid extracts.
   Embodiment 97. The modified PMP formulation of embodiment 96, wherein the concentration of the PEG-lipid conjugate ranges from about 1 to 3.5% w/w, based on the amounts of total lipid extracts.
   Embodiment 98. The modified PMP formulation of embodiment 86, wherein the sterol is cholesterol having a concentration ranging from about 5 to 8% w/w, based on the amounts of total lipid extracts; and the PEG-lipid conjugate is a C18-PEG2000 PE or its derivative having a concentration ranging from about 1 to 3.5% w/w, based on the amounts of total lipid extracts.
   Embodiment 99. The modified PMP formulation of any one of embodiments 71-98, wherein the modified PMP is a lipid nanoparticle.
   Embodiment 100. The modified PMP formulation of any one of embodiments 71-98, wherein the modified PMP has a size of less than about 200 nm.
   Embodiment 101. The modified PMP formulation of embodiment 100, wherein the modified PMP has a size of about 100 to 160 nm.
   Embodiment 102. The modified PMP formulation of any one of embodiments 71-101, further comprising a phosphate, citrate, sodium bicarbonate, HEPES, TAE, or TRIS buffer at a pH of about 3.0 to about 8.5.
   Embodiment 103. The modified PMP formulation of any one of embodiments 71-102, further comprising one or more cryoprotectants.
   Embodiment 104. The modified PMP formulation of embodiment 103, wherein the one or more cryoprotectants are selected from the group consisting of sucrose, glycerol, mannitol, and a combination thereof.
   Embodiment 105. The modified PMP formulation of any one of embodiments 71-104, wherein the modified PMP formulation is a lyophilized composition.
   Embodiment 106. The modified PMP formulation of any one of embodiments 71-105, wherein the modified PMP formulation is stable at room temperature, and/or at 4° C.
   Embodiment 107. A pharmaceutical composition comprising the modified PMP formulation according to any one of embodiments 71-106 and a pharmaceutically acceptable vehicle, carrier, or excipient.
   Embodiment 108. A method of producing a modified PMP formulation comprising an exogenous peptide, polypeptide, or protein, the method comprising:
providing a solution comprising a modified PMP containing one or more PMPs, one or more sterols, and one or more polyethylene glycol (PEG)-lipid conjugates;
providing a solution comprising the exogenous peptide, polypeptide, or protein; and
loading the modified PMP with the exogenous peptide, polypeptide, or protein, wherein the loading causes the exogenous peptide, polypeptide, or protein to be encapsulated by the modified PMP.
   Embodiment 109. A method for delivering a peptide, polypeptide, or protein to a mammalian cell or a mammal, the method comprising:
contacting the mammalian cell with or administering to the mammal the modified PMP formulation according to any one of embodiments 71-104, under conditions sufficient to allow uptake of the modified PMP formulation by the mammalian cell or by the mammal.
   Embodiment 110. The method of embodiment 109, wherein the method is for delivering a peptide, polypeptide, or protein to a mammalian cell, and the cell is a cell in a subject.
   Embodiment 111. The method of embodiment 109, wherein the exogenous peptide, polypeptide, or protein is released from the modified PMP formulation in the mammalian cell with which the modified PMP formulation is contacted.
   Embodiment 112. The method of embodiment 111, wherein the exogenous peptide, polypeptide, or protein exerts activity in the cytoplasm or nucleus of the mammalian cell.
   Embodiment 113. The method of any one of embodiments 108-112, wherein the mammal is a human.
   Embodiment 114. The method of any one of embodiments 108-112, wherein the uptake by the mammalian cell or by the mammal of the exogenous peptide, polypeptide, or protein encapsulated by the modified PMP formulation is increased relative to the uptake of the exogenous peptide, polypeptide, or protein not encapsulated by a modified PMP formulation.
   Embodiment 115. The method of embodiment 109, wherein the method is for delivering a peptide, polypeptide, or protein to a mammal, and the administration is via oral, intranasal, or intrarectal route.
   Embodiment 116. A method for treating or preventing a disease or disorder in a subject for which a therapeutic agent is indicated, the method comprising administering to the subject in need thereof an effective amount of the modified PMP formulation according to any one of embodiments 71-106, wherein the therapeutic agent is the exogenous peptide, polypeptide, or protein encapsulated by the modified PMP in the modified PMP formulation.
   Embodiment 117. The method of embodiment 116, wherein the administration is via oral, intranasal, or intrarectal route.
   Embodiment 118. The method of embodiment 114, wherein the disease is diabetes, and the exogenous peptide, polypeptide, or protein is insulin.

### EXAMPLES

The following are examples of the various methods of the invention. It is understood that various other embodiments may be practiced, given the general description provided above.

### Example 1: Preparation of PMPs and Complex Lipid Particles

### General Procedure to Prepare PMPs

The preparation of PMPs and formulation of PMPs may be accomplished utilizing similar methods to those disclosed in International Patent Application Publication No. WO 2023/069498, which is incorporated herein by reference in its entirety.

### General Procedure to Prepare Lipid Extract Component of CLPs

The general preparation of complex lipid particles (CLPs) begins with isolation of lipid extract from a natural plant source. Briefly, the method of extraction for plant lipids in CLPs is as follows:
1) Plant matter (e.g. juice, pulp, or a blended part of the plant) is collected from the natural plant source.
2) The plant matter is filtered and concentrated.
3) The plant concentrate is diafiltered with a citrate-sodium chloride buffer to generate Diafiltered Intermediate (DFI), the starting material for extraction.
4) Lipids are extracted by adding an extracting solvent (e.g. dichloromethane (DCM) and methanol (MeOH)). Alternative extracting solvents in this process can include chloroform methanol and ethyl acetate:ethanol.
5) Water is added to induce phase separation.
6) The resulting organic phase is collected and dried using a rotary evaporator (rotovap).
7) The dried lipids are resuspended in an aqueous solution (e.g. 90% DCM and 10% MeOH solution) and are transferred to vials to be dried (e.g. via Genevac or similar drying equipment).
8) The resulting lipid extract is stored dry at −20° C.

### Exemplary Modified Extraction Methods to Prepare CLPs

Modifications to the above extraction of lipids from natural sources are described in detail below. Two alternative extraction methods for the preparation of complex lipid particles were used: 1) an ethanol extraction method, or 2) a modified Matyash method using methanol. In this example, for ethanol extraction, a 3:2 Ethyl Acetate:Ethanol solvent was created. 25 mL of PBS was added to 2.5 g of the powdered natural source then vortexed for a minute at 1500 RPM. 41.5 mL of the ethanol solvent was added to the aqueous sample of a natural source. This was vortexed on a vertical shaker for a minute at 1500 RPM. A further 50 mL of ethyl acetate was added and the solution was vortexed for a minute at 1500 RPM. 50 mL of liquid chromatography-mass spectrometry grade water was added, and the solution was vortexed for another minute at 1500 RPM, then centrifuged for 5 min at 4° C., 1500×g. The top, organic layer was transferred to a new flask, then evaporated using a rotovap with a water bath set to 40° C., making sure to remove any residual solvent. The remaining extracted lipid was weighed, sparged with N2 and then capped and stored at −20° C.

In an alternative example, for the modified methanol extraction, the aqueous sample was prepared as described above. 93.75 mL of a 1:2 MeOH:methyl tert-butyl ether (MTBE) solution was added to the sample, vortexed for a minute at 1500 RPM, then sonicated for 5 minutes at 100% power and 37 Hz. 31.25 mL MTBE was further added, and the solution was vortexed for another minute at 1500 RPM before being centrifuged at 1500×g for 5 min. The top, organic layer was transferred to a flask, and 60 mL ethyl acetate was added. The new solution was vortexed for a minute at 1500 RPM, shaken for 30 seconds, then centrifuged for 5 min at 4° C., 1500×g. The top, organic layer was transferred again. Then, as described for the ethanol extraction, the solvent was evaporated using the rotovap, weighed, and stored.

Dried extracts were reconstituted in absolute ethanol to a given concentration of 10 mg/mL, vortexed in 30 second intervals at 2000 RPM and sonicated in 30 second intervals at 80 Hz. Once the lipids were reconstituted, the sample was filtered into a pre-weighed vial, then dried using a GeneVac before being stored under nitrogen at −20° C.

In addition to the described lipid extraction methods using dichloromethane:methanol, chloroform:methanol, methanol:MTBE, and ethyl acetate:ethanol above, alternative organic solvents that can be used include but are not limited to dimethylformamide:methanol, acetonitrile, acetone, ethanol, methanol, dimethylformamide, tetrahydrofuran, 1-butanol, dimethyl sulfoxide, acetonitrile:ethanol, acetonitrile:methanol, acetone:methanol, methyl tert-butyl ether:propanol, tetrahydrofuran:methanol, or dimethyl sulfoxide:methanol.

### Example 2: Formulation and Characterization of Polypeptide-Loaded Complex Lipid Formulations

In this example, methods were developed to create and load stable complex lipid particles containing complex lipids derived from various plant sources with peptides, polypeptides, and small proteins.

In sum, the methods involved: 1) mixing natural lipids obtained as described in Example 1 with components (e.g., sterol such as cholesterol and poly-ethylene glycol (PEG) molecules) that facilitate increased stability, for preservation of the particle size and amount of encapsulated cargo over time; 2) forming particles containing the above components (either with or without lyophilization); 3) loading the particles either directly or passively with a peptidic cargo; and optionally 4) purifying the resulting peptide-loaded particles for further applications/processing.

Various steps of the processing, formulation, characterization and testing are laid out here and then described in detail below. Steps for programming of a particle may include formulation through microfluids (e.g. using NanoAssemblr) or thin film as described in sections below. Additionally, steps may include direct loading or passive loading of a cargo. Purification steps and post-processing steps may include options such as dialysis, filtration, centrifugation, lyophilization, and/or encapsulation. Testing of formulations may include measures of stability and performance both in vitro and in vivo (e.g., mice, etc). At any of these given stages (e.g., programming, post-processing, testing), characterization of a given formulation (such as size, API loading, lipidomics, etc) may influence further formulations and optional steps to produce an exemplary complex lipid formulation.

### General Procedures to Prepare and Load Empty Complex Lipid Particles

In general, the formulation of a complex lipid particle involves production of empty particles followed by the loading of empty particles or the loading of particles via microfluidic mixing to directly produce loaded particles.

The production of empty particles occurs via either thin film or microfluidic mixing. Briefly, the production of empty particles via thin film hydration involves the following steps: 1) lipid mixing, 2) thin film formation, 3) hydration, 4) homogenization, and 5) empty particle formation. Briefly, the production of empty particles via microfluidics mixing involves the following steps: 1) lipid mixing, 2) microfluidic homogenization, and 3) empty particle formation.

With either thin film or microfluidic mixing, after empty particle formulation, the particles can be optionally lyophilized. If lyophilization does not occur, the empty particle is passively loaded via the following steps: 1) the empty particle mixes with the active pharmaceutical ingredient (API) solution, 2) the subsequent formulation is homogenized, and optionally 3) the formulation is purified. If lyophilization does occur, the empty particle is passively loaded via the following steps: 1) the lyophilized particle is rehydrated in API solution then 2) is homogenized, and then optionally 3) the formulation undergoes purification. Alternatively, the production of loaded particles can occur directly via microfluid mixing. Briefly, the direct loading of particles via microfluidic mixing involves the following steps: 1) lipid mixing with API, 2) microfluidic homogenization, 3) loaded particle formation, and optionally 4) purification of the resulting formulation. After the loading of produced particles through any of the given means, testing of the particle may occur. Testing can include in vitro characterization, in vivo functional tests, lyophilization and capsule packing, or lyophilization and rehydration.

Exemplary CLPs Prepared by Adding Sterol and PEG to Lipids Extracted from Plant Sources

This example described formulating stable particles using lipids derived from plant sources (such as PMP or CLPs). In this example, to the lipids extracted from a plant source was added a sterol (e.g. cholesterol) and PEGylated lipids. Cholesterol works as a molecule to facilitate packing of residual lipids and stabilizing the formulation, but it can increase the rigidity of the lipid particles which may not be desirable especially in cases where the lipid particles were designed to transverse a cellular monolayer, such as the gastrointestinal epithelial layer. PEGylated lipids work as a molecule to prevent fusion of formulated lipid particles. In this example, the amount of added cholesterol and PEGylated lipids were minimized for the following reasons. Natural plant derived sterols are present in many natural lipid compositions. Thus, adding a large amount of exogenous cholesterol may yield rigid lipid particles. Moreover, adding a large amount of PEGylated lipids may increase the propensity of the formulation to be identified by immune cells especially in the cases of repeated administration, rendering the resulting lipid particles less effective.

In this example, the following assumptions were made to calculate the amount of cholesterol and PEG: i) the average molecular weight of the source lipids (natural lipid extract) is 720 Da, ii) 50% the total lipid extract is structural lipids. Thus, for instance, for 1 mg of lipid extract, there was 0.5 mg or 695 nanomoles (nmol) structural lipids. The added cholesterol and PEGylated were calculated relatively to the amount of structural lipids per total mass of the lipid extract.

DSPE-PEG2000 (18:0 PEG2000 PE, CAS #474922-77-5) with a molecular weight 2800 Da was used as PEGylated lipid. Four different concentrations were added to the lipid mix; 0% w/w, 0.5% w/w (5 μg per mg of total lipid extract), 1.25% w/w (12.5 μg per mg total lipid extract), and 2.5% w/w (25 μg per mg of total lipid extract). For 1 mg of total lipid extract or 695 nmol of structural lipids, this translates to 0% mol, 0.26% mol, 0.64% mol, and 1.28% mol PEGylated lipid.

A plant-derived cholesterol (CAS #57-88-5) with a molecular weight 386.5 Da was used as cholesterol. Three different concentrations were added to the lipid mix; 0% w/w, 3.4% w/w (34 μg per mg total lipid extract), and 6.8% w/w (68 μg per mg total lipid extract). For 1 mg of total lipid extract or 695 nmol of structural lipids, this translates to 0% mol, 12.5% mol, and 25% mol of cholesterol.

### Exemplary Production of Empty Complex Lipid Particles

As briefly described above, two main formulation processes for preparing complex lipid particles have been used: 1) Thin Film Rehydration and 2) Microfluidics Enabled.

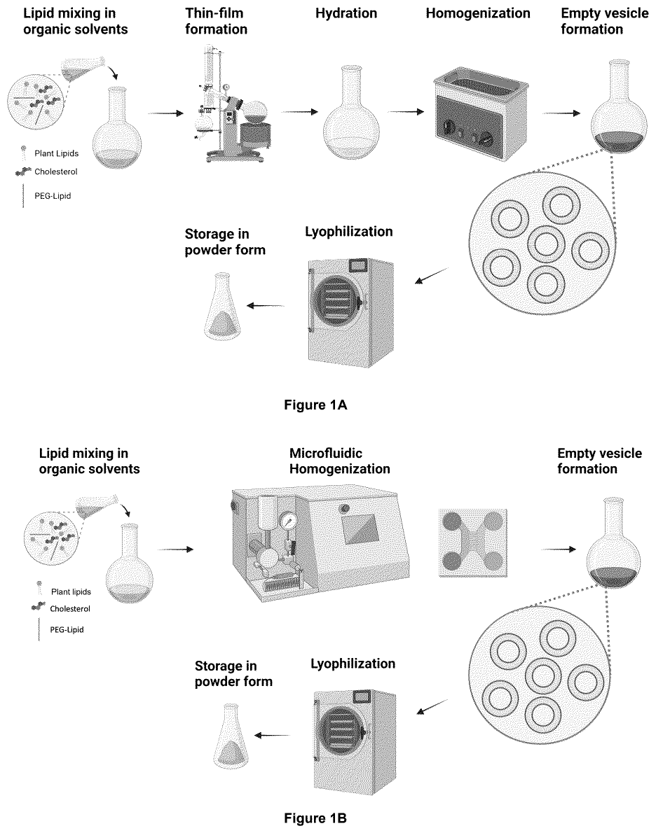

In this example, the formulation process for Thin Film Rehydration involved the following steps, shown in FIG. 1A.
1) The natural source lipids (natural lipids extracted from plants, such as lemon) and exogenous lipids (i.e., cholesterol and PEGylated lipids, as discussed above in various concentrations) were solubilized in an organic solvent (such as chloroform or ethanol). The natural lipids were solubilized at given concentration (eg 5 mg/ml concentration).
2) A thin film of the mixed lipids was formed in a flask by evaporating the organic solvent in a rotary evaporator at 42° C. and further dried using a nitrogen stream.
3) The thin film was hydrated in an aqueous buffer containing a cryoprotectant at 5 mg/ml lipids in buffer at 40° C. for 30 minutes and then vortexed. This buffer was selected to facilitate the downstream solubilization of the protein cargo (e.g., based on the isoelectric point of the protein cargo). For the case of insulin, the buffer was citrate at pH about 3 or sodium bicarbonate at pH about 8.2. For the case of GLP1 receptor agonists (e.g., semaglutide and exenatide), the buffer was 0.1M sodium bicarbonate at pH about 8.2. The cryoprotectant was 2-5% sucrose or 2-5% mannitol. Alternatively, the cryoprotectant could range from between 0% to 5%. The cryoprotectant could be at about 0.5-2%.
4) The lipid film was homogenized in the aqueous buffer in a sonicator for the formation of small unilamellar vesicles (SUVs) containing the natural lipids and exogenous lipids. The sonication process involved two steps: a 20-minute sonication step in 42° C., brief mixing with vortexing, and a second 20-minute sonication step in about 42° C. If aggregates were observed, the particle solution was filtered through a cotton filter. Particle morphological characteristics were analyzed with dynamic light scattering methods.
5) The lipid solution was frozen in liquid nitrogen for 10 minutes. The lipid solution in the aqueous buffer and cryoprotectant was lyophilized (freeze dried) at room temperature overnight or for 12-48 hours depending on the volume of the particle solution. The resulting complex lipid formulations containing the natural source lipids and exogenous lipids were produced in a dry powder form that was stored in −20° C. until use. The particle could be lyophilized in an alternative method known as shelf lyophilization, in which the particles are controlled through the freezing process using vacuum rate. Alternatively, the particles were not lyophilized.

The formulation process for Microfluidics Enabled processing involved the following steps, shown in FIG. 1B.
1) The natural lipids and exogenous lipids (i.e., cholesterol and PEGylated lipids, as discussed above in various concentrations) were solubilized in organic solvents at a given concentration (e.g. 5 mg/ml concentration).
2) The lipid mix in an organic solvent was co-injected in a microfluidic device (NanoAssemblr Ignite) with an aqueous buffer (e.g. 0.1M sodium bicarbonate at a pH of 8.2 supplemented with 5% mannitol as a cryoprotectant).
3) The organic solvent was dialyzed out overnight against the aqueous buffer (e.g. 0.1M sodium bicarbonate, pH 8.2, with 5% mannitol as a cryoprotectant).
4) Particle morphological characteristics were analyzed with dynamic light scattering methods.
5) The lipid solution was frozen in liquid nitrogen for 10 minutes. The lipid solution was lyophilized (freeze dried) at room temperature overnight. The resulting complex lipid particles containing the natural source lipids and exogenous lipids were produced in a dry powder form that was stored in −20° C. until use. The particle could be lyophilized in an alternative method known as shelf lyophilization, in which the particles are controlled through the freezing process using vacuum rate. Alternatively, the particles were not lyophilized.

Other known methods, such as microfluidic T-junction production, may be used instead.

Loading a Polypeptide into Empty Complex Lipid Particles

The complex lipid particles containing the natural source lipids and exogenous lipids, produced in a dry powder form and stored in −20° C. and prepared according to the above formulation processes, were retrieved to formulate with an exemplary active biomolecule (e.g., a polypeptide such as insulin).

In this example, the lyophilized particles were re-hydrated with an aqueous buffer containing the active biomolecule (e.g., a polypeptide such as insulin) at 5 mg/ml concentration for peptides smaller than or equal to 5 kD for a 1:1 w/w lipid-to-active-biomolecule ratio, or at 1 mg/mL for proteins larger than 5 kD for a 1:5 w/w lipid:peptide mass ratio. Rehydration was followed with brief homogenization with sonication for 20 minutes at 40° C. This rehydration buffer was similar to the one used to hydrate the thin film (step 3 above) and was selected based on its properties to solubilize the active biomolecule. Again, for the case of insulin, the buffer was citrate at pH about 3 or sodium bicarbonate at pH about 8.2; for the case of GLP1 receptor agonists (e.g., semaglutide or exenatide), the buffer was 0.1M sodium bicarbonate at pH about 8.2. Particle morphological characteristics were analyzed with dynamic light scattering methods. Alternatively, if the particle was not lyophilized, the empty particles were mixed with an aqueous buffer containing the active biomolecule (e.g. a polypeptide such as insulin) in a similar manner to the methods described above.

Next, the mixture of re-hydrated particles containing the active biomolecule was purified and separated from the free active biomolecule not loaded in the particles by a dialysis step overnight. The dialysis was performed in a 100 kD dialysis membrane in the buffer used for the formulation process as discussed above, at a buffer volume at least 2000× the volume of the solution to be purified (e.g., 0.1M sodium bicarbonate, pH 8.2, buffer). The dialysate was concentrated in centrifugal unit to the preferred concentration. Alternatively, the purification and separation step could be performed with tangential flow filtration (TFF). The described purification steps are optional and were not performed for all formulations. Particle morphological characteristics were analyzed with dynamic light scattering methods. Particle loading efficiency was quantified with a bicinchoninic acid assay (BCA).

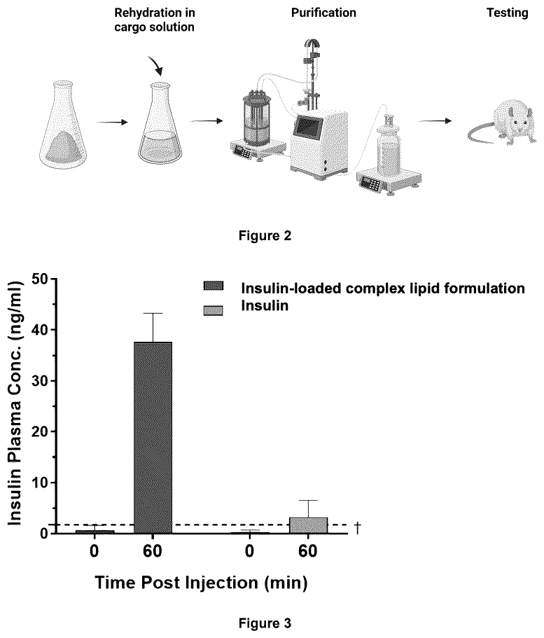

The above process for loading a bioactive molecule into empty complex lipid particles containing the natural source lipids and exogenous lipids is also shown in FIG. 2.

An alternative method for loading a bioactive molecule into complex lipid particles containing the natural source lipids and exogenous lipids involves solubilizing the lipid mix in organic solvents together with an aqueous buffer containing the active biomolecule, before homogenizing the mixed solutions via microfluidics to produce a loaded particle formulation directly. In another method, the bioactive molecule may also be solubilized in the organic phase.

### Characterization of Polypeptide-Loaded Complex Lipid Formulation

The efficacy of the complex lipid formulations, prepared according to above formulation and loading processes, was assessed by quantitative methods characterizing the size of the particles and the distribution of the size among the particle population. The particle diameter was measured with dynamic light scattering (DLS) measurements. The results are shown in Table 5. Table 5 lists the particle diameter (nm) as measured with DLS for the complex lipid formulations, described above, by varying the amounts of added cholesterol (left column) and PEGylated lipids (top row) in the complex lipid formulations.

TABLE 5
Particle sizes of the insulin-loaded complex lipid formulations.

PEG (% w/w)

| Chol(% w/w) | 0 | 0.5 | 1.25 | 2.5 |
|---|---|---|---|---|
| 0 | 120 | 139 | 129 | 113 |
| 3.4 | 434 | 137 | 128 | 144 |
| 6.8 | 445 | 149 | 138 | 102 |

As shown in Table 5, adding cholesterol alone resulted in a much larger particle size, and adding PEGylated lipids alone also slightly increased the particle size when the amount of PEGylated lipids was 0.5% w/w. The insulin-loaded complex lipid formulations had a smaller particle size when the complex lipid formulations contained 2.5% w/w PEGylated lipids and 6.8% w/w cholesterol, based on the amounts of total lemon lipid extracts.

The complex lipid formulations containing 2.5% w/w PEGylated lipids and 6.8% w/w cholesterol were able to provide a particle size ranging from 100-160 nm, when the above formulation and loading processes were repeated for multiple times (e.g., hundreds of times).

The insulin encapsulation efficiency of the complex lipid formulations (the amount of insulin that was trapped to the vesicles) was also characterized, and the results ranged consistently from 25-35%. Similar characterizations were measured for other formulations of complex lipid particles.

### Stability of Polypeptide-Loaded Complex Lipid Formulations

As shown in Table 5, as a control, the insulin-loaded complex lipid formulation without including exogenous lipids (i.e., 0% w/w PEGylated lipids and 0% w/w cholesterol) initially had a small particle size of about 120 nm. However, this formulation was unstable over time, and the particle size increased to 400-500 nm after storing at 4° C. for a day. Moreover, lyophilization is typically used for a longer time storage of the formulation (e.g., more than two weeks), but the protein-loaded complex lipid formulation without including exogenous lipids (i.e., 0% w/w PEGylated lipids and 0% w/w cholesterol) was unstable after a second lyophilization and the particle size increased to 400-500 nm after the second lyophilization. In addition, the protein-loaded complex lipid formulation without including exogenous lipids (i.e., 0% w/w PEGylated lipids and 0% w/w cholesterol) could not be reformulated upon rehydration of the powder with a buffer solution.

On the other hand, the complex lipid formulations containing the PEGylated lipids and cholesterol were stable at 4° C. at least for two weeks. For a longer storage time, lyophilization was used. The complex lipid formulations containing the PEGylated lipids and cholesterol were stable after lyophilization. The complex lipid formulations containing the PEGylated lipids and cholesterol were also capable of being reformulated upon rehydration of the powder with buffer solution. After the reformulation, the particle size of the complex lipid formulations was able to achieve about 90% of the original particle size prior to lyophilization, and the insulin encapsulation efficiency of the complex lipid formulations was able to achieve about 30% of the initial encapsulation efficiency prior to lyophilization. Similar measures of stability were used for other formulations of complex lipid particles.

### Lipidomics of Plant Lipid Extracts and Empty Complex Lipid Particles

In this example, lipids were extracted from five plant sources (e.g. lemon, dragon fruit, spinach, kale, strawberry) as described in Example 1. These extracts were either formulated into empty complex lipid particles as described in Example 2 above or were maintained as a lipid extract composition. Both extracts and empty particles underwent lipidomics analysis.

Briefly, extracts or particles were solubilized in compatible solvents and analyzed by MS/MS. Prevalence of determined lipid classes and subclasses identified in extracts vs particles was analyzed. The 46 different lipid classes/subclasses annotated via lipidomics were the following: acyl diacylglyceryl glucuronides, acylhexosylceramides, acylsterylglycosides, bile acids, acyl carnitines, Cholesteryl esters, ceramides, Cardiolipins, Coenzyme Qs, Diacylglycerols, Digalactosyldiacylglycerols, Diacylglyceryl glucuronides, Dilysocardiolipins, fatty acids, Fatty acid ester of hydroxyl fatty acids, Hemibismonoacylglycerophosphates, hexosylceramides, Lysophosphatidic acids, Lysophosphatidylcholines, Lysophosphatidylethanolamines, N-acyl-lysophosphatidylethanolamines, Lysophosphatidylglycerols, Lysophosphatidylinositols, lysophosphatidylserines, Monogalactosyldiacylglycerols, Lysocardiolipins, N-acyl ethanolamines, N-acyl glycines, N-acyl glycyl serines, phosphatidic acids, phosphatidylcholines, phosphatidylethanolamines, phosphatidylethanols, phosphatidylglycerols, phosphatidylinositols, ceramide phosphoinositols, phosphatidylmethanols, phosphatidylserines, steryl esters, stigmasterols, sulfatides, sulfonolipids, sphingomyelins, Sulfoquinovosyl diacylglycerols, sterols, and triacylglycerols.

The number of lipids annotated in lemon extract alone was over 800, and these lipids were categorized into at least 20 of the above listed classes or subclasses. This highlights the complexity of the natural source lipid extract compositions, which was evident among all five of the tested plant sources. The prevalence of certain lipid classes or subclasses changed through the formulation process, as was evident when comparing the analyzed lipids present in the extract or the empty complex lipid particle. Even with changes in lipid class or subclass prevalence during the formulation process, the overall diversity of lipid classes/subclasses was not lost.

Across the five tested empty particle formulations, an average of 24 of the listed lipid classes was represented among the positive charged particle analysis, and an average of 31 of the annotated lipid classes was represented among the negative charged particle analysis. Between different sources, a minimum of 5 annotated lipid classes/subclasses overlapped within the particle analysis, with some sources having a minimum of 20 overlapping lipid classes/subclasses within the particle.

Of note, the nonlipid components found within the lipid extract were not inspected within the lipidomics analysis. That said, characterization of lemon lipid extract found that across multiple extractions, the total protein content was less than 10% w/w as measured by BCA. In some cases, the total protein content of the lipid extract was less than 5% w/w of the extract composition. Additional characterization of the lemon lipid extract found that the total dsDNA was less than 1% w/w of the extract composition as measured by Picogreen assay. In some cases the total dsDNA was less than 0.05% w/w of the extract composition.

### Example 3: Production of Oral Dosage Form of a Complex Lipid Formulation

The following example describes how a capsule or tablet dosage form is produced from a complex lipid formulation.

First, for a capsule dosage form, a liquid complex lipid formulation is obtained according to the procedures described in Example 2. Additional excipients can be added, including, but not limited to, cryoprotectants, lyoprotectants, stabilizers, bulking agents, flowing agents, and/or anti-caking agents. The liquid formulation is then dried into a solid formulation using established pharmaceutical manufacturing procedures, such as freeze drying or spray drying. If the dried material is a free-flowing powder, it is filled in a capsule using standard manufacturing procedures. If the dried material is not free flowing, as in the case of a lyophilized cake, procedures such as milling are used to produce a powder from the cake. The powder is then filled in a capsule using standard manufacturing procedures. The capsule shell may be uncoated or coated. The capsules may be bulk packaged or individually packaged.

First, for a tablet dosage form, a liquid complex lipid formulation is obtained according to the procedures described in Example 2. Additional excipients can be added, including but not limited to cryoprotectants, lyoprotectants, stabilizers, bulking agents, flowing agents, binding agents, compressibility agents, and/or anti-caking agents. The liquid formulation is then dried into a solid formulation using established pharmaceutical manufacturing procedures, such as freeze drying or spray drying. If the dried material is a free-flowing powder, it is compressed into a tablet using standard manufacturing procedures. If the dried material is not free flowing, as in the case of a lyophilized cake, procedures such as milling are used to produce a powder from the cake. The powder is then compressed into a tablet using standard manufacturing procedures. The tablets may be uncoated or coated. The tablets may be bulk packaged or individually packaged.

### Example 4: In Vivo Intranasal and Enteral Delivery of the Protein-Loaded Complex Lipid Formulations

The insulin-loaded complex lipid formulation was obtained according to Example 2, in which the complex lipid formulation contained 2.5% w/w PEGylated lipids and 6.8% w/w cholesterol, based on the amounts of total lemon lipid extracts.

The animals used were C57 female 8-week-old mice (Jax #0664).

### Intracolonic Experiment

The mice were administered with the insulin-loaded complex lipid formulations via an intrarectal dose at 50 μL (50 U/kg), according to the treatment schedule in Table 6. At various time points, as indicated in Table 6, blood was collected and plasma samples were obtained, and the insulin levels were quantified by ELISA assay.

TABLE 6
Treatment schedule for intracolonic administration

| Group | Group | Days of | Route & | |
|---|---|---|---|---|
| # | Size | Dosing | Frequency | Evaluation & Endpoint |
| 1 | 5 | 1 day | Once | Procedure: |
| 2 | 5 | 1 day | Once | Day 0: |
| 3 | 5 | 1 day | Once | Overnight fasting |
| 4 | 5 | 1 day | Once | Day 1 |
| | | | | Pre Dose T = 0 Blood collection |
| | | | | (50 μL) → plasma |
| | | | | Intrarectal TA delivery |
| | | | | Blood collection (50 μl): 0.5 h, |
| | | | | 1 h → plasma |
| | | | | Blood collection cardiac |
| | | | | puncture: 2 hr → plasma |

The results are shown in FIG. 3. FIG. 3 shows the insulin concentration in the plasma of the mice at 1 hour after intrarectal administration of the insulin-loaded complex lipid formulations to the mice, illustrating the systemic absorption of the insulin-loaded complex lipid formulations. As compared to the benchmark for fusogenic liposomes containing insulin administered directly into colon (10 U/kg, 12 minutes post injection), intrarectal administration of the insulin-loaded complex lipid formulations resulted in a significantly higher plasma level of insulin, compared to the benchmark.

### Intranasal Experiment

The mice were administered with the insulin-loaded complex lipid formulations via an intranasal dose at 40 μL (20 μL pernostril, 50 U/kg), according to the treatment schedule in Table 7. On various time points as indicated in Table 7, brains were weighed, and protein was extracted using acidified ethanol. The insulin levels were quantified by ELISA assay.

TABLE 7
Treatment schedule for intranasal administration

| Group | Group | Days of | Route & | |
|---|---|---|---|---|
| # | Size | Dosing | Frequency | Evaluation & Endpoint |
| 1 | 5 | 1 Day | Once | Procedure: |
| 2 | 5 | 1 day | Once | Day 0: |
| 3 | 5 | 1 day | Once | Overnight fasting |
| 4 | 5 | 1 day | Once | Day 1: |
| | | | | Pre-Dose T = 0 Blood collection |
| | | | | (50 μL) → plasma |
| | | | | Intra Rectal TA delivery |
| | | | | Blood collection (50 μl): 0.5 h, |
| | | | | 1 h → plasma |
| | | | | Blood collection cardiac |
| | | | | puncture: 2 hr → plasma |
| | | | | Collect brain → on dry ice |

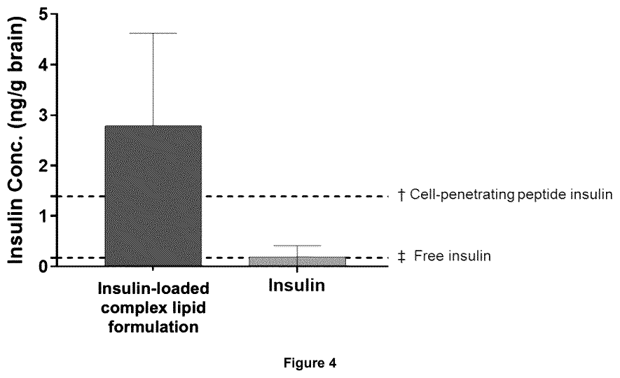

The results are shown in FIG. 4. FIG. 4 shows the insulin concentration in the brain of the mice at 2 hours after intranasal administration of the insulin-loaded complex lipid particles to the mice, demonstrating an effective brain delivery of insulin via intranasal administration of the insulin-loaded complex lipid particles. As compared to the benchmark for intranasal administration of free insulin as well as for intranasal administration of insulin with cell-penetrating peptides, intranasal administration of the insulin-loaded complex lipid particles resulted in a significantly higher brain level of insulin, compared to both benchmarks.

### Example 5: In Vivo Enteral Delivery of Protein-Loaded Complex Lipid Formulations

The insulin-loaded complex lipid formulations were obtained according to Example 2, in which the complex lipid particles were formulated as described in Table 8.

TABLE 8
Insulin-loaded complex lipid formulations.

| Formulation | PEG | | Sterol | | Lipid Extract Component | |
|---|---|---|---|---|---|---|
| Number | Name | Mass % | Name | Mass % | Name | Mass % |
| 1 | PEG | 2 | Cholesterol | 28 | Lemon | 70 |
| 2 | PEG | 2 | Cholesterol | 16 | Lemon | 82 |
| 3 | PEG | 2 | Cholesterol | 39 | Lemon | 58 |
| 4 | PEG | 4 | Sitosterol | 39 | Lemon | 57 |
| 5 | PEG | 3 | Sitosterol | 57 | Lemon | 41 |
| 6 | PEG | 5 | Cholesterol | 27 | Lemon | 68 |
| 7 | PEG | 2 | Cholesterol | 6 | Lemon | 91 |
| 8 | PEG | 2 | Cholesterol | 23 | Soy | 75 |
| 9 | PEG | 1 | Cholesterol | 28 | Lemon | 71 |
| 10 | PEG | 3 | Cholesterol | 6 | Kale | 91 |
| 11 | PEG | 3 | Cholesterol | 6 | Dragon Fruit | 91 |
| 12 | PEG | 3 | Cholesterol | 6 | Spinach | 91 |
| 13 | PEG | 3 | Cholesterol | 6 | Strawberry | 91 |
| 14 | PEG | 15 | Sitosterol | 48 | Lemon | 37 |

### Intracolonic Experiment

The animals used were C57BL6 female 8-week-old mice (Jax #0664).

C57BL6 female 8-week-old mice (Jax #0664) were used (n=4-5 mice/formulation). Mice were weighed, and a pre-bleed sample was taken (through tail vein, retro-orbital or submandibular means). The mice were fasted overnight before being dosed via intracolonic administration with 50 uL of a given insulin-loaded complex lipid formulation (Table 8). Non-terminal bleeds at 30 minutes and 60 minutes post-dose were taken, alternating routes of collection. A terminal bleed at 120 minutes post-dose via cardiac puncture was also taken. Whole blood samples from the pre-bleed, 30 minute, 60 minute, and 120 minute bleeds were processed for plasma then stored at −80° C. Insulin levels were quantified by MSD assay. Particle characterization and insulin measures are reported in Table 9.

TABLE 9
Particle characterization and insulin levels for insulin-loaded complex lipid formulations.

| | | | | DLS - | | | | | | | |
|---|---|---|---|---|---|---|---|---|---|---|---|
| | | Dose | DLS - | Concen- | | BCA | | | | | |
| Formulation | | Volume | Size | tration | DLS - | (Insulin | | | | | |
| Number | ROA | (mL) | (nm) | (p/mL) | PDI | mg/mL) | Mouse 1 | Mouse 2 | Mouse 3 | Mouse 4 | Mouse 5 |
| Control | IC | 0.05 | — | — | — | Avg = 1.16 | Avg = 290.1 | | | | |
| 1 | IC | 0.05 | 89.92 | 2.71E+13 | 0.26 | 0.99 | 25347.5 | 5436.3 | 12218.9 | 8569.1 | 12294.8 |
| 2 | IC | 0.05 | 97.22 | 2.51E+12 | 0.26 | 0.83 | 2607.1 | 1950.0 | 741.0 | 1361.6 | 335455.0 |
| 3 | IC | 0.05 | 85%, 115 | 2.10E+11 | NA | 1.20 | 222.5 | 753.2 | 1513.4 | 1095.5 | 1346.1 |
| 4 | IC | 0.05 | 134.4 | 3.19E+11 | NA | 0.45 | 341811.0 | 2501.8 | 815.4 | 135.6 | 387.8 |
| 5 | IC | 0.05 | 184.4 | 4.65E+10 | NA | 0.30 | 1529.9 | 337.2 | 384.8 | 208.1 | 128.3 |
| 6 | IC | 0.05 | 116.7 | 1.47E+12 | NA | 1.00 | 4167.5 | 123279.0 | 3713.5 | 2395.6 | 3262.4 |
| 7 | IC | 0.05 | 149.3 | 3.57E+11 | NA | 0.88 | 11050.3 | 22681.1 | 6050.5 | 66764.5 | 69334.6 |
| 8 | IC | 0.05 | 130.8; 26.89 | 1.53E+12 | 0.3118 | 0.73 | 17.9 | 99.9 | 62.3 | 30.8 | 161.7 |
| 9 | IC | 0.05 | 107 | 7.57E+11 | 0.3146 | 0.32 | 169.4 | 383.5 | 423.4 | 310.1 | 830.6 |
| 10 | IC | 0.05 | 120.3; 486.4 | 1.08E+12 | 0.226 | 0.45 | 3200.6 | 412.2 | 421.4 | 1055.2 | 523.6 |
| 11 | IC | 0.05 | 85.5; 15.86; 450.7 | 4.67E+14 | 0.3675 | 0.48 | 8599.9 | 3097.3 | 1388.9 | 1081.0 | 2220.4 |
| 12 | IC | 0.05 | 142.9; 35.16 | 8.20E+13 | 0.2981 | 0.26 | 784977.2 | 279.1 | 214.5 | 252.2 | 378.5 |
| 13 | IC | 0.05 | 241.2; 439.9 | 1.39E+10 | 0.5055 | 0.66 | 331478.8 | 570.1 | 1566.3 | 943.0 | 523.5 |
| 14 | IC | 0.05 | 161.6 | 5.52E+11 | 0.1668 | 0.66 | 206.3 | 499.7 | 643.4 | 506829.7 | 838.4 |

Exposure to the active biomolecule (e.g. insulin) was measured as area under the curve (AUC) of the four measured timepoints, and dose was measured as BCA*dose volume (0.05 mL). Therefore, the data in Table 9 for each mouse is shown as systemic levels of insulin (or exposure normalized by dose). As shown in Table 9, 22/22 different complex lipid formulations showed positive systemic levels of insulin. Unexpectedly, 20 of 22 (>90%) different complex lipid formulations out-performed free IC insulin.

### Example 6: In Viva Intrajejunal Delivery of the Protein-Loaded Complex Lipid Formulations

The exenatide-loaded complex lipid formulations were obtained according to Example 2, in which the complex lipid particles were formulated as described in Table 10. Mice were treated according to Table 11.

TABLE 10
Exenatide-loaded complex lipid formulations.

| | | | | | Lipid Extract | | | |
|---|---|---|---|---|---|---|---|---|
| Formulation | PEG | | Sterol | | Component | | Cargo | |
| Number | Name | mg | Name | mg | Name | mg | Name | mg |
| 15 | DSPE | 0.0625 | Cholesterol | 0.34 | Strawberry | 5 | Exenatide | 5 |
| 16 | PEG- | 0.0625 | | 0.34 | Broccoli | 5 | | 5 |
| 17 | 2k | 0.0625 | | 0.34 | Spinach | 5 | | 5 |

TABLE 11
Treatment schedule for intrajejunal dosing.

| | Group | Days of | Route & | |
|---|---|---|---|---|
| Group | Size | Dosing | Frequency | Evaluation & Endpoint |
| 15 | 5 | 1 Day | IJ, Once | Procedure: |
| 16 | 5 | 1 day | IJ, Once | Day 0: |
| 17 | 5 | 1 day | IJ, Once | Overnight fasting |
| | | | | Day 1: |
| | | | | Pre-Dose T = 0 Blood collection |
| | | | | (50 μL) → plasma |
| | | | | Intrajejunal delivery |
| | | | | Blood collection (50 μl): 30 min, |
| | | | | 60 min → plasma |
| | | | | Blood collection cardiac |
| | | | | puncture: 120 min → plasma |

### Intrajejunal Experiment

The animals used were C57BL6 female 8-week-old mice (Jax #0664).

C57BL6 female 8-week-old mice (Jax #0664) were used (n=5 mice/formulation). Mice were weighed and a pre-bleed sample was taken (through tail vein, retro-orbital or submandibular means). The mice were fasted overnight before being dosed via intrajejunal administration with 50 uL (50 ug/mouse) of a given exenatide-loaded complex lipid formulation (Table 10). Non-terminal bleeds at 30 minutes and 60 minutes post-dose were taken, alternating routes of collection. A terminal bleed at 120 minutes post-dose via cardiac puncture was also taken. Whole blood samples from the pre-bleed, 30 min, 60 min, and 120 minute bleeds were processed for plasma then stored at −80° C. Exenatide levels were quantified by ELISA assay. Formulations are reported in Table 10, and treatment schedule is shown in Table 11. The results of particle characterization and exenatide measures are shown in Table 12.

TABLE 12
Particle characterization and insulin levels for insulin-loaded complex lipid formulation.

| | | Dose | DLS - | DLS - | | | | | | | |
|---|---|---|---|---|---|---|---|---|---|---|---|
| Formulation | | Volume | Size | Concentration | DLS - | Exenatide | Mouse | Mouse | Mouse | Mouse | Mouse |
| Number | ROA | (mL) | (nm) | (p/mL) | PDI | mg/mL | 1 | 2 | 3 | 4 | 5 |
| 15 | IJ | 0.05 | 151.5 | 3.16E+13 | 0.29 | 2.86 | 3676.8 | 173.4 | 117.2 | 2168.1 | 617.6 |
| 16 | IJ | 0.05 | 148.3 | 1.89E+13 | 0.39 | 0.87 | 413.2 | 546.2 | 4617.0 | 455.0 | 2297.5 |
| 17 | IJ | 0.05 | 174.9 | 3.45E+14 | 0.23 | 1.30 | 766.2 | 86.3 | 29.0 | 17.5 | 28.2 |

Exposure to the active biomolecule (e.g. exenatide) was measured as area under the curve (AUC) of the four measured timepoints, and does was measured as BCA*dose volume (0.05 mL). Therefore, the data in Table 12 for each mouse is shown as systemic levels of insulin (or exposure normalized by dose). As shown in Table 12, results indicate complex lipid formulations incorporating plant lipid extracts from both broccoli and strawberry performed well when delivering exenatide.

### OTHER EMBODIMENTS

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference. Other embodiments are within the claims.

1. A method for delivering a therapeutic peptide or protein to a human subject in need thereof, the method comprising orally or enterally administering to the human subject a pharmaceutical preparation comprising:
(a) a plurality of complex lipid particles characterized by: (i) comprising at least 10 plant lipids extracted from one or more plant sources; (ii) comprising a sterol exogenous to the one or more plant sources, (iii) comprising a polyethylene glycol (PEG)-conjugated lipid; (iii) containing less than 10% w/w of protein matter endogenous to the one or more plant sources; and (iv) containing less than 10 mol % of exogenous ionizable lipids, and
(b) the therapeutic peptide or protein encapsulated in the complex lipid particles.

2. The method of claim 1, wherein the therapeutic peptide or protein is a hormone or glucagon-like peptide 1 (GLP-1) agonist

3-6. (canceled)

7. The method of claim 1, wherein the complex lipid particle contains less than 5% w/w of protein matter endogenous to the one or more plant sources, and/or less than 5 mol % of exogenous ionizable lipids.

8-10. (canceled)

11. A complex lipid formulation, comprising:
a plurality of complex lipid particles, each complex lipid particle of the plurality comprising at least five lipids extracted from one or more plant sources and at least two exogenous lipids; and
one or more exogenous peptides, polypeptides, or proteins, encapsulated in the complex lipid particles, wherein the complex lipid particles are characterized by one or more of the following characteristics:
i) containing less than 50% w/w of protein matter endogenous to the one or more plant sources; and
ii) containing less than 50 mol % of ionizable lipids.

12. (canceled)

13. The complex lipid formulation of claim 11, wherein the exogenous peptide, polypeptide, or protein is an antibody or an antibody fragment; a hormone; or a receptor agonist or a receptor antagonist.

14-18. (canceled)

19. The complex lipid formulation of claim 11, wherein the exogenous peptide, polypeptide, or protein has a size of less than 100 kD, optionally comprising at least 30 amino acid residues.

20-23. (canceled)

24. The complex lipid formulation of claim 11, wherein the complex lipid particle contains at least 10 plant lipids belonging to one or more of the classes selected from the group consisting of glycerolipid, sphingolipid, and sterol.

25. The complex lipid formulation of claim 24, wherein the complex lipid particle contains:
one or more glycerolipids selected from the group consisting of phospholipids (PL), galactolipids (GL), triacylglycerols (TG), and sulfolipids (SL); and/or
one or more sphingolipids selected from the group consisting of glycosyl inositolphosphoceramides (GIPC), glucosylceramides (GCer), ceramides (Cer), and free long-chain bases (LCB); and/or
one or more sterols selected from the group consisting of campesterol, stigmasterol, and sitosterol.

26-27. (canceled)

28. The complex lipid formulation of claim 24, wherein the complex lipid particle contains one or more lipids belonging to one or more of the sub-classes selected from the group consisting of acyl diacylglyceryl glucuronides, acylhexosylceramides, acylsterylglycosides, bile acids, acyl carnitines, cholesteryl esters, ceramides, cardiolipins, coenzyme Qs, diacylglycerols, digalactosyldiacylglycerols, diacylglyceryl glucuronides, dilysocardiolipins, fatty acids, fatty acid esters of hydroxyl fatty acids, hemibismonoacylglycerophosphates, hexosylceramides, lysophosphatidic acids, lysophosphatidylcholines, lysophosphatidylethanolamines, N-acyl-lysophosphatidylethanolamines, lysophosphatidylglycerols, lysophosphatidylinositols, lysophosphatidylserines, monogalactosyldiacylglycerols, lysocardiolipins, N-acyl ethanolamines, N-acyl glycines, N-acyl glycyl serines, phosphatidic acids, phosphatidylcholines, phosphatidylethanolamines, phosphatidylethanols, phosphatidylglycerols, phosphatidylinositols, ceramide phosphoinositols, phosphatidylmethanols, phosphatidylserines, steryl esters, stigmasterols, sulfatides, sulfonolipids, sphingomyelins, sulfoquinovosyl diacylglycerols, sterols, and triacylglycerols.

29. (canceled)

30. The complex lipid formulation of claim 28, wherein the complex lipid particle contains lipids from at least five, at least six, at least seven, at least eight, at least nine, or at least ten different sub-classes.

31. The complex lipid formulation of claim 11, wherein the complex lipid particle contains less than 30% w/w of protein matter endogenous to the one or more plant sources, and/or less than 20 mol % of exogenous ionizable lipids.

32-38. (canceled)

39. The complex lipid formulation of claim 11, wherein the exogenous lipids comprise a sterol and a polyethylene glycol (PEG)-lipid conjugate.

40-42. (canceled)

43. The complex lipid formulation of claim 39, wherein the exogenous lipids further comprise a lipid selected from the group consisting of a fatty acid, a glycerolipid, a glycerophospholipid, a sphingolipid, a second sterol, and an additive synthetic lipid.

44. The complex lipid formulation of claim 39, wherein the complex lipid particle comprises:
about 10-95% w/w of the plant lipids,
about 5-60% w/w of the sterol, and
about 0.5-15% w/w the polyethylene glycol (PEG)-lipid conjugate, based on the amounts of total lipids in the complex lipid formulation.

45. (canceled)

46. The complex lipid formulation of claim 19, wherein the complex lipid particles have an average size of less than about 250 nm, and/or a PDI of about 0.1 to about 0.5.

47-50. (canceled)

51. The complex lipid formulation of claim 11, wherein the complex lipid formulation is a lyophilized composition or a liquid composition.

52-53. (canceled)

54. A pharmaceutical composition comprising the complex lipid formulation according to claim 11, and a pharmaceutically acceptable vehicle, carrier, or excipient.

55. (canceled)

56. A method for delivering a peptide, polypeptide, or protein to a mammalian cell or a mammal, the method comprising:
contacting the mammalian cell with or administering to the mammal a complex lipid formulation, under conditions sufficient to allow uptake of the complex lipid formulation by the mammalian cell or by the mammal,
wherein the complex lipid formulation comprises:
a plurality of complex lipid particles, each complex lipid particle of the plurality comprising at least five lipids extracted from one or more plant sources and at least two exogenous lipids, and
one or more exogenous peptides, polypeptides, or proteins, encapsulated in the complex lipid particles, wherein the complex lipid particles are characterized by one or more of the following characteristics:
i) containing less than 50% w/w of protein matter endogenous to the one or more plant sources; and
ii) containing less than 50 mol % of ionizable lipids.

57-58. (canceled)

59. The method of claim 56, wherein the method is for delivering a peptide, polypeptide, or protein to a mammal, and the administration is via oral, enteral, intranasal, intracolonic, intrarectal, or intrajejunal route.

60. The method of claim 56, wherein the mammalian cell is brain cell.

61. A method for treating or preventing a disease or disorder in a subject for which a therapeutic agent is indicated, the method comprising:
administering to the subject in need thereof an effective amount of a complex lipid formulation comprising:
a plurality of complex lipid particles, each complex lipid particle of the plurality comprising at least five lipids extracted from one or more plant sources and at least two exogenous lipids, and
one or more exogenous peptides, polypeptides, or proteins, encapsulated in the complex lipid particles, wherein the complex lipid particles are characterized by one or more of the following characteristics:
i) containing less than 50% w/w of protein matter endogenous to the one or more plant sources; and
ii) containing less than 50 mol % of ionizable lipids.

62. The method of claim 61, wherein the administration is via oral, enteral, intranasal, intracolonic, intrarectal, or intrajejunal route.

63. The method of claim 61, wherein the disease is diabetes, and the exogenous peptide, polypeptide, or protein is insulin, exenatide, semaglutide, or tirzepatide.

64. A method of producing a complex lipid formulation comprising a plurality of complex lipid particles encapsulating an exogenous peptide, polypeptide, or protein, the method comprising:
extracting at least five lipids from one or more plant sources;
mixing at least two exogenous lipids with the extracted plant lipids to form complex lipid particles; and
loading the complex lipid particles with the exogenous peptide, polypeptide, or protein, wherein the loading causes the exogenous peptide, polypeptide, or protein to be encapsulated by the complex lipid particles, thereby forming the complex lipid formulation;
wherein:
the extracting step further comprises reducing or eliminating protein matter endogenous to the one or more plant sources to less than 50% w/w, and/or
the exogenous lipids do not include an ionizable lipid.

65-70. (canceled)
